(19) United States
(12) Patent Application Publication
(10) Pub. No.: US 2023/0159861 A1
(43) Pub. Date: May 25, 2023

(54) ENZYME COMPOSITIONS AND USES THEREOF

(71) Applicant: Novozymes A/S, Bagsvaerd (DK)

(72) Inventors: Iben Damager, Vaerloese (DK); Lise Munch Mikkelsen, Roedovre (DK); Henrik Lund, Copenhagen (DK)

(73) Assignee: Novozymes A/S, Bagsvaerd (DK)

(21) Appl. No.: 17/759,272

(22) PCT Filed: Jan. 19, 2021
(86) PCT No.: PCT/EP2021/050978

§ 371 Date: Jul. 21, 2022

(30) Foreign Application Priority Data
Jan. 23, 2020 (EP) 20153364.3

(51) Int. Cl. C11D 3/386; C12N 9/24; C12N 9/22

(52) U.S. Cl. CPC C11D 3/38645; C12N 9/2405; C12N 9/22; C12Y 302/01006; C12Y 301/21001; C12Y 302/01001; C12Y 302/01004; C12Y 302/01078; C12Y 301/01003; C12Y 304/21014; C12Y 302/01015

(57) ABSTRACT

The present invention relates to compositions such as cleaning compositions comprising a mix of enzymes. The invention further relates to use of compositions comprising such enzymes in cleaning processes.

### REFERENCE TO A SEQUENCE LISTING

This application contains a Sequence Listing in computer readable form, which is incorporated herein by reference.

### BACKGROUND OF THE INVENTION

The present invention relates to enzyme compositions such as cleaning compositions comprising a mix of enzymes. The invention further relates, use of compositions comprising such enzymes in cleaning processes and/or for deep cleaning of organic stains, methods for removal or reduction of components of organic matter.

### DESCRIPTION OF THE RELATED ART

Enzymes have been used in detergents for decades. Usually a cocktail of various enzymes is added to detergent compositions. The enzyme cocktail often comprises various enzymes, wherein each enzyme targets a specific substrate e.g. amylases are active towards starch stains, proteases on protein stains and so forth. Textiles surface and hard surfaces, such as dishes or the inner space of a laundry machine enduring a number of wash cycles, become soiled with many different types of soiling which may compose of proteins, grease, starch etc. One type of stains may be associated with organic matter such as stains from body soiling e.g. skin cell debris, sebum, sweat, biofilm, EPS, etc. Organic matter or stains may compose of different organic molecules such as polysaccharides, extracellular DNA (exDNA), starch and proteins. Organic matter may be poly-organic stains composing different organic stains. Some organic matter or stains composes an extracellular polymeric matrix, which may be sticky or glueing, which when present on textile, attracts soils and may course redeposition or backstaining of soil resulting in a greying of the textile. Additionally, organic matters or stains such as biofilms often cause malodor issue as various malodor molecules can be adhered by the polysaccharides, extracellular DNA (exDNA), and proteins in the complex extracellular matrix and be slowly released to cause consumer noticeable malodor issue. There is still a need for cleaning compositions, which effectively prevent, reduce or remove stains e.g. associated with poly-organic stains e.g. biofilm, such as starch, mannan, protein and DNA. The present invention provides new compositions fulfilling such need.

### Overview of Sequences

SEQ ID NO: 1 mature polypeptide obtained from Bacillus agaradhaerens

SEQ ID NO: 2 mature polypeptide obtained from Bacillus species.

SEQ ID NO: 3 mature polypeptide obtained from Bacillus species.

SEQ ID NO: 4 mature polypeptide obtained from Bacillus akibai.

SEQ ID NO: 5 mature polypeptide obtained from Bacillus mojavensis.

SEQ ID NO: 6 mature polypeptide obtained from Bacillus cibi

SEQ ID NO: 7 mature polypeptide obtained from Aspergillus oryzae

SEQ ID NO: 8 mature polypeptide obtained from Bacillus sp.

SEQ ID NO: 9 mature polypeptide obtained from Cytophaga sp.

SEQ ID NO: 10 mature polypeptide obtained from Bacillus sp.

SEQ ID NO: 11 mature polypeptide obtained from Bacillus sp.

SEQ ID NO: 12 mature polypeptide obtained from Bacillus subtilis

SEQ ID NO: 13 mature polypeptide obtained from Bacillus sp.

SEQ ID NO: 14 mature polypeptide obtained from Bacillus sp.

SEQ ID NO: 15 Artificial sequence

SEQ ID NO: 16 mature polypeptide obtained from Bacillus sp.

SEQ ID NO: 17 mature polypeptide obtained from Bacillus subtilis

SEQ ID NO: 18 mature polypeptide obtained from Humicola insolens

SEQ ID NO: 19 mature polypeptide obtained from Thielavia terrestris

SEQ ID NO: 20 mature polypeptide obtained from Thermomyces lanuginosus

SEQ ID NO: 21 mature polypeptide obtained from Bacillus bogoriensis

SEQ ID NO: 22 mature polypeptide obtained from Paenibacillus woosongensis

SEQ ID NO: 23 mature polypeptide obtained from Paenibacillus illinoisensis

SEQ ID NO: 24 mature polypeptide obtained from Neobulgaria sp.

SEQ ID NO: 25 mature polypeptide obtained from Preussia aemulans

SEQ ID NO: 26 mature polypeptide obtained from Yunnania penicillata

SEQ ID NO: 27 mature polypeptide obtained from Myrothecium roridum

SEQ ID NO: 28 mature polypeptide obtained from Chaetomium brasiliense

SEQ ID NO: 29 mature polypeptide obtained from Ascobolus stictoideus

SEQ ID NO: 30 mature polypeptide obtained from Chaetomium virescens

SEQ ID NO: 31 mature polypeptide obtained from Bacillus lentus

SEQ ID NO: 32 mature polypeptide obtained from Bacillus amyloliquefaciens

SEQ ID NO: 33 mature polypeptide obtained from Bacillus sp.

SEQ ID NO: 34 mature polypeptide obtained from Bacillus gibsonii

SEQ ID NO: 35 mature polypeptide obtained from Bacillus lentus

SEQ ID NO: 36 mature polypeptide obtained from Bacillus licheniformis

SEQ ID NO: 37 mature polypeptide obtained from Paenibacillus polymyxa

SEQ ID NO: 38 mature polypeptide obtained from Melanocarpus albomyces

SEQ ID NO: 39 mature polypeptide obtained from Paenibacillus species

SEQ ID NO: 40 mature polypeptide obtained from Bacillus hemicellulosilyticus

SEQ ID NO: 41 mature polypeptide obtained from Bacillus sp.

SEQ ID NO: 42 mature polypeptide obtained from Bacillus subtilis

### SUMMARY OF THE INVENTION

A first aspect of the invention relates to an enzyme composition, preferably a cleaning composition comprising a) a polypeptide having beta-glucanase activity and having at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the sequence selected from the group consisting of: SEQ ID NO: 1, SEQ ID NO: 2, SEQ ID NO: 3, SEQ ID NO: 4, SEQ ID NO: 5; and b) a polypeptide having DNase activity and having at least 60%, at least 65%, at least 70%, at least 75%, at least 80%, at least 85%, at least 90%, at least 95%, at least 98% or 100% sequence identity to the DNase shown in SEQ ID NO: 6 or SEQ ID NO: 7. The enzyme composition may optionally include at least one additional enzyme selected from the group consisting of amylase, cellulase, lipase, mannanase, pectinase, and protease or a combination thereof, and optionally includes at least one cleaning component.

A second aspect of the invention relates to the use of a composition, e.g., a cleaning composition comprising a) a polypeptide having beta-glucanase activity and having at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the sequence selected from the group consisting of: SEQ ID NO: 1, SEQ ID NO: 2, SEQ ID NO: 3, SEQ ID NO: 4, SEQ ID NO: 5; and b) a polypeptide having DNase activity and having at least 60%, at least 65%, at least 70%, at least 75%, at least 80%, at least 85%, at least 90%, at least 95%, at least 98% or 100% sequence identity to the DNase shown in SEQ ID NO: 6 or SEQ ID NO: 7. The enzyme composition may optionally include at least one additional enzyme selected from the group consisting of amylase, cellulase, lipase, mannanase, pectinase, and protease or a combination thereof, and at least one cleaning component for deep cleaning of an item, wherein the item is a textile or a surface.

A third aspect of the invention relates to a method of formulating a composition, such as a cleaning composition comprising a) a polypeptide having beta-glucanase activity and having at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the sequence selected from the group consisting of: SEQ ID NO: 1, SEQ ID NO: 2, SEQ ID NO: 3, SEQ ID NO: 4, SEQ ID NO: 5 and b) a polypeptide having DNase activity and having at least 60%, at least 65%, at least 70%, at least 75%, at least 80%, at least 85%, at least 90%, at least 95%, at least 98% or 100% sequence identity to the DNase shown in SEQ ID NO: 6 or SEQ ID NO: 7, and optionally at least one additional enzyme selected from the group consisting of amylase, cellulase, lipase, mannanase, pectinase, and protease or a combination thereof, and at least one cleaning component, comprising adding a beta-glucanase, a DNase, and optionally at least one additional enzyme and at least one cleaning component.

A fourth aspect of the invention relates to a kit intended for deep cleaning, wherein the kit comprises a solution of an enzyme mixture comprising a beta-glucanase, a DNase and optionally at least one additional enzyme selected from the group consisting of amylase, cellulase, lipase, mannanase, pectinase, and protease or a combination thereof. A fifth aspect a method of deep cleaning of an item, comprising the steps of:
a) contacting the item with a cleaning composition comprising i) a polypeptide having beta-glucanase activity and having at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the sequence selected from the group consisting of: SEQ ID NO: 1, SEQ ID NO: 2, SEQ ID NO: 3, SEQ ID NO: 4, SEQ ID NO: 5; and ii) a polypeptide having DNase having at least 60%, at least 65%, at least 70%, at least 75%, at least 80%, at least 85%, at least 90%, at least 95%, at least 98% or 100% sequence identity to the DNase shown in SEQ ID NO: 6 or SEQ ID NO: 7, and optionally at least one additional enzyme selected from the group consisting of amylase, cellulase, lipase, mannanase, pectinase, and protease or a combination thereof, and at least one cleaning component; and
b) optionally rinsing the item, wherein the item is preferably a textile.

### DETAILED DESCRIPTION OF THE INVENTION

### Definitions

Amylases (EC 3.2.1) are enzymes which catalyze the hydrolysis of starch, glycogen, and related polysaccharides to oligosaccharides, maltose, or glucose. Amylases are glycoside hydrolases and act on α-1,4-glycosidic bonds. The amylases suitable in the cleaning compositions of the invention are preferably alpha amylases. Alpha-amylases (EC 3.2.1.1) includes 1,4-α-D-glucan glucanohydrolase and glycogenase and are calcium metalloenzymes. By acting at random locations along the starch chain, alpha-amylase breaks down long-chain carbohydrates, ultimately yielding maltotriose and maltose from amylose, or maltose, glucose and “limit dextrin” from amylopectin. Suitable amylases of the present invention are preferably microbial e.g. obtained from bacterial or fungal sources. The term “alpha-amylase activity” means the activity of alpha 1,4-glucan 4 glucanohydrolases, E.C. 3.2.1.1, which constitute a group of enzymes, which catalyze hydrolysis of starch and other linear and branched 1,4 alpha-glucosidic oligo and poly-saccharides. Alpha-amylase activity may be determined by Assay IV as described in the Examples herein.

Beta-glucanase as used herein means an endo beta-1,4-glucanase activity (e.g. endo-1,4-β-D-glucanase) that catalyzes the hydrolyses of a beta-1,4-bonds connecting two glucosyl residues in a beta-glucan. Non-limiting examples of beta-glucanases as defined herein include cellulases (e.g. EC 3.2.1.4, e.g. having endo-cellulase activity on β-1,4 linkages between D-glucose units and licheninases (or lichenases) (e.g. EC 3.2.1.73) hydrolysing (1,4)-beta-D-glucosidic linkages in beta-D-glucans containing (1,3)- and (1,4)-bonds. Beta-glucanases (e.g. EC 3.2.1.4) can, for example, perform endohydrolysis of (1,4)-beta-D-glucosidic linkages in cellulose, lichenin and cereal beta-D-glucans and will also hydrolyze 1,4-linkages in beta-D-glucans containing 1,3-linkages. For purposes of the present invention, beta-glucanase activity is determined according to the procedure described in the Examples. In one aspect of the invention, the beta-glucanases useful according to the present invention have at least 20%, e.g., at least 40%, at least 50%, at least 60%, at least 70%, at least 80%, at least 90%, at least 95%, or at least 100% of the beta-glucanase activity of the polypeptide having the sequence selected from the group consisting of: SEQ ID NO: 1, SEQ ID NO: 2, SEQ ID NO: 3, SEQ ID NO: 4, SEQ ID NO: 5. Beta-glucanase activity can suitably be measured using barley beta-glucan as substrate. A preferred assay for determining beta-glucanase activity is disclosed in Assay I (AZCL-Barley beta-glucan assay). A further subgroup of beta-glucanases as defined herein, also known as a licheninases (or lichenases) (e.g. EC 3.2.1.73), can also be used to catalyse the hydrolysis of the beta-1,4-glucosidic bonds to give beta-glucans. Licheninases (or lichenases) (e.g. EC 3.2.1.73) hydrolyse (1,4)-beta-D-glucosidic linkages in beta-D-glucans containing (1,3)- and (1,4)-bonds and can act on lichenin and cereal beta-D-glucans, but not on beta-D-glucans containing only 1,3- or 1,4-bonds. As used herein the term “beta-glucanase activity” comprises licheninase (or lichenases) (e.g. EC 3.2.1.73) activity.

Beta-glucan as used herein means a polysaccharide that only contain glucose as structural components, and in which the glucose units are linked by beta-glycosidic bonds. Non-limiting examples of beta-glucans include beta-D-glucans, beta-1,3-1,4 glucans, mix-linkage beta-glucans, barley beta-glucans, oatmeal beta-glucans.

Biofilm is produced by any group of microorganisms in which cells stick to each other or stick to a surface, such as a textile, dishware or hard surface or another kind of surface. These adherent cells are frequently embedded within a self-produced matrix of extracellular polymeric substance (EPS). Biofilm EPS is a polymeric conglomeration generally composed of extracellular DNA, proteins, and polysaccharides. Biofilms may form on living or non-living surfaces. The microbial cells growing in a biofilm are physiologically distinct from planktonic cells of the same organism, which, by contrast, are single-cells that may float or swim in a liquid medium. Bacteria living in a biofilm usually have significantly different properties from planktonic bacteria of the same species, as the dense and protected environment of the film allows them to cooperate and interact in various ways. One benefit of this environment for the microorganisms is increased resistance to detergents and antibiotics, as the dense extracellular matrix and the outer layer of cells protect the interior of the community. On laundry biofilm producing bacteria can be found among the following species: Acinetobacter sp., Aeromicrobium sp., Brevundimonas sp., Microbacterium sp., Micrococcus luteus, Pseudomonas sp., Staphylococcus epidermidis, and Stenotrophomonas sp. On hard surfaces biofilm producing bacteria can be found among the following species: Acinetobactersp., Aeromicrobium sp., Brevundimonas sp., Microbacterium sp., Micrococcus luteus, Pseudomonas sp., Staphylococcus epidermidis, Staphylococcus aureus and Stenotrophomonas sp.

Cellulolytic enzyme or cellulase, “cellulolytic enzyme” or “cellulase” means one or more (e.g., several) enzymes that hydrolyze a cellulosic material. Such enzymes include endoglucanase(s), cellobiohydrolase(s), beta-glucosidase(s), or combinations thereof. The two basic approaches for measuring cellulolytic activity include: (1) measuring the total cellulolytic activity, and (2) measuring the individual cellulolytic activities (endoglucanases, cellobiohydrolases, and beta-glucosidases) as reviewed in Zhang et al., Outlook for cellulase improvement: Screening and selection strategies, 2006, Biotechnology Advances 24: 452-481. Total cellulolytic activity is usually measured using insoluble substrates, including Whatman N21 filter paper, microcrystalline cellulose, bacterial cellulose, algal cellulose, cotton, pretreated lignocellulose, etc. The most common total cellulolytic activity assay is the filter paper assay using Whatman N21 filter paper as the substrate. The assay was established by the International Union of Pure and Applied Chemistry (IUPAC) (Ghose, 1987, Measurement of cellulase activities, Pure Appl. Chem. 59: 257-68). Cellulases includes enzymes that catalyzes the hydrolysis of the 1,4-beta-D-glycosidic linkages in cellulose, hemicellulose, lichenin, and cereal beta-D-glucans. The term cellulase includes endo-1,4-beta-D-glucanase (beta-1,4-glucanase, beta-1,4-endoglucan hydrolase, endoglucanase D, 1,4-(1,3,1,4)-beta-D-glucan 4-glucanohydrolase) and carboxymethyl cellulase. Cellulases includes endo-cellulases (EC 3.2.1.4) which randomly cleave internal bonds at amorphous sites that create new chain ends and exocellulases (EC 3.2.1.91) that cleave two to four units from the ends of the exposed chains produced by endocellulase, resulting in tetrasaccharides or disaccharides, such as cellobiose. Exocellulases are further classified into type I, that work processively from the reducing end of the cellulose chain, and type II, that work processively from the nonreducing end. Suitable cellulases include complete cellulases or mono-component endoglucanases of bacterial or fungal origin. Chemically or genetically modified mutants are included. The cellulase may for example be a mono-component or a mixture of mono-component endo-1,4-beta-glucanase often just termed endoglucanases. Cellulases include enzymes having xyloglucanase activity Cellulase activity may be determined as described in Assay V in the Examples herein.

Cellulosic material the term “cellulosic material” means any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Cleaning component: The cleaning component e.g. the detergent adjunct ingredient is different to the DNase and additional enzymes. The precise nature of these additional cleaning components e.g. adjunct components, and levels of incorporation thereof, will depend on the physical form of the composition and the nature of the operation for which it is to be used. Suitable cleaning components e.g. adjunct materials include, but are not limited to the components described below such as surfactants, builders, flocculating aid, chelating agents, dye transfer inhibitors, enzymes, enzyme stabilizers, enzyme inhibitors, catalytic materials, bleach activators, hydrogen peroxide, sources of hydrogen peroxide, preformed peracids, polymeric agents, clay soil removal/anti-redeposition agents, brighteners, suds suppressors, dyes, perfumes, structure elasticizing agents, fabric softeners, carriers, hydrotropes, builders and co-builders, fabric huing agents, anti-foaming agents, dispersants, processing aids, and/or pigments.

Cleaning composition: The term “cleaning composition” refers to compositions that find use in the removal of undesired compounds from items to be cleaned, such as textiles and hard surfaces. The cleaning composition may be used to e.g. clean textiles or hard surfaces (e.g. dishes) for both household cleaning and industrial cleaning. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; fabric fresheners; fabric softeners; and textile and laundry pre-spotters/pretreatment). In addition to containing the DNase and additional enzymes of the invention, the cleaning composition may contain e.g. detergent adjunct ingredients such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers.

DNases are polypeptides with DNase (deoxyribonuclease) activity that catalyzes the hydrolytic cleavage of phosphodiester linkages in a DNA backbone, thus degrading DNA. Exodeoxyribonuclease cut or cleaves residues at the end of the DNA back bone where endo-deoxyribonucleases cleaves or cut within the DNA backbone. A DNase may cleave only double-stranded DNA or may cleave double stranded and single stranded DNA. The term “DNases” and the expression “a polypeptide with DNase activity” may be used interchangeably throughout the application. For purposes of the present invention, DNase activity is determined according to the procedure described in the Assay II or Assay III. Preferably the DNase is selected from any of the enzyme classes E.C.3.1, preferably E.C.3.1.21. Preferably, the polypeptide having DNase activity is obtained from a microorganism and the DNase is a microbial enzyme. The DNase is preferably of fungal or of bacterial origin.

By the term “deep cleaning” is meant reduction, disruption or removal of components, which may be comprised in organic matter, e.g. skin debris, dead cell material, sebum, sweat and biofilm, such as polysaccharides, grease, proteins, starch, DNA, soil or other components present in the organic matter. The organic matter may be termed poly-organic stains comprising more than one organic component such as starch, grease, protein, DNA and mannan.

The term “enzyme detergency benefit” is defined herein as the advantageous effect an enzyme may add to a detergent compared to the same detergent without the enzyme. Important detergency benefits which can be provided by enzymes are stain removal with no or very little visible soils after washing and/or cleaning, prevention or reduction of redeposition of soils released in the washing process (an effect that also is termed anti-redeposition), restoring fully or partly the whiteness of textiles which originally were white but after repeated use and wash have obtained a greyish or yellowish appearance (an effect that also is termed whitening). Textile care benefits, which are not directly related to catalytic stain removal or prevention of redeposition of soils, are also important for enzyme detergency benefits. Examples of such textile care benefits are prevention or reduction of dye transfer from one fabric to another fabric or another part of the same fabric (an effect that is also termed dye transfer inhibition or anti-backstaining), removal of protruding or broken fibers from a fabric surface to decrease pilling tendencies or remove already existing pills or fuzz (an effect that also is termed anti-pilling), improvement of the fabric-softness, colour clarification of the fabric and removal of particulate soils which are trapped in the fibers of the fabric or garment. Enzymatic bleaching is a further enzyme detergency benefit where the catalytic activity generally is used to catalyze the formation of bleaching components such as hydrogen peroxide or other peroxides. Textile care benefits, which are not directly related to catalytic stain removal or prevention of redeposition of soils, are also important for enzyme detergency benefits. Examples of such textile care benefits are prevention or reduction of dye transfer from one textile to another textile or another part of the same textile (an effect that is also termed dye transfer inhibition or anti-backstaining), removal of protruding or broken fibers from a textile surface to decrease pilling tendencies or remove already existing pills or fuzz (an effect that also is termed anti-pilling), improvement of the textile-softness, colour clarification of the textile and removal of particulate soils which are trapped in the fibers of the textile. Enzymatic bleaching is a further enzyme detergency benefit where the catalytic activity generally is used to catalyze the formation of bleaching component such as hydrogen peroxide or other peroxides or other bleaching species.”

The term “hard surface cleaning” is defined herein as cleaning of hard surfaces wherein hard surfaces may include floors, tables, walls, roofs etc. as well as surfaces of hard objects such as cars (car wash) and dishes (dish wash). Dish washing includes but are not limited to cleaning of plates, cups, glasses, bowls, cutlery such as spoons, knives, forks, serving utensils, ceramics, plastics, metals, china, glass and acrylics.

The term “laundering” relates to both household laundering and industrial laundering and means the process of treating textiles with a solution containing a cleaning or detergent composition of the present invention. The laundering process can for example be carried out using e.g. a household or an industrial washing machine or can be carried out by hand.

Lipases includes enzymes which catalyze the hydrolysis of fats (lipids). Lipases are a sub class of esterases. Lipases suitable in the present invention include phospholipases, acyltransferases or perhydrolases e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028). Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147). Lipase activity may be determined as described in Assay VI in the Examples herein.

By the term “malodor” is meant an odor which is not desired on clean items. One example of malodor is compounds with an unpleasant smell, which may be produced by microorganisms. Another example is unpleasant smells can be sweat or body odor adhered to an item which has been in contact with human or animal. Another example of malodor can be the odor from spices, which sticks to items for example curry or other exotic spices which smells strongly.

Mannanases include enzymes that catalyzes the hydrolysis of mannans, which is a highly branched polymer of mannose. The mannanases of the invention are preferably of microbial origin such as bacterial or fungal mannanases. The mannanase preferably having mannan endo-1,4-beta-mannosidase activity (EC 3.2.1.78) that catalyzes the hydrolysis of 1,4-β-D-mannosidic linkages in mannans, galactomannans and/or glucomannans. The mannanase may be a GH5 mannanase such as an endo-1,4-β-Mannanase or a GH26 endo-1,4 β-Mannanase. Mannanase activity may be determined as described in Assay VII in the Examples herein.

The term “mature polypeptide” means a polypeptide in its final form following translation and any post-translational modifications, such as N-terminal processing, C-terminal truncation, glycosylation, phosphorylation, etc.

“Pectinase” denotes a pectinase enzyme defined according to the art where pectinases are a group of enzymes that cleave glycosidic linkages of pectic substances mainly poly(1,4-alpha-D-galacturonide and its derivatives (see reference Sakai et al., Pectin, pectinase and protopectinase: production, properties and applications, pp 213-294 in: Advances in Applied Microbiology vol:39, 1993). Preferably a pectinase is a pectinase enzyme which catalyzes the random cleavage of alpha-1,4-glycosidic linkages in pectic acid also called polygalacturonic acid by transelimination such as the enzyme class polygalacturonate lyase (EC 4.2.2.2) (PGL) also known as poly(1,4-alpha-D-galacturonide) lyase also known as pectate lyase.

Proteases includes enzymes that hydrolyze peptide bonds and the term incudes peptidase and proteinase. Serine proteases (or serine endopeptidases), E.C. 3.4.21 are enzymes that cleave peptide bonds in proteins, in which serine serves as the nucleophilic amino acid at the active site. Suitable proteases include those of bacterial, fungal, plant, viral or animal origin e.g. vegetable or microbial origin. Microbial origin is preferred. Chemically modified or protein engineered mutants are included. Most relevant proteases for laundry may be the alkaline proteases, such as a serine protease. A serine protease may for example be of the S1 family, such as trypsin, or the S8 family such as subtilisin. A metalloproteases protease may for example be a thermolysin from e.g. family M4 or other metalloproteases such as those from M5, M7 or M8 families. The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family. Protease activity may be determined as described in Assay VIII in the Examples herein.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment).

The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, cellulose acetate fibers (tricell), lyocell or blends thereof. The textile or fabric may also be non-cellulose based such as natural polyamides including wool, camel, cashmere, mohair, rabbit and silk or synthetic polymers such as nylon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g. polyamide fiber, acrylic fiber, polyester fiber, polyvinyl chloride fiber, polyurethane fiber, polyurea fiber, aramid fiber), and/or cellulose-containing fiber (e.g. rayon/viscose, ramie, flax/linen, jute, cellulose acetate fiber, lyocell). Fabric may be conventional washable laundry, for example stained household laundry. When the term fabric or garment is used, it is intended to include the broader term textiles as well.

The term “variant” means a polypeptide having the activity of the parent or precursor polypeptide and comprising an alteration, i.e., a substitution, insertion, and/or deletion, at one or more (e.g., several) positions compared to the precursor or parent polypeptide. A substitution means replacement of the amino acid occupying a position with a different amino acid; a deletion means removal of the amino acid occupying a position; and an insertion means adding an amino acid adjacent to and immediately following the amino acid occupying a position.

The term “wash performance” is used as an enzyme's ability to remove stains present on the object to be cleaned during e.g. wash or hard surface cleaning.

The term “whiteness” is defined herein as a greying, yellowing of a textile. Loss of whiteness may be due to removal of optical brighteners/hueing agents. Greying and yellowing can be due to soil redeposition, body soils, colouring from e.g. iron and copper ions or dye transfer. Whiteness might include one or several issues from the list below: colourant or dye effects; incomplete stain removal (e.g. body soils, sebum etc.); redeposition (greying, yellowing or other discolourations of the object) (removed soils reassociate with other parts of textile, soiled or unsoiled); chemical changes in textile during application; and clarification or brightening of colours.

### Nomenclature

For purposes of the present invention, the nomenclature “E/Q” or EQ means that the amino acid at a given position may be a glutamic acid (Glu, E) or a glutamine (Gln, Q). Likewise, the nomenclature “V/G/A/I” or VGAI means that the amino acid at this position may be a valine (Val, V), glycine (Gly, G), alanine (Ala, A) or isoleucine (Ile, I), and so forth for other combinations as described herein. Unless otherwise limited further, the amino acid X is defined such that it may be any of the 20 natural amino acids.

Substitutions are typically indicated with the original amino acid (the amino acid present in a parent sequence), the position number, and the replacement amino acid. For example, A226V indicates that the alanine residue in position 226 has been replaced by a valine residue. Different parent enzymes may have different amino acids in the position corresponding to position 226 of the parent sequence. Thus, A226V is not limited to substitutions of alanine. Any amino acid may be replaced in the substitution, which may also be indicated with an X as X226V. Both annotations may be used interchangeably. The substitution A226V, may also be written e.g. enzyme X comprising valine at a position corresponding to position 226 of SEQ ID NO XX.

Deletions are indicated with an asterisk (*). For example, G184* indicates that the original glycine residue in position 184 has been deleted.

Insertions are indicated by listing the original amino acid, the position number, the original amino acid and the inserted amino acid. For example, S97SD indicates that an aspartic acid residue has been inserted after the serine residue in position 97.

Multiple alternative mutations may be separated by commas e.g. T51I, S52Q, N54K, meaning that one or more maybe all of the listed mutations may be present. Thus, meaning the above example that either one, two or three (thus all) of the mutations may present compared to the parent enzyme.

When mutations in alteration sets are separated by commas this means that all the alterations in the set are present and the selection is between the lists of alterations (alteration sets). E.g. “comprising one of the alterations sets selected from the group consisting of
a. H1*, D183*, G184*, N195F, V206Y;
b. H1*, D183*, G184*, N195F, M202L, V206L, R320K, R458K; . . .
   means the selection is made between a., b. etc. of the alteration sets. Thus, a set is then a list of alterations which is all present though separated by commas.

SEQ ID NO XX+mutation(s) is to be understood as variants of an enzyme parent comprising specified mutations compared to the specific parent sequence.

The term “corresponding to” reflects the numbering system used and that various starting proteases (parent proteases) may have different length. Thus, a given starting amylase is aligned with e.g. SEQ ID NO: 8 and the position corresponding to e.g. pos 140 is determined.

The terms parent enzyme includes terms such as reference enzyme, back bone or starting enzyme and is used to denote the enzyme into which to mutations e.g. substitutions are made. The terms may be used interchangeably

### Compositions

The invention relates to enzyme compositions such as cleaning compositions comprising a beta-glucanase and a DNase. Preferably, the composition is a cleaning composition and the beta-glucanase provides at least one enzyme detergency benefit as described in “definitions” in combination with one or more cleaning composition component. The beta-glucanase and the DNase preferably act synergistically to remove components such as skin debris, dead cells, components of biofilm, biofilm EPS and other organic components from e.g. poly-organic stains. Optionally, the enzyme composition further includes an additional cleaning enzyme e.g. an additional enzyme providing enzyme detergency benefit.

One embodiment of the invention relates to a composition, such as a cleaning composition comprising a beta-glucanase and a DNase. Optionally, the enzyme composition further comprises an additional cleaning enzyme selected from the group consisting of an amylase, a cellulase, a lipase, a mannanase, a pectinase, and a protease or a combination thereof, and at least one cleaning component.

One embodiment relates to a cleaning composition comprising a) a polypeptide having beta-glucanase activity and having at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the sequence selected from the group consisting of: SEQ ID NO: 1, SEQ ID NO: 2, SEQ ID NO: 3, SEQ ID NO: 4, SEQ ID NO: 5 and b) a polypeptide having DNase activity and having at least 60%, at least 65%, at least 70%, at least 75%, at least 80%, at least 85%, at least 90%, at least 95%, at least 98% or 100% sequence identity to the DNase shown in SEQ ID NO: 6 or SEQ ID NO: 7. In an embodiment, the beta-glucanase provides at least one enzyme detergency benefit.

In a further embodiment, the cleaning composition comprises at least one additional enzyme selected from the group consisting of amylase, cellulase, lipase, mannanase and protease or a combination thereof, and at least one cleaning component, wherein the amylase, cellulase, lipase, mannanase, pectinase, and protease or combination thereof provide at least one enzyme detergency benefit.

Useful beta-glucanases are those of microbial origin, for example of fungal or bacterial origin.

Particularly useful DNases may be those of microbial origin. In one embodiment, the cleaning composition comprise a DNase from bacteria.

One embodiment of the invention relates to a cleaning composition comprising a beta-glucanase and a DNase, wherein the DNase is obtained from Bacillus, preferably Bacillus cibi. One embodiment of the invention relates to a cleaning composition comprising a beta-glucanase and a DNase, wherein the DNase is obtained from Aspergillus, preferably Aspergillus oryzae.

The term “obtained from” as used herein in connection with a given source shall mean that the enzyme of the invention is produced by the source or by a strain in which the polynucleotide encoding the enzyme of the invention from the source has been inserted. In one aspect, the enzyme obtained from a given source is secreted extracellularly.

One embodiment of the invention relates to a composition, such as a cleaning composition comprising a beta-glucanase having at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, and a DNase having at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7.

One embodiment of the invention relates to a composition, such as a cleaning composition comprising a beta-glucanase having at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 2, and a DNase having at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7.

One embodiment of the invention relates to a composition, such as a cleaning composition comprising a beta-glucanase having at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 3, and a DNase having at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7.

One embodiment of the invention relates to a composition, such as a cleaning composition comprising a beta-glucanase having at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 4, and a DNase having at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7.

One embodiment of the invention relates to a composition, such as a cleaning composition comprising a beta-glucanase having at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 5, and a DNase having at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7.

One embodiment of the invention relates to an enzyme composition, e.g. a cleaning composition comprising a beta-glucanase, a DNase, a cleaning enzyme selected from an amylase, a cellulase, a lipase, a mannanase, a pectinase, and a protease and at least one cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7. The term cleaning enzyme means an enzyme with cleaning benefits e.g. enzyme detergency benefit.

One embodiment of the invention relates to an enzyme composition, e.g. a cleaning composition comprising a beta-glucanase, a DNase, a cleaning enzyme selected from an amylase, a cellulase, a lipase, a mannanase, a pectinase, and a protease and at least one cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 2, the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7.

One embodiment of the invention relates to an enzyme composition, e.g. a cleaning composition comprising a beta-glucanase, a DNase, a cleaning enzyme selected from an amylase, a cellulase, a lipase, a mannanase, a pectinase, and a protease and at least one cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 3, the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7.

One embodiment of the invention relates to an enzyme composition, e.g. a cleaning composition comprising a beta-glucanase, a DNase, a cleaning enzyme selected from an amylase, a cellulase, a lipase, a mannanase, a pectinase, and a protease and at least one cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 4, the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7.

One embodiment of the invention relates to an enzyme composition, e.g. a cleaning composition comprising a beta-glucanase, a DNase, a cleaning enzyme selected from an amylase, a cellulase, a lipase, a mannanase, a pectinase and a protease and at least one cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 5, the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7.

One preferred cleaning enzyme to be combined with the beta-glucanase and the DNase enzyme is an amylase in particular an alpha-amylase (alpha-1,4-glucan-4-glucanohydrolases, E.C. 3.2.1.1). Amylases catalyzes hydrolysis of starch and other linear and branched 1,4-gluosidic oligo- and polysaccharides. Amylases have several applications such as detergent, baking, brewing, starch liquefaction and saccharification e.g. in preparation of high fructose syrups or as part of ethanol production from starch. An alpha-amylase useful in a cleaning composition of the invention is preferably an enzyme classified under EC 3.2.1.1. One embodiment relates to a cleaning composition comprising a beta-glucanase, a DNase, an amylase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the amylase is classified under EC 3.2.1.1. The alpha-amylase may be bacterial or fungal. A bacterial alpha-amylase to be used in a composition according to the invention may, e.g., be derived from a strain of the genus Bacillus, which is sometimes also referred to as the genus Geobacillus. In an embodiment the Bacillus alpha-amylase is derived from a strain of B. amyloliquefaciens, B. licheniformis, B. stearothermophilus, B. halmapalus, or B. subtilis, but may also be derived from another Bacillus sp. e.g. Bacillus TS-23 is described in WO2014/195356. The amylases may also be obtained from bacteria such as Cytophaga.

One embodiment relates to a cleaning composition comprising a beta-glucanase, a DNase, an amylase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the amylase is obtained from Bacillus e.g. B. amyloliquefaciens, B. licheniformis, B. stearothermophilus, B. halmapalus, or B. subtilis, Bacillus TS-23 or from Cytophaga.

One embodiment relates to a cleaning composition comprising a beta-glucanase, a DNase, an amylase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the amylase is selected from the group consisting of;
a) an amylase having at least 60%, at least 65%, at least 70%, at least 75%, at least 80%, at least 85%, at least 90%, at least 95%, at least 98% or 100% sequence identity to SEQ ID NO: 8 or SEQ ID NO: 41, or an amylase having at least 60%, at least 65%, at least 70%, at least 75%, at least 80%, at least 85%, at least 90%, at least 95%, at least 98% but less than 100% sequence identity to SEQ ID NO: 8 or SEQ ID NO 35 comprising a two amino acid deletion in the sequence region R180, S181, T182, G183, compared to SEQ ID NO: 8, wherein each position corresponds to the position in SEQ ID NO: 8;
b) an amylase having at least 60%, at least 65%, at least 70%, at least 75%, at least 80%, at least 85%, at least 90%, at least 95%, at least 98% or 100% sequence identity to SEQ ID NO: 8 or SEQ ID NO: 41, or an amylase having at least 60%, at least 65%, at least 70%, at least 75%, at least 80%, at least 85%, at least 90%, at least 95%, at least 98% but less than 100% sequence identity to SEQ ID NO: 8 or SEQ ID NO: 41 comprising one of the alterations set selected from the group consisting of:
   a. R180*, S181*, S243Q, G475K;
   b. R180*, T182*, S243Q, G475K;
   c. R180*, T182*, G183S, S243Q, G475K; and
   d. R180*, S181*, Y242F, S243Q, F266Y, G475K compared to SEQ ID NO: 8, wherein each position corresponds to the position in SEQ ID NO: 8;
c) an amylase having at least 60%, at least 65%, at least 70%, at least 75%, at least 80%, at least 85%, at least 90%, at least 95%, at least 98% or 100% sequence identity to SEQ ID NO: 9, or an amylase having at least 60%, at least 65%, at least 70%, at least 75%, at least 80%, at least 85%, at least 90%, at least 95%, at least 98% but less than 100% sequence identity to SEQ ID NO: 9, comprising a two amino acid deletion in the sequence region R178, G179, T180, G181 compared to SEQ ID NO: 9, wherein each position corresponds to the position in SEQ ID NO: 9;
d) an amylase having at least 60%, at least 65%, at least 70%, at least 75%, at least 80%, at least 85%, at least 90%, at least 95%, at least 98% or 100% sequence identity to SEQ ID NO: 9, or an amylase having at least 60%, at least 65%, at least 70%, at least 75%, at least 80%, at least 85%, at least 90%, at least 95%, at least 98% but less than 100% sequence identity to SEQ ID NO: 9, comprising one of the alterations set selected from the group consisting of:
   I. R178*, G179*, E187P, I203Y, G476K;
   II. R178*, G179*, E187P, M199L, I203Y, G476K;
   III. R178*, G179*, E187P, I203Y R458N, T459S, D460T, G476K;
   IV. N126Y, F153W, R178*, G179*, T180H, I203Y, S241Q;
   V. N126Y, F153W, R178*, G179*, T180H, I203Y, S241Q, S362A, R377Y;
   VI. T38N, N126Y, T1291, F153W, R178*, G179*, T180D, E187P, I203Y, G476K, G477E; and
   VII. N126Y, F153W, R178*, G179*, T180H, E187P, I203Y, S241Q, G476K, G477E, compared to SEQ ID NO 3, wherein each position corresponds to the position in SEQ ID NO: 9;
e) an amylase having at least 60%, at least 65%, at least 70%, at least 75%, at least 80%, at least 85%, at least 90%, at least 95%, at least 98% or 100% sequence identity to SEQ ID NO: 10, or an amylase having at least 60%, at least 65%, at least 70%, at least 75%, at least 80%, at least 85%, at least 90%, at least 95%, at least 98% but less than 100% sequence identity to SEQ ID NO: 10, comprising a two amino acid deletion in the sequence region R181, G182, D183, G184 compared to SEQ ID NO: 10, wherein each position corresponds to the position in SEQ ID NO: 10;
f) an amylase having at least 60%, at least 65%, at least 70%, at least 75%, at least 80%, at least 85%, at least 90%, at least 95%, at least 98% or 100% sequence identity to SEQ ID NO: 10, or an amylase having at least 60%, at least 65%, at least 70%, at least 75%, at least 80%, at least 85%, at least 90%, at least 95%, at least 98% but less than 100% sequence identity to SEQ ID NO: 10 comprising an alteration at one or more, preferably at all of the position(s) selected from 3, 4, 5, 74, 118, 167, 170, 177, 195, 202, 204, 271, 320, 330, 377, 385, 445, 458, 475, 476, 314, 315 or 316, compared to SEQ ID NO: 10, wherein each position corresponds to the position in SEQ ID NO: 10;
g) an amylase having at least 60%, at least 65%, at least 70%, at least 75%, at least 80%, at least 85%, at least 90%, at least 95%, at least 98% or 100% sequence identity to SEQ ID NO: 11, or an amylase having at least 60%, at least 65%, at least 70%, at least 75%, at least 80%, at least 85%, at least 90%, at least 95%, at least 98% but less than 100% sequence identity to SEQ ID NO: 11 preferably comprising a two amino acid deletion in the sequence region R181, G182, D183, G184, compared to SEQ ID NO 5, wherein each position corresponds to the position in SEQ ID NO: 11;
h) an amylase having at least 60%, at least 65%, at least 70%, at least 75%, at least 80%, at least 85%, at least 90%, at least 95%, at least 98% or 100% sequence identity to SEQ ID NO: 11, or an amylase having at least 60%, at least 65%, at least 70%, at least 75%, at least 80%, at least 85%, at least 90%, at least 95%, at least 98% but less than 100% sequence identity to SEQ ID NO: 11 comprising one of the alterations set selected from the group consisting of
   a. D183*, G184*, N195F, Y243F;
   b. D183*, G184*, N195F, V206Y, Y243F;
   c. W140Y, D183*, G184*, N195F, V206Y, Y243F, E260G, G304R, G476K;
   d. W140Y, D183*, G184*, N195F, V206Y, Y243F, E260G, G477E;
   e. W140Y, D183*, G184*, N195F, V206Y, Y243F, W284D;
   f. W140Y, N195F, V206Y, Y243F, E260G, G477E;
   g. G109A, W140Y, N195F, V206Y, Y243F, E260G;
   h. T51I, S52Q, N54K, G109A, W140Y, N195F, V206Y, Y243F, E260G, G476E;
   i. W140Y, N195F, V206Y, Y243F, E260G, W284R, G477K;
   j. W140Y, N195F, V206Y, Y243F, E260G, W284F, G477R; and
   k. H1*, G7A, G109A, W140Y, D183*, G184*, N195F, V206Y, Y243F, E260G, N280S, G304R, E391A, G476K, compared to SEQ ID NO: 11, wherein each position corresponds to the position in SEQ ID NO: 11;
i) an amylase having at least 60%, at least 65%, at least 70%, at least 75%, at least 80%, at least 85%, at least 90%, at least 95%, at least 98% or 100% sequence identity to SEQ ID NO: 12, or an amylase having at least 60%, at least 65%, at least 70%, at least 75%, at least 80%, at least 85%, at least 90%, at least 95%, at least 98% but less than 100% sequence identity to SEQ ID NO: 12, comprising a two amino acid deletion in the sequence region R181, G182, D183, G184, compared to SEQ ID NO: 12, wherein each position corresponds to the position in SEQ ID NO: 12;
j) an amylase having at least 60%, at least 65%, at least 70%, at least 75%, at least 80%, at least 85%, at least 90%, at least 95%, at least 98% or 100% sequence identity to SEQ ID NO: 12, or an amylase having at least 60%, at least 65%, at least 70%, at least 75%, at least 80%, at least 85%, at least 90%, at least 95%, at least 98% but less than 100% sequence identity to SEQ ID NO: 12, comprising one of the alterations set selected from the group consisting of
   I. R118K, D183*, G184*, N195F, R320K, R458K;
   II. M9I, D183*, G184*, R118K, N195F, M202L, R320K, M323T, R458K;
   III. M9L, G149A, R118K, G182T, D183*, G184*, G186A, N195F, M202L, T257I, Y295F, N299Y, M323T, A339S, E345R, R458K;
   IV. M9L, G149A, R118K, G182T, D183*, G184*, G186A, N195F, T246V, T257I, Y295F, N299Y, M323T, A339S, E345R, R458K; and
   V. M9L, G149A, G182T, D183*, G184*, G186A, M202L, T257I, Y295F, N299Y, M323T, A339S, E345R, N471E, compared to SEQ ID NO: 12, wherein each position corresponds to the position in SEQ ID NO: 12;
k) an amylase having at least 60%, at least 65%, at least 70%, at least 75%, at least 80%, at least 85%, at least 90%, at least 95%, at least 98% or 100% sequence identity to SEQ ID NO: 13, or an amylase having at least 60%, at least 65%, at least 70%, at least 75%, at least 80%, at least 85%, at least 90%, at least 95%, at least 98% but less than 100% sequence identity to SEQ ID NO: 13, comprising a two amino acid deletion in the sequence region R181, G182, D183, G184, compared to SEQ ID NO: 13, wherein each position corresponds to the position in SEQ ID NO: 13;
l) an amylase having at least 60%, at least 65%, at least 70%, at least 75%, at least 80%, at least 85%, at least 90%, at least 95%, at least 98% or 100% sequence identity to SEQ ID NO: 13, or an amylase having at least 60%, at least 65%, at least 70%, at least 75%, at least 80%, at least 85%, at least 90%, at least 95%, at least 98% but less than 100% sequence identity to SEQ ID NO: 13, comprising one of the alterations set selected from the group consisting of
   a. D183*, G184*, N195F, V206Y, R320K, R458K;
   b. D183*, G184*, N195F, M202L, V206L, R320K, R458K;
   c. G149A, G182T, D183*, G184*, N195F, M202L, V206L, T257I, Y295F, Q299Y, A339S, Q345R, R458K;
   d. G149A, G182T, D183*, G184*, N195F, V206L, M246V, T257I, Y295F, Q299Y, A339S, Q345R, R458K;
   e. G149A, G182T, D183*, G184*, M202L, V206L, T257I, Y295F, Q299Y, A339S, Q345R, H471E; and
   f. H1A, N54S, V56T, K72R, G109A, F113Q, R116Q, W167F, Q172G, A174S, G182*, D183*, G184T, N195F, V206L, K391A, F473R, G476K, compared to SEQ ID NO 7, wherein each position corresponds to the position in SEQ ID NO: 13;
m) an amylase having at least 60%, at least 65%, at least 70%, at least 75%, at least 80%, at least 85%, at least 90%, at least 95%, at least 98% or 100% sequence identity to SEQ ID NO: 14, or an amylase having at least 60%, at least 65%, at least 70%, at least 75%, at least 80%, at least 85%, at least 90%, at least 95%, at least 98% but less than 100% sequence identity to SEQ ID NO: 14, comprising a two amino acid deletion in the sequence region R181, G182, H183, G184, compared to SEQ ID NO: 14, wherein each position corresponds to the position in SEQ ID NO: 14;
n) an amylase having at least 60%, at least 65%, at least 70%, at least 75%, at least 80%, at least 85%, at least 90%, at least 95%, at least 98% or 100% sequence identity to SEQ ID NO: 14, or an amylase having at least 60%, at least 65%, at least 70%, at least 75%, at least 80%, at least 85%, at least 90%, at least 95%, at least 98% but less than 100% sequence identity to SEQ ID NO: 14, comprising one of the alterations set selected from the group consisting of
   a. H183*, G184*, 1405L, A421H, A422P, A428T;
   b. R118K, H183*, G184*, N195F, R320K, R458K;
   c. M9I, H183*, G184*, R118K, N195F, M202L, R320K, S323T, R458K;
   d. M9L, G149A, R118K, G182T, H183*, G184*, N195F, M202L, T257I, Y295F, N299Y, A339S, E345R, R458K;
   e. M9L, G149A, R118K, G182T, H183*, G184*, N195F, T246V, T257I, Y295F, N299Y, A339S, E345R, R458K; and
   f. M9L, G149A, G182T, H183*, G184*, M202L, T257I, Y295F, N299Y, S323T, A339S, E345R, compared to SEQ ID NO: 14, wherein each position corresponds to the position in SEQ ID NO: 14;
o) an amylase having at least 60%, at least 65%, at least 70%, at least 75%, at least 80%, at least 85%, at least 90%, at least 95%, at least 98% or 100% sequence identity to SEQ ID NO: 15, or an amylase having at least 60%, at least 65%, at least 70%, at least 75%, at least 80%, at least 85%, at least 90%, at least 95%, at least 98% but less than 100% sequence identity to SEQ ID NO: 15, comprising a two amino acid deletion in the sequence region R181, G182, G182, D183, compared to SEQ ID NO: 15, wherein each position corresponds to the position in SEQ ID NO: 15;
p) an amylase having at least 60%, at least 65%, at least 70%, at least 75%, at least 80%, at least 85%, at least 90%, at least 95%, at least 98% or 100% sequence identity to SEQ ID NO: 15, or an amylase having at least 60%, at least 65%, at least 70%, at least 75%, at least 80%, at least 85%, at least 90%, at least 95%, at least 98% but less than 100% sequence identity to SEQ ID NO: 15, comprising one of the alterations set selected from the group consisting of
   a. H1*, D183*, G184*, N195F, V206Y;
   b. H1*, D183*, G184*, N195F, M202L, V206L, R320K, R458K;
   c. G149A, G182T, D183*, G184*, N195F, M202L, V206L, T257I, Y295F, Q299Y, A339S, Q345R, R458K;
   d. G149A, G182T, D183*, G184*, N195F, V206L, M246V, T257I, Y295F, Q299Y, A339S, Q345R, R458K;
   e. G149A, G182T, D183*, G184*, M202L, V206L, T257I, Y295F, Q299Y, A339S, Q345R,
   f. H1*, N54S, V56T, G109A, Q169E, Q172K, A174*, G182*, D183*, N195F, V206L, K391A, G476K;
   g. G182*, D183*, N195F, W140Y, N260G, 5304R, R320A, G476K, V410I, V429I, F451W, C474V;
   h. H1*, N54S, V56T, K72R, G109A, F113Q, R116Q, W167F, Q172G, A174S, G182*, D183*, G184T, N195F, V206L, K391A, P473R, G476K;
   i. H1*, N54S, V56T, G109A, Q169E, Q172K, A174*, G182*, D183*, N195F, V206L, K391A, G476K;
   j. H1*, N54S, V56T, G109A, R116H, A174S, G182*, D183*, N195F, V206L, K391A, G476K;
   k. H1*, N54S, V56T, K72R, G109A, F113Q, R116Q, W167F, Q172G, A174S, G182*, D183*, G184T, N195F, V206L, K391A, P473R, G476K;
   l. H1*, N54S, V56T, G109A, F113Q, R116Q, Q172N, A174S, G182*, D183*, N195F, V206L, A265G, K391A, P473R, G476K;
   m. H1*, N54S, V56T, K72R, G109A, F113Q, W167F, Q172R, A174S, G182*, D183*, N195F, V206L, K391A, G476K;
   n. H1*, N54S, V56T, K72R, G109A, R116H, T134E, W167F, Q172G, L173V, A174S, G182*, D183*, N195F, V206L, G255A, K391A, G476K;
   o. H1*, N54S, V56T, K72R, G109A, R116H, T134E, W167F, Q172G, L173V, A174S, G182*, D183*, N195F, V206L, G255A, K391A, Q395P, T444Q, P473R, G476K;
   p. H1*, N54S, V56T, G109A, T134E, A174S, G182*, D183*, N195F, V206L, K391A, G476K;
   q. H1*, N54S, V56T, K72R, G109A, A174S, G182*, D183*, N195F, V206L, G255A, K391A, G476K;
   r. H1*, N54S, V56T, G109A, W167F, Q172E, L173P, A174K, G182*, D183*, N195F, V206L, K391A, G476K;
   s. H1*, N54S, V56T, G109A, R116Q, V120L, Q172G, L173V, A174S, G182*, D183*, G184T, N195F, V206L, A422P; and
   t. H1*, N54S, V56T, G109A, F113Q, R116Q, W167F, Q172G, 1173V, A174S, G182*, D183*, G184T, N195F, V206L, A422P compared to SEQ ID NO: 15, wherein each position corresponds to the position in SEQ ID NO: 15.

One preferred cleaning enzyme to be combined with the beta-glucanase and the DNase enzyme is an additional cellulase in particular an enzyme exhibiting endo-beta-1,4-glucanase activity. Cellulose is a polymer of glucose linked by beta-1,4-glucosidic bonds. Cellulose chains form numerous intra- and intermolecular hydrogen bonds, which results in the formation of insoluble cellulose micro-fibrils. Microbial hydrolysis of cellulose to glucose involve three major classes of cellulases: (i) endo-glucanases (EC 3.2.1.4) which cleave beta-1,4-glucosidic links randomly throughout cellulose molecules, also called endo-beta-1,4-glucanases; (ii) cellobiohydrolases (EC 3.2.1.91) which digest cellulose from the non-reducing end, releasing cellobiose; and (iii) beta-glucosidases (EC 3.2.1.21) which hydrolyse cellobiose and low molecular-weight cellodextrins to release glucose. The cellulases useful in the cleaning composition of the invention are preferably endo-glucanases (EC 3.2.1.4). Beta-1,4-glucosidic bonds are also present beta-glucans from plants such as barley and oats. In some cases, endo-glucanases also provide hydrolysis of such non-cellulose polymers. The cellulases are placed into different families of glycosyl hydrolases; fungal and bacterial glycosyl hydrolases have been grouped into 35 families (Henrissat, B.: A classification of glycosyl hydrolases based on amino acid sequence similarities. Biochem. J. 280 (1991), 309-316. Henrissat, B., and Bairoch, A.: New families in the classification of glycosyl hydrolases based on amino acid sequence similarities. Biochem. J. 293 (1993), 781-788.). Most cellulases comprises or consists of a cellulose-binding domain (CBD) and a catalytic domain (CAD) separated by a linker which may be rich in proline and hydroxy amino acid residues. Another classification of cellulases has been established on the basis of the similarity of their CBDs (Gilkes et al. (1991)) giving five families of glycosyl hydrolases (I-V). Cellulases are synthesized by a large number of microorganisms which include fungi, actinomycetes, myxobacteria and true bacteria but also by plants. Especially endo-beta-1,4-glucanases of a wide variety of specificities have been identified. Many bacterial endo-glucanases have been described (Gilbert, H. J. and Hazlewood, G. P. (1993) J. Gen. Microbiol. 139:187-194. Henrissat, B., and Bairoch, A.: New families in the classification of glycosyl hydrolases based on amino acid sequence similarities. Biochem. J. 293 (1993), 781-788.). One preferred cellulase includes endo-beta-1,4-glucanase activity (EC 3.2.1.4), preferably obtained from Bacillus sp. AA349 (DSM 12648), as described in WO2002/099091. Other cellulases that are endo-beta-1,4-glucanase enzyme, includes the cellulase shown in SEQ ID NO: 16. Other preferred cellulases include those described in WO1996/029397, which discloses family 45 endoglucanases e.g. cellulases from Thielavia in particular a strain of Thielavia terrestris and the cellulases described in WO1991/017243, which discloses endoglucanases from e.g. of Humicola such as Humicola insolens.

Suitable cellulases include those from the genera Bacillus, Pseudomonas, Humicola, Myceliophthora, Fusarium, Thielavia, Trichoderma, and Acremonium. Exemplary cellulases include a fungal cellulase from Humicola insolens (U.S. Pat. No. 4,435,307) or from Trichoderma, e.g. T. reesei or T. viride. Other suitable cellulases are from Thielavia e.g. Thielavia terrestris as described in WO 96/29397 or the fungal cellulases produced from Myceliophthora thermophila and Fusarium oxysporum disclosed in U.S. Pat. Nos. 5,648,263, 5,691,178, 5,776,757, WO 89/09259 and WO 91/17244. Also relevant are cellulases from Bacillus as described in WO 02/099091 and JP 2000210081. Suitable cellulases are alkaline or neutral cellulases having care benefits. Examples of cellulases are described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307.

Cellulases includes a family 44 xyloglucanase, which a xyloglucanase enzyme such as the xyloglucanase shown in SEQ ID NO: 37.

One embodiment relates to a cleaning composition comprising a beta-glucanase, a DNase, a cellulase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the cellulase belongs to (EC 3.2.1.4), (EC 3.2.1.91) or (EC 3.2.1.21).

One embodiment relates to a cleaning composition comprising a beta-glucanase, a DNase, an additional cellulase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the cellulase is obtained from Thielavia, Humicola, Paenibacillus or Melanocarpus preferably Thielavia terrestris, Humicola insolens, Paenibacillus polymyxa or Melanocarpus albomyces

One embodiment relates to a cleaning composition comprising a beta-glucanase, a DNase, an additional cellulase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the cellulase is selected from the group consisting of;
a) a cellulase having at least 60%, at least 65%, at least 70%, at least 75%, at least 80%, at least 85%, at least 90%, at least 95%, at least 98% or 100% sequence identity to SEQ ID NO: 16;
b) a cellulase having at least 60%, at least 65%, at least 70%, at least 75%, at least 80%, at least 85%, at least 90%, at least 95%, at least 98% or 100% sequence identity to SEQ ID NO: 17;
c) a cellulase having at least 60%, at least 65%, at least 70%, at least 75%, at least 80%, at least 85%, at least 90%, at least 95%, at least 98% or 100% sequence identity to SEQ ID NO: 18;
d) a cellulase having at least 60%, at least 65%, at least 70%, at least 75%, at least 80%, at least 85%, at least 90%, at least 95%, at least 98% or 100% sequence identity to SEQ ID NO: 19,
e) a cellulase having at least 60%, at least 65%, at least 70%, at least 75%, at least 80%, at least 85%, at least 90%, at least 95%, at least 98% or 100% sequence identity to SEQ ID NO: 37, and
f) a cellulase having at least 60%, at least 65%, at least 70%, at least 75%, at least 80%, at least 85%, at least 90%, at least 95%, at least 98% or 100% sequence identity to SEQ ID NO: 38.

One embodiment relates to a cleaning composition comprising a beta-glucanase, a DNase, an additional cellulase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the cellulase has at least 60%, at least 65%, at least 70%, at least 75%, at least 80%, at least 85%, at least 90%, at least 95%, at least 98% or 100% sequence identity to SEQ ID NO: 16, and is preferably obtained from Bacillus.

One embodiment relates to a cleaning composition comprising a beta-glucanase, a DNase, an additional cellulase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the cellulase has at least 60%, at least 65%, at least 70%, at least 75%, at least 80%, at least 85%, at least 90%, at least 95%, at least 98% or 100% sequence identity to SEQ ID NO; 17, and is preferably obtained from Humicola e.g. Humicola insolens.

One embodiment relates to a cleaning composition comprising a beta-glucanase, a DNase, an additional cellulase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the cellulase has at least 60%, at least 65%, at least 70%, at least 75%, at least 80%, at least 85%, at least 90%, at least 95%, at least 98% or 100% sequence identity to SEQ ID NO: 18, and is preferably obtained from Humicola e.g. Humicola insolens.

One embodiment relates to a cleaning composition comprising a beta-glucanase, a DNase, an additional cellulase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the cellulase has at least 60%, at least 65%, at least 70%, at least 75%, at least 80%, at least 85%, at least 90%, at least 95%, at least 98% or 100% sequence identity to SEQ ID NO: 19, and is preferably obtained from Thielavia e.g. Thielavia terrestris.

One embodiment relates to a cleaning composition comprising a beta-glucanase, a DNase, an additional cellulase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the cellulase has at least 60%, at least 65%, at least 70%, at least 75%, at least 80%, at least 85%, at least 90%, at least 95%, at least 98% or 100% sequence identity to SEQ ID NO: 37, and is preferably obtained from Paenibacillus e.g. Paenibacillus polymyxa.

One embodiment relates to a cleaning composition comprising a beta-glucanase, a DNase, an additional cellulase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the cellulase has at least 60%, at least 65%, at least 70%, at least 75%, at least 80%, at least 85%, at least 90%, at least 95%, at least 98% or 100% sequence identity to SEQ ID NO: 38, and is preferably obtained from Melanocarpus e.g. Melanocarpus albomyces

One preferred cleaning enzyme to be combined with the beta-glucanse enzyme and the DNase enzyme is a lipase. Lipases are enzymes that catalyzes the hydrolysis of fats (lipids). Lipases are used in detergents for removal of grease stains. Lipases E.C. 3.1.1. are a subclass of the esterases E.C. 3.1. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcafigenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

One embodiment relates to a cleaning composition comprising a beta-glucanase, a DNase, a lipase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the lipase belongs to E.C. 3.1.1.

One embodiment relates to a cleaning composition comprising a beta-glucanase, a DNase, a lipase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the lipase is obtained from Thermomyces, e.g. from T. lanuginosus, Pseudomonas e.g. P. alcaligenes or P. pseudoalcaligenes, P. cepacia, P. sp. strain SD705, P. wisconsinensis, Pseudomonas mendocina, Streptomyces, Magnaporthe e.g. M. grisea, Thermobifida e.g. T. fusca, Geobacillus e.g. G. stearothermophilus, Bacillus e.g. B. subtilis, Streptomyces e.g. S. griseus or S. pristinaespiralis.

One embodiment relates to a cleaning composition comprising a beta-glucanase, a DNase, an lipase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the lipase is a lipase having at least 60%, at least 65%, at least 70%, at least 75%, at least 80%, at least 85%, at least 90%, at least 95%, at least 98% or 100% sequence identity to SEQ ID NO: 20, or a lipase having at least 60%, at least 65%, at least 70%, at least 75%, at least 80%, at least 85%, at least 90%, at least 95%, at least 98% but less than 100% sequence identity to SEQ ID NO: 20 comprising one or more of the substitutions selected from the group consisting of D27R, G38A, G91A/Q, D96E, G163K, T231R, N233R, D254S and P256T, compared to SEQ ID NO: 20, wherein each position corresponds to the position in SEQ ID NO: 20.

One embodiment relates to a cleaning composition comprising a beta-glucanase, a DNase, an lipase, and a cleaning component, wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the lipase is a lipase having at least 60%, at least 65%, at least 70%, at least 75%, at least 80%, at least 85%, at least 90%, at least 95%, at least 98% but less than 100% sequence identity to SEQ ID NO: 20, wherein the lipase comprises one or both the substitutions T231R and/or N233R, wherein the position corresponds to the positions of SEQ ID NO: 20.

One preferred cleaning enzyme to be combined with the beta-glucanase and the DNase enzyme is a mannanase, Mannanases are enzyme catalyzing hydrolyses of 1,4-beta-D-mannosidic linkages in mannans, galactomannans, glucomannans, and galactoglucomannans. Mannans are a type of hemicellulose representing up to 25% of wood dry weight in softwoods, but are also found in other plant material, especially in a variety of seeds. Mannans are polysaccharides with a backbone of β-1,4-linked D-mannopyranosyl residues, which can contain galactose or acetyl substitutions and may have glucose residues in the backbone. The main enzyme type participating in the degradation of mannans are endo-1,4-β-mannanases (EC 3.2.1.78), which hydrolyze the internal glycoside bonds in the mannan backbone. The present invention provides a cleaning composition comprising a beta-glucanase, a DNase and a mannanase enzyme comprising a polypeptide having mannan endo-1,4-beta-mannosidase activity (EC 3.2.1.78) that catalyzes the hydrolysis of 1,4-β-D-mannosidic linkages in mannans, galactomannans and/or glucomannans. According to CAZy (www.cazy.org), endo-1,4-β-mannanases can be found in glycoside hydrolyase families 5, 26 and 113. Couturier et al. have reported a GH26 mannanase from Podospora anserina having 56.1% and 76.4% identity to SEQ ID NO: 3 and 6 respectively in (2013), “Structural and Biochemical Analyses of Glycoside Hydrolase Families 5 and 26-(1,4)-Mannanases from Podospora anserina Reveal Differences upon Manno-oligosaccharide Catalysis”, J. Biol. Chem., 288(20): 14624-14635. Preferred mannanases include the GH5 mannanase obtained from Bacillus bogoriensis described in WO1999/064619 or any of the GH26 Mannanases, mannanase from Preussia aemulans WO 2017/021515 (SEQ ID NO 2), mannanase from Yunnania penicillata WO2017/021516 (SEQ ID NO 2), mannanase from Myrothecium roridum WO2017/021517 (SEQ ID NO 2), mannanase from Chaetomium brasiliense WO2017/021518 (SEQ ID NO 2), mannanases from Ascobolus stictoideus or mannanase from Chaetomium virescens SEQ ID NO 3 and 6 from WO2015/040159.

One embodiment relates to a cleaning composition comprising a beta-glucanase, a DNase, an mannanase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the mannanase belongs to EC 3.2.1.78.

One embodiment relates to a cleaning composition comprising a beta-glucanase, a DNase, an mannanase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the mannanase is a GH5 or a GH26 mannanase.

One embodiment relates to a cleaning composition comprising a beta-glucanase, a DNase, an mannanase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the mannanase is obtained from Bacillus e.g. B. bogoriensis or hemicellulosilyticus, Paenibacillus e.g. P. woosongensis or P. illinoisensis, Neobulgaria sp., Preussia e.g. P. aemulans, Yunnania e.g. Y. penicillata, Myrothecium e.g. M. roridum, Chaetomium e.g. C. brasiliense, Ascobolus e.g. A. stictoideus or Chaetomium e.g. C. virescens.

One embodiment relates to a cleaning composition comprising a beta-glucanase, a DNase, an mannanase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the mannanase is selected from the group consisting of;
a) a mannanase, wherein the mannanase preferably belongs to the Glycoside Hydrolase Family 5 mannanases;
   i. a mannanase having at least 60%, at least 65%, at least 70%, at least 75%, at least 80%, at least 85%, at least 90%, at least 95%, at least 98% or 100% sequence identity to SEQ ID NO: 21;
b) a mannanase wherein the mannanase preferably belongs to the Glycoside Hydrolase Family 26 mannanases;
   i. a mannanase having at least 60%, at least 65%, at least 70%, at least 75%, at least 80%, at least 85%, at least 90%, at least 95%, at least 98% or 100% sequence identity to SEQ ID NO: 22;
   ii. a mannanase having at least 60%, at least 65%, at least 70%, at least 75%, at least 80%, at least 85%, at least 90%, at least 95%, at least 98% or 100% sequence identity to SEQ ID NO: 23;
   iii. a mannanase having at least 60%, at least 65%, at least 70%, at least 75%, at least 80%, at least 85%, at least 90%, at least 95%, at least 98% or 100% sequence identity to SEQ ID NO: 24;
   iv. a mannanase having at least 60%, at least 65%, at least 70%, at least 75%, at least 80%, at least 85%, at least 90%, at least 95%, at least 98% or 100% sequence identity to SEQ ID NO: 25;
   v. a mannanase having at least 60%, at least 65%, at least 70%, at least 75%, at least 80%, at least 85%, at least 90%, at least 95%, at least 98% or 100% sequence identity to SEQ ID NO: 26;
   vi. a mannanase having at least 60%, at least 65%, at least 70%, at least 75%, at least 80%, at least 85%, at least 90%, at least 95%, at least 98% or 100% sequence identity to SEQ ID NO: 27;
   vii. a mannanase having at least 60%, at least 65%, at least 70%, at least 75%, at least 80%, at least 85%, at least 90%, at least 95%, at least 98% or 100% sequence identity to SEQ ID NO: 28;
   viii. a mannanase having at least 60%, at least 65%, at least 70%, at least 75%, at least 80%, at least 85%, at least 90%, at least 95%, at least 98% or 100% sequence identity to SEQ ID NO: 29;
   ix. a mannanase having at least 60%, at least 65%, at least 70%, at least 75%, at least 80%, at least 85%, at least 90%, at least 95%, at least 98% or 100% sequence identity to SEQ ID NO: 30;
   x. a mannanase having at least 60%, at least 65%, at least 70%, at least 75%, at least 80%, at least 85%, at least 90%, at least 95%, at least 98% or 100% sequence identity to SEQ ID NO: 39; and
   xi. a mannanase having at least 60%, at least 65%, at least 70%, at least 75%, at least 80%, at least 85%, at least 90%, at least 95%, at least 98% or 100% sequence identity to SEQ ID NO: 40.

One embodiment relates to a cleaning composition comprising a beta-glucanase, a DNase, an mannanase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the mannanase has at least 60%, at least 65%, at least 70%, at least 75%, at least 80%, at least 85%, at least 90%, at least 95%, at least 98% or 100% sequence identity to SEQ ID NO: 21, and preferably is obtained from Bacillus e.g. Bacillus bogoriensis.

One embodiment relates to a cleaning composition comprising a beta-glucanase, a DNase, an mannanase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the mannanase has at least 60%, at least 65%, at least 70%, at least 75%, at least 80%, at least 85%, at least 90%, at least 95%, at least 98% or 100% sequence identity to SEQ ID NO: 22, and preferably is obtained from Paenibacillus e.g. Paenibacillus woosongensis

One embodiment relates to a cleaning composition comprising a beta-glucanase, a DNase, an mannanase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the mannanase has at least 60%, at least 65%, at least 70%, at least 75%, at least 80%, at least 85%, at least 90%, at least 95%, at least 98% or 100% sequence identity to SEQ ID NO: 23, and preferably is obtained from Paenibacillus e.g. Paenibacillus illinoisensis.

One embodiment relates to a cleaning composition comprising a beta-glucanase, a DNase, an mannanase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the mannanase has at least 60%, at least 65%, at least 70%, at least 75%, at least 80%, at least 85%, at least 90%, at least 95%, at least 98% or 100% sequence identity to SEQ ID NO: 24, and preferably is obtained from Neobulgaria sp.

One embodiment relates to a cleaning composition comprising a beta-glucanase, a DNase, an mannanase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the mannanase has at least 60%, at least 65%, at least 70%, at least 75%, at least 80%, at least 85%, at least 90%, at least 95%, at least 98% or 100% sequence identity to SEQ ID NO: 25, and preferably is obtained from Preussia e.g. Preussia aemulans.

One embodiment relates to a cleaning composition comprising a beta-glucanase, a DNase, an mannanase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the mannanase has at least 60%, at least 65%, at least 70%, at least 75%, at least 80%, at least 85%, at least 90%, at least 95%, at least 98% or 100% sequence identity to SEQ ID NO: 26, and preferably is obtained from Yunnania e.g. Yunnania penicillate.

One embodiment relates to a cleaning composition comprising a beta-glucanase, a DNase, an mannanase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the mannanase has at least 60%, at least 65%, at least 70%, at least 75%, at least 80%, at least 85%, at least 90%, at least 95%, at least 98% or 100% sequence identity to SEQ ID NO: 27, and preferably is obtained from Myrothecium e.g. Myrothecium roridum.

One embodiment relates to a cleaning composition comprising a beta-glucanase, a DNase, an mannanase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the mannanase has at least 60%, at least 65%, at least 70%, at least 75%, at least 80%, at least 85%, at least 90%, at least 95%, at least 98% or 100% sequence identity to SEQ ID NO: 28, and preferably is obtained from Chaetomium e.g. Chaetomium brasiliense.

One embodiment relates to a cleaning composition comprising a beta-glucanase, a DNase, an mannanase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the mannanase has at least 60%, at least 65%, at least 70%, at least 75%, at least 80%, at least 85%, at least 90%, at least 95%, at least 98% or 100% sequence identity to SEQ ID NO: 29, and preferably is obtained from Ascobolus e.g. Ascobolus stictoideus.

One embodiment relates to a cleaning composition comprising a beta-glucanase, a DNase, an mannanase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the mannanase has at least 60%, at least 65%, at least 70%, at least 75%, at least 80%, at least 85%, at least 90%, at least 95%, at least 98% or 100% sequence identity to SEQ ID NO: 30, and preferably is obtained from Chaetomium e.g. Chaetomium virescens.

One embodiment relates to a cleaning composition comprising a beta-glucanase, a DNase, an mannanase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the mannanase has at least 60%, at least 65%, at least 70%, at least 75%, at least 80%, at least 85%, at least 90%, at least 95%, at least 98% or 100% sequence identity to SEQ ID NO: 39, and preferably is obtained from Paenibacillus.

One embodiment relates to a cleaning composition comprising a beta-glucanase, a DNase, an mannanase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the mannanase has at least 60%, at least 65%, at least 70%, at least 75%, at least 80%, at least 85%, at least 90%, at least 95%, at least 98% or 100% sequence identity to SEQ ID NO: 40, and preferably is obtained from Bacillus e.g. Bacillus hemicellulosilyticus.

One preferred cleaning enzyme to be combined with the beta-glucanase and the DNase enzyme is a protease. Proteases are enzymes that hydrolyze peptide bonds. The most frequently used protease for household care segment is the serine proteases (or serine endopeptidases), E.C. 3.4.21, which are enzymes that cleave peptide bonds in proteins, in which serine serves as the nucleophilic amino acid at the active site. Suitable proteases include those of bacterial, fungal, plant, viral or animal origin e.g. vegetable or microbial origin. Microbial origin is preferred. Preferably the protease is a subtilase and even more preferably the protease belongs to the subtilisin sub group of subtilases. Most of the proteases used in the cleaning industry today are obtained from Bacillus e.g. Bacillus lentus and Bacillus amyloliquefaciens. One embodiment of the invention relates to a cleaning composition comprising a DNase, a protease and at least one cleaning component, wherein the protease is obtained from Bacillus preferably from Bacillus lentus, Bacillus amyloliquefaciens, Bacillus gibsonii, Bacillus licheniformis, Bacillus pumilus, Bacillus halodurans or Bacillus subtilis.

One embodiment relates to a cleaning composition comprising a beta-glucanase, a DNase, a protease, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the protease belongs to E.C. 3.4.21. One embodiment relates to a cleaning composition comprising a beta-glucanase, a DNase, a protease, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the protease is obtained from Bacillus preferably from Bacillus lentus, Bacillus amyloliquefaciens, Bacillus licheniformis, Bacillus pumilus, Bacillus halodurans or Bacillus subtilis.

One embodiment relates to a cleaning composition comprising a beta-glucanase, a DNase, a protease, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the protease is selected from the group consisting of;
a) a protease having at least 60%, at least 65%, at least 70%, at least 75%, at least 80%, at least 85%, at least 90%, at least 95%, at least 98%, at least 99% or 100% sequence identity to SEQ ID NO: 31, preferably obtained from Bacillus lentus;
b) a protease having at least 60%, at least 65%, at least 70%, at least 75%, at least 80%, at least 85%, at least 90%, at least 95%, at least 98%, at least 99% or 100% sequence identity to SEQ ID NO: 32; preferably obtained from Bacillus amyloliquefaciens;
c) a protease having at least 60%, at least 65%, at least 70%, at least 75%, at least 80%, at least 85%, at least 90%, at least 95%, at least 98%, at least 99% or 100% sequence identity to SEQ ID NO: 33, preferably obtained from Bacillus sp.;
d) a protease having at least 60%, at least 65%, at least 70%, at least 75%, at least 80%, at least 85%, at least 90%, at least 95%, at least 98%, at least 99% or 100% sequence identity to SEQ ID NO: 34, preferably obtained from Bacillus gibsonii;
e) a protease having at least 60%, at least 65%, at least 70%, at least 75%, at least 80%, at least 85%, at least 90%, at least 95%, at least 98%, at least 99% or 100% sequence identity to SEQ ID NO: 35, preferably obtained from Bacillus lentus;
f) a protease having at least 60%, at least 65%, at least 70%, at least 75%, at least 80%, at least 85%, at least 90%, at least 95%, at least 98%, at least 99% or 100% sequence identity to SEQ ID NO: 36, preferably obtained from Bacillus licheniformis;
g) or a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises the substitution T22R or T22A compared to SEQ ID NO: 31, wherein the position corresponds to the position of SEQ ID NO: 31;
h) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises one or more, preferably all the substitutions selected from the group consisting of: S3T, V41, A188P and V199I, compared to SEQ ID NO: 31, wherein the positions correspond to the positions of SEQ ID NO: 31;
i) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises one or more, preferably all the substitutions selected from the group consisting of N114L, T207A, A226V, and E265F, compared to SEQ ID NO: 31, wherein the positions correspond to the positions of SEQ ID NO: 31;
j) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises one or more, preferably all substitutions selected from the group consisting of: S97D, S101A, V102I and G157S compared to SEQ ID NO: 31, wherein the positions correspond to the positions of SEQ ID NO: 31;
k) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises one or more, preferably all the substitutions selected from the group consisting of: S85N, G116V, S126L, P127Q and S128A compared to SEQ ID NO: 31, wherein the positions correspond to the positions of SEQ ID NO: 31;
l) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises one or more, preferably all the substitutions selected from the group consisting of: Y161A, R164S and A188P, compared to SEQ ID NO: 31, wherein the positions correspond to the positions of SEQ ID NO: 31;
m) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises one or more, preferably all the substitutions selected from the group consisting of: S3T, R19L, and A188P, wherein the positions correspond to the positions of SEQ ID NO: 31;
n) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises one or more, preferably all the substitutions selected from the group consisting of: S9R, R19L, and N60D, wherein the positions correspond to the positions of SEQ ID NO: 31;
o) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises the amino acid Arginine (R), at a position corresponding to a position selected from the group consisting of: 9, 42 and 239 of SEQ ID NO: 31;
p) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises the amino acid Glutamic acid (E) or Aspartic acid (D), at a position corresponding to a position selected from the group consisting of: 9, 42, 60, 61, 74, 157, 176, 179, 182, 212, 250, 253 and 256 of SEQ ID NO: 31;
q) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises an insertion of the amino acid Aspartic acid (D) or Glutamic acid (E) at a position corresponding to position 97 of SEQ ID NO: 31;
r) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises the amino acid selected from the group consisting of: Glutamic acid (E), Aspartic acid (D), Glycine (G), Arginine (R) and Methionine (M) at a position corresponding to position 99 of SEQ ID NO: 31;
s) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises the amino acid selected from the group consisting of: Glutamic acid (E), Aspartic acid (D) and Glutamine (Q), at a position corresponding to position 211 of SEQ ID NO: 31;
t) a protease having at least 60%, at least 65%, at least 70%, at least 75%, at least 80%, at least 85%, at least 90%, at least 95%, at least 98% but less than 100% sequence identity to SEQ ID NO: 32, wherein the protease comprises the amino acid selected from the group consisting of: Glutamic acid (E), Aspartic acid (D) and Glutamine (Q), at a position corresponding to position 217 of SEQ ID NO: 32;
u) a protease having at least 60%, at least 65%, at least 70%, at least 75%, at least 80%, at least 85%, at least 90%, at least 95%, at least 98% but less than 100% sequence identity to SEQ ID NO: 32, wherein the protease comprises one or more of the substitutions selected from the group consisting of: S24G/R, S53G, S78N, S101N, G128A/S and Y217Q/L, compared to SEQ ID NO: 32, wherein the positions correspond to the positions of SEQ ID NO: 32.

One preferred cleaning enzyme to be combined with the beta-glucanase and the DNase enzyme is a pectinase. Pectinases are a group of enzymes that cleave glycosidic linkages of pectic substances mainly poly(1,4-alpha-D-galacturonide and its derivatives (see reference Sakai et al., Pectin, pectinase and protopectinase: production, properties and applications, pp 213-294 in: Advances in Applied Microbiology vol:39,1993). Preferably the pectinase catalyzes the random cleavage of alpha-1,4-glycosidic linkages in pectic acid also called polygalacturonic acid by transelimination such as the enzyme class polygalacturonate lyase (EC 4.2.2.2) (PGL) also known as poly(1,4-alpha-D-galacturonide) lyase also known as pectate lyase.

One embodiment relates to a cleaning composition comprising a beta-glucanase, a DNase, a pectinase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the pectinase is obtained from Bacillus preferably from Bacillus subtilis.

One embodiment relates to a cleaning composition comprising a beta-glucanase, a DNase, a pectinase, and a cleaning component, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence shown in SEQ ID NO: 1, SEQ ID NO: 2, SEQ ID NO: 3, SEQ ID NO: 4, or SEQ ID NO: 5, and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 6 or SEQ ID NO: 7, wherein the pectinase has at least 60%, at least 65%, at least 70%, at least 75%, at least 80%, at least 85%, at least 90%, at least 95%, at least 98%, at least 99% or 100% sequence identity to SEQ ID NO: 42, preferably obtained from Bacillus subtilis.

### Additional Enzymes

### Enzymes

The cleaning composition may comprise one or more additional enzymes such as one or more pectinase, arabinase, galactanase, xylanase, oxidase, e.g., a laccase, and/or peroxidase. In general, the properties of the selected enzyme(s) should be compatible with the selected detergent, (i.e., pH-optimum, compatibility with other enzymatic and non-enzymatic ingredients, etc.), and the enzyme(s) should be present in effective amounts.

### Peroxidases/Oxidases

Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include Guardzyme™ (Novozymes A/S). A peroxidase is comprised by the enzyme classification EC 1.11.1.7, as set out by the Nomenclature Committee of the International Union of Biochemistry and Molecular Biology (IUBMB), or any fragment derived therefrom, exhibiting peroxidase activity. Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257. A suitable peroxidase includes a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions. Preferably, the haloperoxidase is a vanadium haloperoxidase, i.e., a vanadate-containing haloperoxidase. Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis. Haloperoxidases have also been isolated from bacteria such as Pseudomonas, e.g., P. pyrrocinia and Streptomyces, e.g., S. aureofaciens. A suitable oxidase includes in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5). Preferred laccase enzymes are enzymes of microbial origin. The enzymes may be derived from plants, bacteria or fungi (including filamentous fungi and yeasts). Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885). Suitable examples from bacteria include a laccase derivable from a strain of Bacillus. A laccase derived from Coprinopsis or Myceliophthora is preferred; in particular, a laccase derived from Coprinopsis cinerea, as disclosed in WO 97/08325; or from Myceliophthora thermophila, as disclosed in WO 95/33836.

The enzymes may be included in the cleaning composition of the present invention at a level of from 0.01 to 1000 ppm, from 1 ppm to 1000 ppm, from 10 ppm to 1000 ppm, from 50 ppm to 1000 ppm, from 100 ppm to 1000 ppm, from 150 ppm to 1000 ppm, from 200 ppm to 1000 ppm, from 250 ppm to 1000 ppm, from 250 ppm to 750 ppm, from 250 ppm to 500 ppm.

The DNases above may be combined with enzymes to form a blend to be added to the wash liquor solution according to the invention. The concentration of the beta-glucanase in the wash liquor solution is typically in the range of wash liquor from 0.00001 ppm to 10 ppm, from 0.00002 ppm to 10 ppm, from 0.0001 ppm to 10 ppm, from 0.0002 ppm to 10 ppm, from 0.001 ppm to 10 ppm, from 0.002 ppm to 10 ppm, from 0.01 ppm to 10 ppm, from 0.02 ppm to 10 ppm, 0.1 ppm to 10 ppm, from 0.2 ppm to 10 ppm, from 0.5 ppm to 5 ppm. The concentration of the DNase in the wash liquor solution is typically in the range of wash liquor from 0.00001 ppm to 10 ppm, from 0.00002 ppm to 10 ppm, from 0.0001 ppm to 10 ppm, from 0.0002 ppm to 10 ppm, from 0.001 ppm to 10 ppm, from 0.002 ppm to 10 ppm, from 0.01 ppm to 10 ppm, from 0.02 ppm to 10 ppm, 0.1 ppm to 10 ppm, from 0.2 ppm to 10 ppm, from 0.5 ppm to 5 ppm. The concentration of the additional enzyme i.e. the amylase, cellulase, lipase, mannanase or the protease in the wash liquor solution is typically in the range of wash liquor from 0.00001 ppm to 10 ppm, from 0.00002 ppm to 10 ppm, from 0.0001 ppm to 10 ppm, from 0.0002 ppm to 10 ppm, from 0.001 ppm to 10 ppm, from 0.002 ppm to 10 ppm, from 0.01 ppm to 10 ppm, from 0.02 ppm to 10 ppm, 0.1 ppm to 10 ppm, from 0.2 ppm to 10 ppm, from 0.5 ppm to 5 ppm.
The DNases may be combined with any of the enzymes of the invention to form a blend to be added to a composition according to the invention.

One embodiment relates to a cleaning composition comprising a DNase, enzymes of the invention and at least one cleaning component, wherein the amount of beta-glucanase is from 0.01 to 1000 ppm, the amount of DNase in the composition is from 0.01 to 1000 ppm and the amount of protease is from 0.01 to 1000 ppm.

In addition to the amylase, cellulase, lipase, mannanase or protease, the beta-glucanase, and the DNase the cleaning composition further comprises one or more cleaning component. One embodiment relates to a cleaning composition comprising a beta-glucanase, a DNase, an amylase, cellulase, lipase, mannanase or protease and at least one cleaning component, wherein the cleaning component is selected from surfactants, preferably anionic and/or nonionic, builders and bleach components.

The choice of cleaning components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

### Surfactants

The cleaning composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 0.1% to about 15% or about 3% to about 10%. The surfactant(s) is chosen based on the desired cleaning application, and may include any conventional surfactant(s) known in the art.

When included therein the detergent will usually contain from about 1% to about 40% by weight of an anionic surfactant, such as from about 5% to about 30%, including from about 5% to about 15%, or from about 15% to about 20%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

When included therein the detergent will usually contain from about 1% to about 40% by weigh of a cationic surfactant, for example from about 0.5% to about 30%, in particular from about 1% to about 20%, from about 3% to about 10%, such as from about 3% to about 5%, from about 8% to about 12% or from about 10% to about 12%. Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

When included therein the detergent will usually contain from about 0.2% to about 40% by weight of a nonionic surfactant, for example from about 0.5% to about 30%, in particular from about 1% to about 20%, from about 3% to about 10%, such as from about 3% to about 5%, from about 8% to about 12%, or from about 10% to about 12%. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

When included therein the detergent will usually contain from about 0.01 to about 10% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, and combinations thereof.

When included therein the detergent will usually contain from about 0.01% to about 10% by weight of a zwitterionic surfactant. Non-limiting examples of zwitterionic surfactants include betaines such as alkyldimethylbetaines, sulfobetaines, and combinations thereof.

### Builders and Co-Builders

The cleaning composition may contain about 0-65% by weight, such as about 5% to about 50%, such as about 0.5% to about 20% of a detergent builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg. Any builder and/or co-builder known in the art for use in cleaning detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The detergent composition may also contain 0-50% by weight, such as about 5% to about 30%, of a detergent co-builder. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N, N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SM DA), N-(2-hydroxyethyl)ethylenediamine-N,N′,N″-triacetic acid (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053

### Bleaching Systems

The cleaning composition may contain 0-30% by weight, such as about 1% to about 20%, such as about 0.01% to about 10% of a bleaching system. Any bleaching system comprising components known in the art for use in cleaning detergents may be utilized. Suitable bleaching system components include sources of hydrogen peroxide; sources of peracids; and bleach catalysts or boosters.

Sources of Hydrogen Peroxide:

Suitable sources of hydrogen peroxide are inorganic persalts, including alkali metal salts such as sodium percarbonate and sodium perborates (usually mono- or tetrahydrate), and hydrogen peroxide—urea (1/1).

Sources of Peracids:

Peracids may be (a) incorporated directly as preformed peracids or (b) formed in situ in the wash liquor from hydrogen peroxide and a bleach activator (perhydrolysis) or (c) formed in situ in the wash liquor from hydrogen peroxide and a perhydrolase and a suitable substrate for the latter, e.g., an ester.
a) Suitable preformed peracids include, but are not limited to, peroxycarboxylic acids such as peroxybenzoic acid and its ring-substituted derivatives, peroxy-α-naphthoic acid, peroxyphthalic acid, peroxylauric acid, peroxystearic acid, ε-phthalimidoperoxycaproic acid [phthalimidoperoxyhexanoic acid (PAP)], and o-carboxybenzamidoperoxycaproic acid; aliphatic and aromatic diperoxydicarboxylic acids such as diperoxydodecanedioic acid, diperoxyazelaic acid, diperoxysebacic acid, diperoxybrassylic acid, 2-decyldiperoxybutanedioic acid, and diperoxyphthalic, -isophthalic and -terephthalic acids; perimidic acids; peroxymonosulfuric acid; peroxydisulfuric acid; peroxyphosphoric acid; peroxysilicic acid; and mixtures of said compounds. It is understood that the peracids mentioned may in some cases be best added as suitable salts, such as alkali metal salts (e.g., Oxone®) or alkaline earth-metal salts.

b) Suitable bleach activators include those belonging to the class of esters, amides, imides, nitriles or anhydrides and, where applicable, salts thereof. Suitable examples are tetraacetylethylenediamine (TAED), sodium 4-[(3,5,5-trimethylhexanoyl)oxy]benzene-1-sulfonate (ISONOBS), sodium 4-(dodecanoyloxy)benzene-1-sulfonate (LOBS), sodium 4-(decanoyloxy)benzene-1-sulfonate, 4-(decanoyloxy)benzoic acid (DOBA), sodium 4-(nonanoyloxy)benzene-1-sulfonate (NOBS), and/or those disclosed in WO98/17767. A particular family of bleach activators of interest is disclosed in EP624154 and particularly preferred in that family is acetyl triethyl citrate (ATC). ATC or a short chain triglyceride like triacetin has the advantage that they are environmentally friendly. Furthermore, acetyl triethyl citrate and triacetin have good hydrolytical stability in the product upon storage and are efficient bleach activators. Finally, ATC is multifunctional, as the citrate released in the perhydrolysis reaction may function as a builder.

### Bleach Catalysts and Boosters

The bleaching system may also include a bleach catalyst or booster.

Some non-limiting examples of bleach catalysts that may be used in the compositions of the present invention include manganese oxalate, manganese acetate, manganese-collagen, cobalt-amine catalysts and manganese triazacyclononane (MnTACN) catalysts; particularly preferred are complexes of manganese with 1,4,7-trimethyl-1,4,7-triazacyclononane (Me3-TACN) or 1,2,4,7-tetramethyl-1,4,7-triazacyclononane (Me4-TACN), in particular Me3-TACN, such as the dinuclear manganese complex [(Me3-TACN)Mn(O)3Mn(Me3-TACN)](PF6)2, and [2,2′,2″-nitrilotris(ethane-1,2-diylazanylylidene-KN-methanylylidene)triphenolato-κ3O]manganese(III). The bleach catalysts may also be other metal compounds; such as iron or cobalt complexes.

In some embodiments, where a source of a peracid is included, an organic bleach catalyst or bleach booster may be used having one of the following formulae:

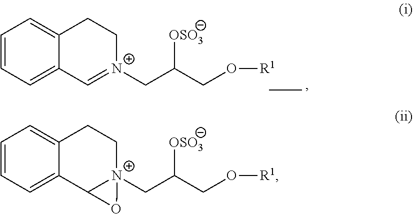

(iii) and mixtures thereof; wherein each R1 is independently a branched alkyl group containing from 9 to 24 carbons or linear alkyl group containing from 11 to 24 carbons, preferably each R1 is independently a branched alkyl group containing from 9 to 18 carbons or linear alkyl group containing from 11 to 18 carbons, more preferably each R1 is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl.
Other exemplary bleaching systems are described, e.g. in WO2007/087258, WO2007/087244, WO2007/087259, EP1867708 (Vitamin K) and WO2007/087242. Suitable photobleaches may for example be sulfonated zinc or aluminium phthabcyanines.

### Metal Care Agents

Metal care agents may prevent or reduce the tarnishing, corrosion or oxidation of metals, including aluminium, stainless steel and non-ferrous metals, such as silver and copper. Suitable examples include one or more of the following:
(a) benzatriazoles, including benzotriazole or bis-benzotriazole and substituted derivatives thereof. Benzotriazole derivatives are those compounds in which the available substitution sites on the aromatic ring are partially or completely substituted. Suitable substituents include linear or branch-chain C1-C20-alkyl groups (e.g., C1-C20-alkyl groups) and hydroxyl, thio, phenyl or halogen such as fluorine, chlorine, bromine and iodine.
(b) metal salts and complexes chosen from the group consisting of zinc, manganese, titanium, zirconium, hafnium, vanadium, cobalt, gallium and cerium salts and/or complexes, the metals being in one of the oxidation states II, III, IV, V or VI. In one aspect, suitable metal salts and/or metal complexes may be chosen from the group consisting of Mn(II) sulphate, Mn(II) citrate, Mn(II) stearate, Mn(II) acetylacetonate, K\hat{ }TiF6 (e.g., K2TiF6), K\hat{ }ZrF6 (e.g., K2ZrF6), CoSO4, Co(NOs)2 and Ce(NOs)3, zinc salts, for example zinc sulphate, hydrozincite or zinc acetate.; (c) silicates, including sodium or potassium silicate, sodium disilicate, sodium metasilicate, crystalline phyllosilicate and mixtures thereof.
Further suitable organic and inorganic redox-active substances that act as silver/copper corrosion inhibitors are disclosed in WO 94/26860 and WO 94/26859. Preferably the composition of the invention comprises from 0.1 to 5% by weight of the composition of a metal care agent, preferably the metal care agent is a zinc salt.

### Hydrotropes

The cleaning composition may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Polymers

The cleaning composition may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Suitable examples include PVP-K15, PVP-K30, ChromaBond S-400, ChromaBond S-403E and Chromabond S-100 from Ashland Aqualon, and Sokalan® HP 165, Sokalan® HP 50 (Dispersing agent), Sokalan® HP 53 (Dispersing agent), Sokalan® HP 59 (Dispersing agent), Sokalan® HP 56 (dye transfer inhibitor), Sokalan® HP 66 K (dye transfer inhibitor) from BASF. Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated. Particularly preferred polymer is ethoxylated homopolymer Sokalan® HP 20 from BASF, which helps to prevent redeposition of soil in the wash liquor.

### Fabric Hueing Agents

The cleaning compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

### Dispersants

The cleaning compositions of the present invention can also contain dispersants. In particular, powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

### Dye Transfer Inhibiting Agents

The cleaning compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

### Fluorescent Whitening Agent

The cleaning compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2,2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins. Suitable fluorescent brightener levels include lower levels of from about 0.01, from 0.05, from about 0.1 or even from about 0.2 wt % to upper levels of 0.5 or even 0.75 wt %.

### Soil Release Polymers

The cleaning compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers is amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore, random graft co-polymers are suitable soil release polymers. Suitable graft co-polymers are described in more detail in WO 2007/138054, WO 2006/108856 and WO 2006/113314 (hereby incorporated by reference). Suitable polyethylene glycol polymers include random graft co-polymers comprising: (i) hydrophilic backbone comprising polyethylene glycol; and (ii) side chain(s) selected from the group consisting of: C4-C25 alkyl group, polypropylene, polybutylene, vinyl ester of a saturated C1-C6 mono-carboxylic acid, CI-C 6 alkyl ester of acrylic or methacrylic acid, and mixtures thereof. Suitable polyethylene glycol polymers have a polyethylene glycol backbone with random grafted polyvinyl acetate side chains. The average molecular weight of the polyethylene glycol backbone can be in the range of from 2,000 Da to 20,000 Da, or from 4,000 Da to 8,000 Da. The molecular weight ratio of the polyethylene glycol backbone to the polyvinyl acetate side chains can be in the range of from 1:1 to 1:5, or from 1:1.2 to 1:2. The average number of graft sites per ethylene oxide units can be less than 1, or less than 0.8, the average number of graft sites per ethylene oxide units can be in the range of from 0.5 to 0.9, or the average number of graft sites per ethylene oxide units can be in the range of from 0.1 to 0.5, or from 0.2 to 0.4. A suitable polyethylene glycol polymer is Sokalan HP22. Other soil release polymers are substituted polysaccharide structures especially substituted cellulosic structures such as modified cellulose derivatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

### Anti-Redeposition Agents

The cleaning compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

### Rheology Modifiers

The cleaning compositions of the present invention may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.
Other suitable cleaning composition components include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Formulation of Detergent Products

The cleaning compositions of the present invention may be formulated, for example, as a hand or machine laundry detergent composition including a laundry additive composition suitable for pre-treatment of stained fabrics and a rinse added fabric softener composition or be formulated as a detergent composition for use in general household hard surface cleaning operations or be formulated for hand or machine dishwashing operations. In a specific aspect, the present invention provides a detergent additive comprising one or more enzymes as described herein. The cleaning composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water-soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water-soluble film. The compartment for liquid components can be different in composition than compartments containing solids: US2009/0011970 A1. Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent. A liquid or gel detergent may be non-aqueous.

### Granular Cleaning Formulations

Non-dusting granulates may be produced, e.g. as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

The beta-glucanase and the DNase may be formulated as a granule for example as a co-granule that combines one or more enzymes. Each enzyme will then be present in more granules securing a more uniform distribution of enzymes in the detergent. This also reduces the physical segregation of different enzymes due to different particle sizes. Methods for producing multi-enzyme co-granulate for the detergent industry is disclosed in the IP.com disclosure IPCOM000200739D.

Another example of formulation of enzymes by the use of co-granulates are disclosed in WO 2013/188331, which relates to a detergent composition comprising (a) a multi-enzyme co-granule; (b) less than 10 wt zeolite (anhydrous basis); and (c) less than 10 wt phosphate salt (anhydrous basis), wherein said enzyme co-granule comprises from 10 to 98 wt % moisture sink component and the composition additionally comprises from 20 to 80 wt % detergent moisture sink component. The multi-enzyme co-granule may comprise a beta-glucanase and a DNase and an amylase, cellulase, lipase, mannanase or protease and one or more enzymes selected from the group consisting of xyloglucanases, perhydrolases, peroxidases, lipoxygenases, laccases, hemicellulases, cellobiose dehydrogenases, xylanases, esterases, cutinases, pectinases, mannanases, pectate lyases, keratinases, reductases, oxidases, phenoloxidases, ligninases, pullulanases, tannases, pentosanases, lichenases glucanases, arabinosidases, hyaluronidase, chondroitinase and mixtures thereof. WO 2013/188331 also relates to a method of treating and/or cleaning a surface, preferably a fabric surface comprising the steps of (i) contacting said surface with the detergent composition as claimed and described herein in aqueous wash liquor, (ii) rinsing and/or drying the surface.

An embodiment of the invention relates to an enzyme granule/particle comprising the beta-glucanase, the DNase, and an amylase, cellulase, lipase, mannanase or protease or combination thereof. The granule is composed of a core, and optionally one or more coatings (outer layers) surrounding the core. Typically, the granule/particle size, measured as equivalent spherical diameter (volume based average particle size), of the granule is 20-2000 μm, particularly 50-1500 μm, 100-1500 μm or 250-1200 μm. The core may include additional materials such as fillers, fibre materials (cellulose or synthetic fibres), stabilizing agents, solubilising agents, suspension agents, viscosity regulating agents, light spheres, plasticizers, salts, lubricants and fragrances. The core may include binders, such as synthetic polymer, wax, fat, or carbohydrate. The core may comprise a salt of a multivalent cation, a reducing agent, an antioxidant, a peroxide decomposing catalyst and/or an acidic buffer component, typically as a homogenous blend. The core may consist of an inert particle with the enzyme absorbed into it, or applied onto the surface, e.g., by fluid bed coating. The core may have a diameter of 20-2000 μm, particularly 50-1500 μm, 100-1500 μm or 250-1200 μm. The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in Handbook of Powder Technology; Particle size enlargement by C. E. Capes; Volume 1; 1980; Elsevier.

The core of the enzyme granule/particle may be surrounded by at least one coating, e.g., to improve the storage stability, to reduce dust formation during handling, or for coloring the granule. The optional coating(s) may include a salt coating, or other suitable coating materials, such as polyethylene glycol (PEG), methyl hydroxy-propyl cellulose (MHPC) and polyvinyl alcohol (PVA). Examples of enzyme granules with multiple coatings are shown in WO 93/07263 and WO 97/23606. The coating may be applied in an amount of at least 0.1% by weight of the core, e.g., at least 0.5%, 1% or 5%. The amount may be at most 100%, 70%, 50%, 40% or 30%. The coating is preferably at least 0.1 μm thick, particularly at least 0.5 μm, at least 1 μm or at least 5 μm. In a one embodiment, the thickness of the coating is below 100 μm. In another embodiment, the thickness of the coating is below 60 μm. In an even more particular embodiment the total thickness of the coating is below 40 μm. The coating should encapsulate the core unit by forming a substantially continuous layer. A substantially continuous layer is to be understood as a coating having few or no holes, so that the core unit it is encapsulating/enclosing has few or none uncoated areas. The layer or coating should be homogeneous in thickness. The coating can further contain other materials as known in the art, e.g., fillers, antisticking agents, pigments, dyes, plasticizers and/or binders, such as titanium dioxide, kaolin, calcium carbonate or talc. A salt coating may comprise at least 60% by weight w/w of a salt, e.g., at least 65%, at least 70%, at least 75%, at least 80%, at least 85%, at least 90%, at least 95% or at least 99% by weight w/w. The salt may be added from a salt solution where the salt is completely dissolved or from a salt suspension wherein the fine particles is less than 50 μm, such as less than 10 μm or less than 5 μm. The salt coating may comprise a single salt or a mixture of two or more salts. The salt may be water soluble and may have a solubility at least 0.1 grams in 100 g of water at 20° C., preferably at least 0.5 g per 100 g water, e.g., at least 1 g per 100 g water, e.g., at least 5 g per 100 g water. The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminium. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, lactate, formate, acetate, butyrate, propionate, benzoate, tartrate, ascorbate or gluconate. In particular alkali- or earth alkali metal salts of sulfate, sulfite, phosphate, phosphonate, nitrate, chloride or carbonate or salts of simple organic acids such as citrate, malonate or acetate may be used. The salt in the coating may have a constant humidity at 20° C. above 60%, particularly above 70%, above 80% or above 85%, or it may be another hydrate form of such a salt (e.g., anhydrate). The salt coating may be as described in WO 00/01793 or WO 2006/034710. Specific examples of suitable salts are NaCl (CH_{20° C.}=76%), Na₂CO₃ (CH_{20° C.}=92%), NaNO₃ (CH_{20° C.}=73%), Na₂HPO₄ (CH_{20° C.}=95%), Na₃PO₄ (CH_{25° C.}=92%), NH₄Cl (CH_{20° C.}=79.5%), (NH₄)₂HPO₄ (CH_{20° C.}=93.0%), NH₄H₂PO₄ (CH_{20° C.}=93.1%), (NH₄)₂SO₄ (CH_{20° C.}=81.1%), KCl (CH_{20° C.}=85%), K₂HPO₄ (CH_{20° C.}=92%), KH₂PO₄ (CH_{20° C.}=96.5%), KNO₃ (CH_{20° C.}=93.5%), Na₂SO₄ (CH_{20° C.}=93%), K₂SO₄ (CH_{20° C.}=98%), KHSO₄ (CH_{20° C.}=86%), MgSO₄ (CH_{20° C.}=90%), ZnSO₄ (CH_{20° C.}=90%) and sodium citrate (CH_{25° C.}=86%). Other examples include NaH₂PO₄, (NH₄)H₂PO₄, CuSO₄, Mg(NO₃)₂ and magnesium acetate. The salt may be in anhydrous form, or it may be a hydrated salt, i.e. a crystalline salt hydrate with bound water(s) of crystallization, such as described in WO 99/32595. Specific examples include anhydrous sodium sulfate (Na₂SO₄), anhydrous magnesium sulfate (MgSO₄), magnesium sulfate heptahydrate (MgSO₄.7H₂O), zinc sulfate heptahydrate (ZnSO₄.7H₂O), sodium phosphate dibasic heptahydrate (Na₂HPO₄.7H₂O), magnesium nitrate hexahydrate (Mg(NO₃)₂(6H₂O)), sodium citrate dihydrate and magnesium acetate tetrahydrate. Preferably the salt is applied as a solution of the salt, e.g., using a fluid bed.

### Uses

The present invention is also directed to methods for using the compositions thereof. Laundry/textile/fabric (House hold laundry washing, Industrial laundry washing). Hard surface cleaning (ADW, car wash, Industrial surface). The cleaning e.g. detergent composition of the present invention may be formulated, for example, as a hand or machine laundry detergent composition including a laundry additive composition suitable for pre-treatment of stained fabrics and a rinse added fabric softener composition or be formulated as a detergent composition for use in general household hard surface cleaning operations or be formulated for hand or machine dishwashing operations. In a specific aspect, the present invention provides a detergent additive comprising one or more enzymes as described herein.

The compositions of the invention comprise a blend of beta-glucanase, DNase and amylase, cellulase, lipase, mannanase or protease and effectively reduce or remove organic components, such as starch, grease, protein and DNA stains from surfaces such as textiles and hard surfaces e.g. dishes.

One embodiment of the invention relates to the use of a composition comprising a beta-glucanase, a DNase and an amylase, cellulase, lipase, mannanase or protease for reduction of redeposition. One embodiment of the invention relates to the use of a cleaning composition comprising a beta-glucanase, a DNase and an amylase, cellulase, lipase, mannanase or protease for reduction of redeposition.

One embodiment of the invention relates to the use of a cleaning composition comprising a beta-glucanase, a DNase and an amylase, cellulase, lipase, mannanase or protease for reduction of redeposition when the cleaning composition is applied in e.g. laundry process. One embodiment of the invention relates to the use of a cleaning composition comprising a beta-glucanase, a DNase and an amylase, cellulase, lipase, mannanase or protease for reduction of redeposition on an item e.g. textile. In one embodiment, the composition is an anti-redeposition composition.

### EXAMPLES

### Assays

### Assay I: Determination of Beta-Glucanase (Licheninase or Lichenase) Activity:

An AZCL-Barley beta-glucan (azurine dye covalently cross-linked beta-glucan) assay was used for detection of endo-glucancase activity (Licheninase or Lichenase activity).
AZCL-Barley beta-glucan (75 mg) was suspended in 15 mL detergent (Model detergents A, X, Z with and without bleach and pH adjusted, ADW Model A). To 1 mL of this solution in Eppendorf tubes was added 10 μL enzyme (0.33 mg enzyme protein/Liter), incubated for 15 min at 40° C. while shaking at 1250 rpm in a pre-heated thermo mixer and spun down for 2 min at 13200 rpm, diluted 5 times with a 5% Triton-X-100 including 10 μM CaCl2 and 250 μL of the solution was transferred to a micro-titer plate and the sample absorbance was measured at 590 nm.

### Assay II: Testing of DNase Activity

DNase activity is determined on DNase Test Agar with Methyl Green (BD, Franklin Lakes, N.J., USA), which is prepared according to the manual from supplier. Briefly, 21 g of agar is dissolved in 500 ml water and then autoclaved for 15 min at 121° C. Autoclaved agar is temperated to 48° C. in water bath, and 20 ml of agar is poured into petri dishes with and allowed to solidify by incubation o/n at room temperature. On solidified agar plates, 5 μl of enzyme solutions are added and DNase activity is observed as colorless zones around the spotted enzyme solutions

### Assay III: Testing of DNase Activity

DNase activity is determined by using the DNaseAlert™ Kit (11-02-01-04, IDT Intergrated DNA Technologies) according to the supplier's manual. Briefly, 95 μl DNase sample is mixed with 5 μl substrate in a microtiter plate, and fluorescence is immediately measured using a Clariostar microtiter reader from BMG Labtech (536 nm excitation, 556 nm emission).

### Assay IV: Testing of Alpha-Amylase Activity

The alpha-amylase activity may be determined by a method employing the G7-pNP substrate. G7-pNP which is an abbreviation for 4,6-ethylidene(G₇)-p-nitrophenyl(G₁)-α,D-maltoheptaoside, a blocked oligosaccharide which can be cleaved by an endo-amylase, such as an alpha-amylase. Following the cleavage, the alpha-Glucosidase included in the kit digest the hydrolysed substrate further to liberate a free PNP molecule which has a yellow color and thus can be measured by visible spectophometry at λ=405 nm (400-420 nm.). Kits containing G7-pNP substrate and alpha-Glucosidase is manufactured by Roche/Hitachi (cat. No. 11876473). The G7-pNP substrate from this kit contains 22 mM 4,6-ethylidene-G7-pNP and 52.4 mM HEPES (2-[4-(2-hydroxyethyl)-1-piperazinyl]-ethanesulfonic acid), pH 7.0). The alpha-Glucosidase reagent contains 52.4 mM HEPES, 87 mM NaCl, 12.6 mM MgCl₂, 0.075 mM CaCl₂, >4 kU/L alpha-glucosidase). The substrate working solution is made by mixing 1 mL of the alpha-Glucosidase reagent with 0.2 mL of the G7-pNP substrate. This substrate working solution is made immediately before use. Dilution buffer: 50 mM MOPS, 0.05% (w/v) Triton X100 (polyethylene glycol p-(1,1,3,3-tetramethylbutyl)-phenyl ether (C₁₄H₂₂O(C₂H₄O)ₙ (n=9-10))), 1 mM CaCl₂), pH8.0. The amylase sample to be analyzed is diluted in dilution buffer to ensure the pH in the diluted sample is 7. The assay is performed by transferring 20 μl diluted enzyme samples to 96 well microtiter plate and adding 80 μl substrate working solution. The solution is mixed and pre-incubated 1 minute at room temperature and absorption is measured every 20 sec. over 5 minutes at OD 405 nm. The slope (absorbance per minute) of the time dependent absorption-curve is directly proportional to the specific activity (activity per mg enzyme) of the alpha-amylase in question under the given set of conditions. The amylase sample should be diluted to a level where the slope is below 0.4 absorbance units per minute.

### Assay V: Testing of Cellulase Activity

An AZCL-He-cellulose (azurine dye covalently cross-linked cellulose) assay is used for detection of cellulase (endo-glucanase) activity. AZCL-He-cellulose (75 mg) is suspended in 15 mL detergent (e.g. Model detergent A). To 1 mL of this solution in Eppendorf tubes is added 100 μL enzyme (0.09 mg enzyme protein/mL), incubated for 15 min at 40° C. while shaking at 1250 rpm in a pre-heated thermo mixer and spun down for 2 min at 13200 rpm. 250 μL of the solution is transferred to a micro-titer plate and the sample absorbance is measured at 590 nm.

### Assay VI: Testing of Lipase Activity

Lipase is diluted with a buffer (10 mM Succinic acid+2 mM CaCl₂)+0.02% Brij 35 adjusted to pH6.5) to the specified concentration. 10 uL of the 100 ppm lipase solution is added to a 90 uL of detergent composition, stirred for 5 minutes and sealed. Samples are stored at 4° C. in detergent D002 (unstressed) and in detergent D002 at 47° C. (stressed). Storage time is 335.5 hours. After storage possible condensation liquid is collected by centrifugation. To the 100 uL stressed or unstressed sample 235 uL of buffer (0.1M Tris-HCl, 9 mM CaCl₂), 0.0225% Brij-30, pH8.0+0.85% 4-FBPA (31.5 g/l)) are added corresponding to a 3.35-fold dilution. After 10 minutes stirring 5 uL sample aliquots are further diluted with the same buffer 60-fold. Then one part of this lipase dilution is mixed with four parts of 0.5 mM pNP-palmitate, 1 mM calcium chloride, 100 mM Tris (pH8.0), 6.5 mM Deoxycholate, 1.4 g/L AOS and for 30 minutes release of the pNP chromophore is measured spectrophotometrically. This is used to determine activity via the initial linear slope of the reaction. Residual activity is calculated as the ratio of the measured velocities of stressed versus unstressed sample. The median value of the residual activity is calculated based on four replicates and normalized by a lipase variant reference run with each experimental set.

### Assay VII: Testing of Mannanase Activity

Mannanase activity may be tested according to standard test procedures known in the art, such as by applying a solution to be tested to 4 mm diameter holes punched out in agar plates containing 0.2% AZCL galactomannan (carob), i.e. substrate for the assay of endo-1,4-beta-D-mannanase available as CatNo.I-AZGMA from the company Megazyme (Megazyme's Internet address: http://www.megazyme.com/Purchase/index.html).

### Assay VIII: Testing of Protease Activity

Proteolytic activity can be determined by a method employing Suc-AAPF-PNA as the substrate. Suc-AAPF-PNA is an abbreviation for N-Succinyl-Alanine-Alanine-Proline-Phenylalanine-p-Nitroanilide and is a blocked peptide which can be cleaved by endo-proteases. Following cleavage, a free PNA molecule is liberated, which has a yellow color and thus can be measured by visible spectrophotometry at wavelength 405 nm. The Suc-AAPF-PNA substrate is manufactured by Bachem (cat. no. L1400, dissolved in DMSO). The protease sample to be analyzed is diluted in residual activity buffer (100 mM Tris pH 8.6). The assay is performed by transferring 3 0 μl of diluted enzyme samples to 96 well microtiter plate and adding 70 μl substrate working solution (0.72 mg/ml in 100 mM Tris pH8.6). The solution is mixed at room temperature and absorption is measured every 20 seconds over 5 minutes at OD 405 nm. The slope (absorbance per minute) of the time dependent absorption-curve is directly proportional to the activity of the protease in question under the given set of conditions. The protease sample is diluted to a level where the slope is linear.

### Detergent Compositions

The below mentioned detergent composition can be used in combination with the enzyme combinations of the invention.

### Biotex Black (Liquid)

5-15% Anionic surfactants, <5% Nonionic surfactants, perfume, enzymes, DMDM and hydantoin.

### Composition of Ariel Sensitive White & Color, Liquid Detergent Composition

Aqua, Alcohol Ethoxy Sulfate, Alcohol Ethoxylate, Amino Oxide, Citrid Acid, C12-18 topped palm kernel fatty acid, Protease, Glycosidase, Amylase, Ethanol, 1,2 Propanediol, Sodium Formate, Calcium Chloride, Sodium hydroxide, Silicone Emulsion, Trans-sulphated EHDQ (the ingredients are listed in descending order).

### Composition of WFK IEC-A Model Detergent (Powder)

Ingredients: Linear sodium alkyl benzene sulfonate 8,8%, Ethoxylated fatty alcohol C12-18 (7 EO) 4.7%, Sodium soap 3.2%, Anti foam DC2-4248S 3.9%, Sodium aluminium silicate zeolite 4A 28.3%, Sodium carbonate 11.6%, Sodium salt of a copolymer from acrylic and maleic acid (Sokalan CP5) 2.4%, Sodium silicate 3.0%, Carboxymethylcellulose 1.2%, Dequest 2066 2.8%, Optical whitener 0.2%, Sodium sulfate 6.5%, Protease 0.4%.

### Composition of Model Detergent A (Liquid)

Ingredients: 12% LAS, 11% AEO Biosoft N25-7 (NI), 7% AEOS (SLES), 6% MPG (monopropylene glycol), 3% ethanol, 3% TEA, 2.75% cocoa soap, 2.75% soya soap, 2% glycerol, 2% sodium hydroxide, 2% sodium citrate, 1% sodium formiate, 0.2% DTMPA and 0.2% PCA (all percentages are w/w)

### Composition of Ariel Actilift (Liquid)

Ingredients: 5-15% Anionic surfactants; <5% Non-ionic surfactants, Phosphonates, Soap; Enzymes, Optical brighteners, Benzisothiazolinone, Methylisothiazolinone, Perfumes, Alpha-isomethyl ionone, Citronellol, Geraniol, Linalool.

### Composition of Ariel Actilift Colour & Style (Liquid)

Ingredients: 5-15% Anionic surfactants; <5% Non-ionic surfactants, Phosphonates, Soap; Enzymes, Perfumes, Benzisothiazolinone, Methylisothiazolinone, Alpha-isomethyl ionone, Butylphenyl methylpropional, Citronellol, Geraniol, Linalool.

### Composition of Persil Small & Mighty (Liquid)

Ingredients: 15-30% Anionic surfactants, Non-ionic surfacts, 5-15% Soap, <5% Polycarboxylates, Perfume, Phosphates, Optical Brighteners

Persil 2 Int with Comfort Passion Flower Powder

Sodium sulfate, Sodium carbonate, Sodium Dodecylbenzenesulfonate, Bentonite, Sodium Carbonate Peroxide, Sodium Silicate, Zeolite, Aqua, Citric acid, TAED, C12-15 Pareth-7, Stearic Acid, Parfum, Sodium Acrylic Acid/MA Copolymer, Cellulose Gum, Corn Starch Modified, Sodium chloride, Tetrasodium Etidronate, Calcium Sodium EDTMP, Disodium Anilinomorpholinotriazinyl-aminostilbenesulfonate, Sodium bicarbonate, Phenylpropyl Ethyl Methicone, Butylphenyl Methylpropional, Glyceryl Stearates, Calcium carbonate, Sodium Polyacrylate, Alpha-Isomethyl Ionone, Disodium Distyrylbiphenyl Disulfonate, Cellulose, Protease, Limonene, PEG-75, Titanium dioxide, Dextrin, Sucrose, Sodium Polyaryl Sulphonate, CI 12490, CI 45100, CI 42090, Sodium Thiosulfate, CI 61585.

### Persil Biological Powder

Sucrose, Sorbitol, Aluminum Silicate, Polyoxymethylene Melamine, Sodium Polyaryl Sulphonate, CI 61585, CI 45100, Lipase, Amylase, Xanthan gum, Hydroxypropyl methyl cellulose, CI 12490, Disodium Distyrylbiphenyl Disulfonate, Sodium Thiosulfate, CI 42090, Mannanase, CI 11680, Etidronic Acid, Tetrasodium EDTA.

### Persil Biological Tablets

Sodium carbonate, Sodium Carbonate Peroxide, Sodium bicarbonate, Zeolite, Aqua, Sodium Silicate, Sodium Lauryl Sulfate, Cellulose, TAED, Sodium Dodecylbenzenesulfonate, Hemicellulose, Lignin, Lauryl Glucoside, Sodium Acrylic Acid/MA Copolymer, Bentonite, Sodium chloride, Parfum, Tetrasodium Etidronate, Sodium sulfate, Sodium Polyacrylate, Dimethicone, Disodium Anilinomorpholinotriazinylaminostilbenesulfonate, Dodecylbenzene Sulfonic Acid, Trimethylsiloxysilicate, Calcium carbonate, Cellulose, PEG-75, Titanium dioxide, Dextrin, Protease, Corn Starch Modified, Sucrose, CI 12490, Sodium Polyaryl Sulphonate, Sodium Thiosulfate, Amylase, Kaolin,

### Persil Colour Care Biological Powder

Subtilisin, Imidazolidinone, Hexyl Cinnamal, Sucrose, Sorbitol, Aluminum Silicate, Polyoxymethylene Melamine, CI 61585, CI 45100, Lipase, Amylase, Xanthan gum, Hydroxypropyl methyl cellulose, CI 12490, Disodium Distyrylbiphenyl Disulfonate, Sodium Thiosulfate, CI 42090, Mannanase, CI 11680, Etidronic Acid, Tetrasodium EDTA.

### Persil Colour Care Biological Tablets

Sodium bicarbonate, Sodium carbonate, Zeolite, Aqua, Sodium Silicate, Sodium Lauryl Sulfate, Cellulose Gum, Sodium Dodecylbenzenesulfonate, Lauryl Glucoside, Sodium chloride, Sodium Acrylic Acid/MA Copolymer, Parfum, Sodium Thioglycolate, PVP, Sodium sulfate, Tetrasodium Etidronate, Sodium Polyacrylate, Dimethicone, Bentonite, Dodecylbenzene Sulfonic Acid, Trimethylsiloxysilicate, Calcium carbonate, Cellulose, PEG-75, Titanium dioxide, Dextrin, Protease, Corn Starch Modified, Sucrose, Sodium Thiosulfate, Amylase, CI 74160, Kaolin.

### Persil Dual Action Capsules Bio

MEA-Dodecylbenzenesulfonate, MEA-Hydrogenated Cocoate, C12-15 Pareth-7, Dipropylene Glycol, Aqua, Tetrasodium Etidronate, Polyvinyl Alcohol, Glycerin, Aziridine, homopolymer ethoxylated, Propylene glycol, Parfum, Sodium Diethylenetriamine Pentamethylene Phosphonate, Sorbitol, MEA-Sulfate, Ethanolamine, Subtilisin, Glycol, Butylphenyl Methylpropional, Boronic acid, (4-formylphenyl), Hexyl Cinnamal, Limonene, Linalool, Disodium Distyrylbiphenyl Disulfonate, Alpha-Isomethyl Ionone, Geraniol, Amylase, Polymeric Blue Colourant, Polymeric Yellow Colourant, Talc, Sodium chloride, Benzisothiazolinone, Mannanase, Denatonium Benzoate.

Persil 2 int with Comfort Sunshiny Days Powder

Sodium sulfate, Sodium carbonate, Sodium Dodecylbenzenesulfonate, Bentonite, Sodium Carbonate Peroxide, Sodium Silicate, Zeolite, Aqua, Citric acid, TAED, C12-15 Pareth-7, Parfum, Stearic Acid, Sodium Acrylic Acid/MA Copolymer, Cellulose Gum, Corn Starch Modified, Sodium chloride, Tetrasodium Etidronate, Calcium Sodium EDTMP, Disodium Anilinomorpholinotriazinyl-aminostilbenesulfonate, Sodium bicarbonate, Phenylpropyl Ethyl Methicone, Butylphenyl Methylpropional, Glyceryl Stearates, Calcium carbonate, Sodium Polyacrylate, Geraniol, Disodium Distyrylbiphenyl Disulfonate, Cellulose, Protease, PEG-75, Titanium dioxide, Dextrin, Sucrose, Sodium Polyaryl Sulphonate, CI 12490, CI 45100, CI 42090, Sodium Thiosulfate, CI 61585.

Persil Small & Mighty 2 in1 with Comfort Sunshiny Days

Aqua, C12-15 Pareth-7, Sodium Dodecylbenzenesulfonate, Propylene glycol, Sodium Hydrogenated Cocoate, Triethanolamine, Glycerin, TEA-Hydrogenated Cocoate, Parfum, Sodium chloride, Polyquaternium-10, PVP, Polymeric Pink Colourant, Sodium sulfate, Disodium Distyrylbiphenyl Disulfonate, Butylphenyl Methylpropional, Styrene/Acrylates Copolymer, Hexyl Cinnamal, Citronellol, Eugenol, Polyvinyl Alcohol, Sodium acetate, Isopropyl alcohol, Polymeric Yellow Colourant, Sodium Lauryl Sulfate.

### Persil Small & Mighty Bio

Aqua, MEA-Dodecylbenzenesulfonate, Propylene glycol, Sodium Laureth Sulfate, C12-15 Pareth-7, TEA-Hydrogenated Cocoate, MEA-Citrate, Aziridine homopolymer ethoxylated, MEA-Etidronate, Triethanolamine, Parfum, Acrylates Copolymer, Sorbitol, MEA-Sulfate, Sodium Sulfite, Disodium Distyrylbiphenyl Disulfonate, Butylphenyl Methylpropional, Styrene/Acrylates Copolymer, Citronellol, Sodium sulfate, Peptides, salts, sugars from fermentation (process), Subtilisin, Glycerin, Boronic acid, (4-formylphenyl), Geraniol, Pectate Lyase, Amylase, Sodium Lauryl Sulfate, Mannanase, CI 42051.

### Persil Small & Mighty Capsules Biological

MEA-Dodecylbenzenesulfonate, MEA-Hydrogenated Cocoate, C12-15 Pareth-7, Dipropylene Glycol, Aqua, Glycerin, Polyvinyl Alcohol, Parfum, Aziridine homopolymer ethoxylated, Sodium Diethylenetriamine Pentamethylene Phosphonate, Propylene glycol, Sorbitol, MEA-Sulfate, Ethanolamine, Subtilisin, Glycol, Butylphenyl Methylpropional, Hexyl Cinnamal, Starch, Boronic acid, (4-formylphenyl), Limonene, Linalool, Disodium Distyrylbiphenyl Disulfonate, Alpha-Isomethyl Ionone, Geraniol, Amylase, Talc, Polymeric Blue Colourant, Sodium chloride, Benzisothiazolinone, Denatonium Benzoate, Polymeric Yellow Colourant, Mannanase.

### Persil Small & Mighty Capsules Colour Care

MEA-Dodecylbenzenesulfonate, MEA-Hydrogenated Cocoate, C12-15 Pareth-7, Dipropylene Glycol, Aqua, Glycerin, Polyvinyl Alcohol, Parfum, Aziridine homopolymer ethoxylated, Sodium Diethylenetriamine Pentamethylene Phosphonate, Propylene glycol, MEA-Sulfate, Ethanolamine, PVP, Sorbitol, Butylphenyl Methylpropional, Subtilisin, Hexyl Cinnamal, Starch, Limonene, Linalool, Boronic acid, (4-formylphenyl), Alpha-Isomethyl Ionone, Geraniol, Talc, Polymeric Blue Colourant, Denatonium Benzoate, Polymeric Yellow Colourant.

### Persil Small & Mighty Colour Care

Aqua, MEA-Dodecylbenzenesulfonate, Propylene glycol, Sodium Laureth Sulfate, C12-15 Pareth-7, TEA-Hydrogenated Cocoate, MEA-Citrate, Aziridine homopolymer ethoxylated, MEA-Etidronate, Triethanolamine, Parfum, Acrylates Copolymer, Sorbitol, MEA-Sulfate, Sodium Sulfite, Glycerin, Butylphenyl Methylpropional, Citronellol, Sodium sulfate, Peptides, salts, sugars from fermentation (process), Styrene/Acrylates Copolymer, Subtilisin, Boronic acid, (4-formylphenyl), Geraniol, Pectate Lyase, Amylase, Sodium Lauryl Sulfate, Mannanase, CI 61585, CI 45100.

Composition of Fairy Non Bio (liquid)

Ingredients: 15-30% Anionic Surfactants, 5-15% Non-Ionic Surfactants, Soap, Benzisothiazolinone, Methylisothiazolinone, Perfumes

### Composition of Model Detergent T (Powder)

Ingredients: 11% LAS, 2% AS/AEOS, 2% soap, 3% AEO, 15.15% sodium carbonate, 3% sodium silicate, 18.75% zeolite, 0.15% chelant, 2% sodium citrate, 1.65% AA/MA copolymer, 2.5% CMC and 0.5% SRP (all percentages are w/w).

### Composition of Model Detergent X (Powder)

Ingredients: 16.5% LAS, 15% zeolite, 12% sodium disilicate, 20% sodium carbonate, 1% sokalan, 35.5% sodium sulfate (all percentages are w/w).

### Composition of Ariel Actilift Colour & Style (Powder)

Ingredients: 15-30% Anionic surfactants, <5% Non-ionic surfactants, Phosphonates, Polycarboxylates, Zeolites; Enzymes, Perfumes, Hexyl cinnamal.

### Composition of Ariel Actilift (Powder)

Ingredients: 5-15% Anionic surfactants, Oxygen-based bleaching agents, <5% Non-ionic surfactants, Phosphonates, Polycarboxylates, Zeolites, Optical brightners, Enzymes, Perfumes, Butylphenyl Methylpropional, Coumarin, Hexyl Cinnamal

### Composition of Persil Megaperls (Powder)

Ingredients: 15-30% of the following: anionic surfactants, oxygen-based bleaching agent and zeolites, less than 5% of the following: non-ionic surfactants, phosphonates, polycarboxylates, soap, Further ingredients: Perfumes, Hexyl cinnamal, Benzyl salicylate, Linalool, optical brighteners, Enzymes and Citronellol.

### Gain Liquid, Original:

Ingredients: Water, Alcohol Ethoxysulfate, Diethylene Glycol, Alcohol Ethoxylate, Ethanolamine, Linear Alkyl Benzene Sulfonate, Sodium Fatty Acids, Polyethyleneimine Ethoxylate, Citric Acid, Borax, Sodium Cumene Sulfonate, Propylene Glycol, DTPA, Disodium

Diaminostilbene Disulfonate, Dipropylethyl Tetramine, Sodium Hydroxide, Sodium Formate, Calcium Formate, Dimethicone, Amylase, Protease, Liquitint™, Hydrogenated Castor Oil, Fragrance

### Tide Liquid, Original:

Ingredients: Linear alkylbenzene sulfonate, propylene glycol, citric acid, sodium hydroxide, borax, ethanolamine, ethanol, alcohol sulfate, polyethyleneimine ethoxylate, sodium fatty acids, diquaternium ethoxysulfate, protease, diethylene glycol, laureth-9, alkyldimethylamine oxide, fragrance, amylase, disodium diaminostilbene disulfonate, DTPA, sodium formate, calcium formate, polyethylene glycol 4000, mannanase, Liquitint™ Blue, dimethicone.

### Liquid Tide, Free and Gentle:

Water, sodium alcoholethoxy sulfate, propylene glycol, borax, ethanol, linear alkylbenzene sulfonate sodium, salt, polyethyleneimine ethoxylate, diethylene glycol, trans sulfated & ethoxylated hexamethylene diamine, alcohol ethoxylate, linear alkylbenzene sulfonate, MEA salt, sodium formate, sodium alkyl sulfate, DTPA, amine oxide, calcium formate, disodium diaminostilbene, disulfonate, amylase, protease, dimethicone, benzisothiazolinone

### Tide Coldwater Liquid, Fresh Scent:

Water, alcoholethoxy sulfate, linear alkylbenzene sulfonate, diethylene glycol, propylene glycol, ethanolamine, citric acid, Borax, alcohol sulfate, sodium hydroxide, polyethyleneimine, ethoxylate, sodium fatty acids, ethanol, protease, Laureth-9, diquaternium ethoxysulfate, lauramine oxide, sodium cumene, sulfonate, fragrance, DTPA, amylase, disodium, diaminostilbene, disulfonate, sodium formate, disodium distyrylbiphenyl disulfonate, calcium formate, polyethylene glycol 4000, mannanase, pectinase, Liquitint™ Blue, dimethicone

### Tide TOTALCARE™ Liquid, Cool Cotton:

Water, alcoholethoxy sulfate, propylene glycol, sodium fatty acids, laurtrimonium chloride, ethanol, sodium hydroxide, sodium cumene sulfonate, citric acid, ethanolamine, diethylene glycol, silicone polyether, borax, fragrance, polyethyleneimine ethoxylate, protease, Laureth-9,

DTPA, polyacrylamide quaternium chloride, disodium diaminostilbene disulfonate, sodium formate, Liquitint™ Orange, dipropylethyl tetraamine, dimethicone, cellulase,

### Liquid Tide Plus Bleach Alternative™, Vivid White and Bright, Original and Clean Breeze:

Water, sodium alcoholethoxy sulfate, sodium alkyl sulfate, MEA citrate, linear alkylbenzene sulfonate, MEA salt, propylene glycol, diethylene glycol, polyethyleneimine ethoxylate, ethanol, sodium fatty acids, ethanolamine, lauramine oxide, borax, Laureth-9, DTPA, sodium cumene sulfonate, sodium formate, calcium formate, linear alkylbenzene sulfonate, sodium salt, alcohol sulfate, sodium hydroxide, diquaternium ethoxysulfate, fragrance, amylase, protease, mannanase, pectinase, disodium diaminostilbene disulfonate, benzisothiazolinone, Liquitint™ Blue, dimethicone, dipropylethyl tetraamine.

### Liquid Tide HE, Original Scent:

Water, Sodium alcoholethoxy sulfate, MEA citrate, Sodium Alkyl Sulfate, alcohol ethoxylate, linear alkylbenzene sulfonate, MEA salt, sodium fatty acids, polyethyleneimine ethoxylate, diethylene glycol, propylene glycol, diquaternium ethoxysulfate, borax, polyethyleneimine, ethoxylate propoxylate, ethanol, sodium cumene sulfonate, fragrance, DTPA, disodium diaminostilbene disulfonate, Mannanase, cellulase, amylase, sodium formate, calcium formate, Lauramine oxide, Liquitint™ Blue, Dimethicone/polydimethyl silicone.

### Tide TOTALCARE HE Liquid, Renewing Rain:

Water, alcoholethoxy sulfate, linear alkylbenzene sulfonate, alcohol ethoxylate, citric acid, Ethanolamine, sodium fatty acids, diethylene glycol, propylene glycol, sodium hydroxide, borax, polyethyleneimine ethoxylate, silicone polyether, ethanol, protease, sodium cumene sulfonate, diquaternium ethoxysulfate, Laureth-9, fragrance, amylase, DTPA, disodium diaminostilbene disulfonate, disodium distyrylbiphenyl disulfonate, sodium formate, calcium formate, mannanase, Liquitint™ Orange, dimethicone, polyacrylamide quaternium chloride, cellulase, dipropylethyl tetraamine.

Tide liquid HE Free:

Water, alcoholethoxy sulfate, diethylene glycol, monoethanolamine citrate, sodium formate, propylene glycol, linear alkylbenzene sulfonates, ethanolamine, ethanol, polyethyleneimine ethoxylate, amylase, benzisothiazolin, borax, calcium formate, citric acid, diethylenetriamine pentaacetate sodium, dimethicone, diquaternium ethoxysulfate, disodium diaminostilbene disulfonate, Laureth-9, mannanase, protease, sodium cumene sulfonate, sodium fatty acids.

### Tide Coldwater HE Liquid, Fresh Scent:

Water, alcoholethoxy sulfate, MEA Citrate, alcohol sulfate, Alcohol ethoxylate, Linear alkylbenzene sulfonate MEA, sodium fatty acids, polyethyleneimine ethoxylate, diethylene glycol, propylene glycol, diquaternium ethoxysulfate, borax, polyethyleneimine ethoxylate propoxylate, ethanol, sodium cumene sulfonate, fragrance, DTPA, disodium diaminostilbene disulfonate, protease, mannanase, cellulase, amylase, sodium formate, calcium formate, lauramine oxide, Liquitint™ Blue, dimethicone.

### Tide for Coldwater HE Free Liquid:

Water, sodium alcoholethoxy sulfate, MEA Citrate, Linear alkylbenzene sulfonate: sodium salt, Alcohol ethoxylate, Linear alkylbenzene sulfonate: MEA salt, sodium fatty acids, polyethyleneimine ethoxylate, diethylene glycol, propylene glycol, diquaternium ethoxysulfate, Borax, protease, polyethyleneimine ethoxylate propoxylate, ethanol, sodium cumene sulfonate, Amylase, citric acid, DTPA, disodium diaminostilbene disulfonate, sodium formate, calcium formate, dimethicone.

### Tide Simply Clean & Fresh:

Water, alcohol ethoxylate sulfate, linear alkylbenzene sulfonate Sodium/Mea salts, propylene glycol, diethylene glycol, sodium formate, ethanol, borax, sodium fatty acids, fragrance, lauramine oxide, DTPA, Polyethylene amine ethoxylate, calcium formate, disodium diaminostilbene disulfonate, dimethicone, tetramine, Liquitint™ Blue.

### Tide Pods, Ocean Mist, Mystic Forest, Spring Meadow:

Linear alkylbenzene sulfonates, C12-16 Pareth-9, propylene glycol, alcoholethoxy sulfate, water, polyethyleneimine ethoxylate, glycerine, fatty acid salts, PEG-136 polyvinyl acetate, ethylene Diamine disuccinic salt, monoethanolamine citrate, sodium bisulfite, diethylenetriamine pentaacetate sodium, disodium distyrylbiphenyl disulfonate, calcium formate, mannanase, exyloglucanase, sodium formate, hydrogenated castor oil, natalase, dyes, termamyl, subtilisin, benzisothiazolin, perfume.

### Tide to Go:

Deionized water, Dipropylene Glycol Butyl Ether, Sodium Alkyl Sulfate, Hydrogen Peroxide, Ethanol, Magnesium Sulfate, Alkyl Dimethyl Amine Oxide, Citric Acid, Sodium Hydroxide, Trimethoxy Benzoic Acid, Fragrance.

### Tide Stain Release Liquid:

Water, Alkyl Ethoxylate, Linear Alkylbenzenesulfonate, Hydrogen Peroxide, Diquaternium Ethoxysulfate, Ethanolamine, Disodium Distyrylbiphenyl Disulfonate, tetrabutyl Ethylidinebisphenol, F&DC Yellow 3, Fragrance.

### Tide Stain Release Powder:

Sodium percarbonate, sodium sulfate, sodium carbonate, sodium aluminosilicate, nonanoyloxy benzene sulfonate, sodium polyacrylate, water, sodium alkylbenzenesulfonate, DTPA, polyethylene glycol, sodium palmitate, amylase, protease, modified starch, FD&C Blue 1, fragrance.

### Tide Stain Release, Pre Treater Spray:

Water, Alkyl Ethoxylate, MEA Borate, Linear Alkylbenzenesulfonate, Propylene Glycol, Diquaternium Ethoxysulfate, Calcium Chlorideenzyme, Protease, Ethanolamine, Benzoisothiazolinone, Amylase, Sodium Citrate, Sodium Hydroxide, Fragrance.

### Tide to Go Stain Eraser:

Water, Alkyl Amine Oxide, Dipropylene Glycol Phenyl Ether, Hydrogen Peroxide, Citric Acid, Ethylene Diamine Disuccinic Acid Sodium salt, Sodium Alkyl Sulfate, Fragrance.

Tide Boost with Oxi:

Sodium bicarbonate, sodium carbonate, sodium percarbonate, alcohol ethoxylate, sodium chloride, maleic/acrylic copolymer, nonanoyloxy benzene sulfonate, sodium sulfate, colorant, diethylenetriamine pentaacetate sodium salt, hydrated aluminosilicate (zeolite), polyethylene glycol, sodium alkylbenzene sulfonate, sodium palmitate, starch, water, fragrance.

Tide Stain Release boost Duo Pac:

Polyvinyl Alcoholpouch film, wherein there is packed a liquid part and a powder part:

### Liquid Ingredients:

Dipropylene Glycol, diquaternium Ethoxysulfate, Water, Glycerin, Liquitint™ Orange, Powder Ingredients: sodium percarbonate, nonanoyloxy benzene sulfonate, sodium carbonate, sodium sulfate, sodium aluminosilicate, sodium polyacrylate, sodium alkylbenzenesulfonate, maleic/acrylic copolymer, water, amylase, polyethylene glycol, sodium palmitate, modified starch, protease, glycerine, DTPA, fragrance.

### Tide Ultra Stain Release:

Water, sodium alcoholethoxy sulfate, linear alkyl benzene sulfonate, sodium/MEA salts, MEA citrate, propylene glycol, polyethyleneimine ethoxylate, ethanol, diethylene glycol, polyethyleneimine propoxyethoxylate, sodium fatty acids, protease, borax, sodium cumene sulfonate, DTPA, fragrance, amylase, disodium diaminostilbene disulfonate, calcium formate, sodium formate, gluconase, dimethicone, Liquitint™ Blue, mannanase.

Ultra Tide with a Touch of Downy® Powdered Detergent, April Fresh/Clean Breeze/April Essence:

Sodium Carbonate, Sodium Aluminosilicate, Sodium Sulfate, Linear Alkylbenzene Sulfonate, Bentonite, Water, Sodium Percarbonate, Sodium Polyacrylate, Silicate, Alkyl Sulfate, Nonanoyloxybenzenesulfonate, DTPA, Polyethylene Glycol 4000, Silicone, Ethoxylate, fragrance, Polyethylene Oxide, Palmitic Acid, Disodium Diaminostilbene Disulfonate, Protease, Liquitint™ Red, FD&C Blue 1, Cellulase.

Ultra Tide with a Touch of Downy Clean Breeze:

Water, sodium alcoholethoxy sulfate, MEA citrate, linear alkyl benzene sulfonate: sodium/MEA salts, propylene glycol, polyethyleneimine ethoxylate, ethanol, diethylene glycol, polyethyleneimine, propoxyethoxylate, diquaternium ethoxysulfate, alcohol sulfate, dimethicone, fragrance, borax, sodium fatty acids, DTPA, protease, sodium bisulfite, disodium diaminostilbene disulfonate, amylase, gluconase, castor oil, calcium formate, MEA, styrene acrylate copolymer, sodium formate, Liquitint™ Blue.

Ultra Tide with Downy Sun Blossom:

Water, sodium alcoholethoxy sulfate, MEA citrate, linear alkyl benzene sulfonate: sodium/MEA salts, propylene glycol, ethanol, diethylene glycol, polyethyleneimine propoxyethoxylate, polyethyleneimine ethoxylate, alcohol sulfate, dimethicone, fragrance, borax, sodium fatty acids, DTPA, protease, sodium bisulfite, disodium diaminostilbene disulfonate, amylase, castor oil, calcium formate, MEA, styrene acrylate copolymer, propanaminium propanamide, gluconase, sodium formate, Liquitint™ Blue.

Ultra Tide with Downy April Fresh/Sweet Dreams:

Water, sodium alcoholethoxy sulfate, MEA citrate, linear alkyl benzene sulfonate: sodium/MEA salts, propylene glycol, polyethyleneimine ethoxylate, ethanol, diethylene glycol, polyethyleneimin propoxyethoxylate, diquaternium ethoxysulfate, alcohol sulfate, dimethicone, fragrance, borax, sodium fatty acids, DTPA, protease, sodium bisulfite, disodium diaminostilbene disulfonate, amylase, gluconase, castor oil, calcium formate, MEA, styrene acrylate copolymer, propanaminium propanamide, sodium formate, Liquitint™ Blue.

### Ultra Tide Free Powdered Detergent:

Sodium Carbonate, Sodium Aluminosilicate, Alkyl Sulfate, Sodium Sulfate, Linear Alkylbenzene Sulfonate, Water, Sodium polyacrylate, Silicate, Ethoxylate, Sodium percarbonate, Polyethylene Glycol 4000, Protease, Disodium Diaminostilbene Disulfonate, Silicone, Cellulase.

### Ultra Tide Powdered Detergent, Clean Breeze/Spring Lavender/Mountain Spring:

Sodium Carbonate, Sodium Aluminosilicate, Sodium Sulfate, Linear Alkylbenzene Sulfonate, Alkyl Sulfate, Sodium Percarbonate, Water, Sodium Polyacrylate, Silicate, Nonanoyloxybenzenesulfonate, Ethoxylate, Polyethylene Glycol 4000, Fragrance, DTPA, Disodium Diaminostilbene Disulfonate, Palmitic Acid, Protease, Silicone, Cellulase.

### Ultra Tide HE (High Efficiency) Powdered Detergent, Clean Breeze:

Sodium Carbonate, Sodium Aluminosilicate, Sodium Sulfate, Linear Alkylbenzene Sulfonate, Water, Nonanoyloxybenzenesulfonate, Alkyl Sulfate, Sodium Polyacrylate, Silicate, Sodium Percarbonate, Ethoxylate, Polyethylene Glycol 4000, Fragrance, DTPA, Palmitic Acid, Disodium Diaminostilbene Disulfonate, Protease, Silicone, Cellulase.

### Ultra Tide Coldwater Powdered Detergent, Fresh Scent:

Sodium Carbonate, Sodium Aluminosilicate, Sodium Sulfate, Sodium Percarbonate, Alkyl Sulfate, Linear Alkylbenzene Sulfonate, Water, Nonanoyloxybenzenesulfonate, Sodium Polyacrylate, Silicate, Ethoxylate, Polyethylene Glycol 4000, DTPA, Fragrance, Natalase, Palmitic Acid, Protease, Disodium, Diaminostilbene Disulfonate, FD&C Blue 1, Silicone, Cellulase, Alkyl Ether Sulfate.

Ultra Tide with Bleach Powdered Detergent, Clean Breeze:

Sodium Carbonate, Sodium Aluminosilicate, Sodium Sulfate, Linear Alkylbenzene Sulfonate, Sodium Percarbonate, Nonanoyloxybenzenesulfonate, Alkyl Sulfate, Water, Silicate, Sodium Polyacrylate, Ethoxylate, Polyethylene Glycol 4000, Fragrance, DTPA, Palmitic Acid, Protease, Disodium Diaminostilbene Disulfonate, Silicone, FD&C Blue 1, Cellulase, Alkyl Ether Sulfate.

Ultra Tide with Febreeze Freshness™ Powdered Detergent, Spring Renewal:

Sodium Carbonate, Sodium Aluminosilicate, Sodium Sulfate, Linear Alkylbenzene Sulfonate, Sodium Percarbonate, Alkyl Sulfate, Water, Sodium Polyacrylate, Silicate, Nonanoyloxybenzenesulfonate, Ethoxylate, Polyethylene Glycol 4000, DTPA, Fragrance, Cellulase, Protease, Disodium Diaminostilbene Disulfonate, Silicone, FD&C Blue 1.

Liquid Tide Plus with Febreeze Freshness—Sport HE Active Fresh:

Water, Sodium alcoholethoxy sulfate, MEA citrate, linear alkylbenzene sulfonate, sodium salt, linear alkylbenzene sulfonate: MEA salt, alcohol ethoxylate, sodium fatty acids, propylene glycol, diethylene glycol, polyethyleneimine ethoxylate propoxylate, diquaternium ethoxysulfate, Ethanol, sodium cumene sulfonate, borax, fragrance, DTPA, Sodium bisulfate, disodium diaminostilbene disulfonate, Mannanase, cellulase, amylase, sodium formate, calcium formate, Lauramine oxide, Liquitint™ Blue, Dimethicone/polydimethyl silicone.

### Tide Plus Febreeze Freshness Spring & Renewal:

Water, sodium alcoholethoxy sulfate, linear alkyl benzene sulfonate: sodium/MEA salts, MEA citrate, propylene glycol, polyethyleneimine ethoxylate, fragrance, ethanol, diethylene glycol, polyethyleneimine propoxyethoxylate, protease, alcohol sulfate, borax, sodium fatty acids, DTPA, disodium diaminostilbene disulfonate, MEA, mannanase, gluconase, sodium formate, dimethicone, Liquitint™ Blue, tetramine.

Liquid Tide Plus with Febreeze Freshness, Sport HE Victory Fresh:

Water, Sodium alcoholethoxy sulfate, MEA citrate, linear alkylbenzene sulfonate, sodium salt, linear alkylbenzene sulfonate: MEA salt, alcohol ethoxylate, sodium fatty acids, propylene glycol, diethylene glycol, polyethyleneimine ethoxylate propoxylate, diquaternium ethoxysulfate, ethanol, sodium cumene sulfonate, borax, fragrance, DTPA, Sodium bisulfate, disodium diaminostilbene disulfonate, Mannanase, cellulase, amylase, sodium formate, calcium formate, Lauramine oxide, Liquitint™ Blue, Dimethicone/polydimethyl silicone.

### Tide Vivid White+Bright Powder, Original:

Sodium Carbonate, Sodium Aluminosilicate, Sodium Sulfate, Linear Alkylbenzene Sulfonate, Sodium Percarbonate, Nonanoyloxybenzenesulfonate, Alkyl Sulfate, Water, Silicate, Sodium Polyacrylate Ethoxylate, Polyethylene Glycol 4000, Fragrance, DTPA, Palmitic Acid, Protease, Disodium Diaminostilbene Disulfonate, Silicone, FD&C Blue 1, Cellulase, Alkyl Ether Sulfate.

All percentages and ratios are calculated by weight unless otherwise indicated. All percentages and ratios are calculated based on the total composition unless otherwise indicated. It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Wash Tests

The following test setup (materials and wash tests) can used to evaluate the effect of the enzymes.

### Material

### Soil

W-SBL 2004, Soil Ballast Load Fabric purchased from CFT (Center for Testmaterials BV). WFK09v pigment soil purchased from CFT (Center for Testmaterials BV).

### Textile

TABLE 1
White tracer list

| No. | Name | Producer | Fabric |
|---|---|---|---|
| 1 | W-10 A | CFT | WFK standard cotton |
| 2 | W-12 A | CFT | Cotton terry |
| 3 | W-80 A | CFT | knitted cotton |
| 4 | C-N-11 | CFT | bleached woven cotton |
| 5 | C-N-42 | CFT | Cotton interlock double jersey |
| 6 | T-266 | CFT | Spun viscose challis |
| 7 | T-266 with | CFT | Spun viscose challis |
| | pre-aged treatment | | |
| 10 | P-CN-01 | CFT | Polyester/cotton, 65/35, woven |
| 11 | W-20 A | CFT | Polyester/cotton, 65/35, woven |
| 12 | T-720 | CFT | Texturized dacron 56T, Double knit |
| | | | jersey (disperse dyeable) |
| 13 | P-N-01 | CFT | Bleached woven polyester |
| 14 | W-30 A | CFT | polyester |
| 15 | W-40 A | CFT | Polyamid |
| 16 | T-340 Nylon/ | CFT | Nylon/Lycra, 81/19 |
| | Lycra, 81/19 | | |

CFT is abbreviation for “Center for Testmaterials BV”
Tracers are grouped in three categories for summarization of results:
Natural textile: W-10 A; W-12 A; W-80 A; C-N-11; C-N-42; T-266; T-266 with pre-aged treatment
Semisynthetic textile: P-CN-01; W-20 A
Synthetic textile: T-720; P-N-01; W-30 A; W-40 A; T-340 Nylon/Lycra 81/19
Real item refers to clothes or fabric used/worn by volunteer and not washed before FSW test.

TABLE 2
Real items used in Full scale wash (FSW) test

| Item | Resource | Textile type | Measurement |
|---|---|---|---|
| Used T-shirt | WARWICK EQUEST | Cotton | Remission at 460 |
| #1 | LIMITED | | nm |
| Used towel | Donated by volunteer | Cotton | Remission at 460 |
| | | | nm |
| Used T-shirt | Donated by volunteer | Cotton | Remission at 460 |
| #2 | | | nm |
| Used T-shirt | Donated by volunteer | Polyester | Remission at 460 |
| #3 | | | nm |
| Used Shirt | Donated by volunteer | Cotton/Polyester | Remission at 460 |
| | | | nm |
| Pillowcase | Donated by volunteer | Cotton | Remission at 460 |
| | | | nm |
| Item | Resource | Textile type | Measurement |
| Used | Taiyuan Sinolight | Cotton | Panel test |
| Shirt #2 | Surfactants | | |
| | Technology CO., Ltd | | |
| Used Socks | Taiyuan Sinolight | Cotton | Panel test |
| #1 | Surfactants | | |
| | Technology CO., Ltd | | |
| Used Socks | WARWICK EQUEST | Cotton | Panel test |
| #2 | LIMITED | | |
| Used Socks | Donated by volunteer | Cotton | Panel test |
| #3 | | | |

TABLE 3
Detergent B (wt %)

| | |
|---|---|
| LAS, (C10-C13) alkylbenzene-sulfonic acid | 3.8% |
| AES, AEOS, sodium lauryl ether sulfate | 8% |
| AEO, Alcohol ethoxylate | 4% |
| Soap, lauric acid | 1.0% |
| Trisodium citrate dihydrate | 2% |
| Sodium hydroxide | 0.6% |
| CaCl2, 2H2O | 0.02% |
| Kathon, preservative | 0.1% |
| Triethanolamine | 0.4% |
| Deionized Water to 100% | Ad 100% |

### Full Scale Wash (FSW) Assay for Whiteness on Real Item (Used Item)

FSW is used to evaluate wash performance in washing machines under scientifically designed conditions.
Wash conditions: Standard EU washing conditions are described below.

TABLE 4
Standard EU washing conditions

| | EU washing machine | | |
|---|---|---|---|
| | Miele WPS W5841 | Description | |
| Cleaning | Wash program | Cotton/short | |
| wash | Water level (water plus) | 15.6 L with Water plus | |
| | Ballast | 4 kg total weight (including | |
| | | swatches and washload). | |
| | | Mixed cotton/polyester ratio at 65/35 | |
| | Temperature | 40° C. | |
| | Wash time | 51 min main wash and 3 cycles of rinse | |
| | Water hardness | 15° dH. Ca²⁺/Mg²⁺/HCO₃⁻ Ca²⁺/Mg²⁺/HCO₃⁻ | |
| | | ratio 4:1:7.5 | |
| | Detergent | Detergent and dosage: according to the | |
| | | study | |
| | Enzyme | Used | |
| | Test swatches | White tracers | |
| | Soil | No | |
| | Wash cycle | 1 | cycle |
| Soil | Wash program | Express 20, no short, no water plus | |
| rinse | Water level | 7.5 | L |
| | Water hardness | 15° dH. Ca²⁺/Mg²⁺/HCO₃⁻ Ca²⁺/Mg²⁺/HCO₃⁻ | |
| | | ratio 4:1:7.5 | |
| | Wash time | 30 | min |

| | |
|---|---|
| Detergent | 7.5 g/wash detergent B |
| Soil | 7.5 g/wash pigment soil (WFK09v) |

The wash procedure instructions below are applied:
a. Prepare the ballast and test swatches, and hard water with Ca/Mg according to desired water hardness. Select the core part the real item and cut evenly into 2 or 4 pieces. Please note the stains, yellowish and graying should be evenly distributed into each piece.
b. Add ballast and cut real item piece to wash machine. Each piece from one real item is randomly add to each test conditions respectively.
c. Dissolve detergent in 1 L hardwater and stir for 30 min.
d. Select parameters for the wash: Program, Water level and Temperature.
e. Press start button of machine to start water filling. Water consumption is registered automatically during this time.
j. Add in detergent solution through detergent tank and rinse the beaker with hard water and add rinse water into washing machine to ensure all the detergent is added into machine drum.
f. After the wash is completed, remove the ballast and leave the real item pieces in wash machine.
g. Add 7.5 g Detergent B and 7.5 g pigment soil into 1 L hard water (14 dH as it is in main wash), and stir for 10 min.
h. Select parameters for soil rinse: Program and Water level.
i. Add in Detergent B pigment soil solution through detergent tank after water is intaken automatically. Rinse the beaker with hard water for several times and add rinse water into washing machine.
j. After the wash is completed, the test swatches are removed from the tea towels and placed on trays for drying.

### Terg-O-Tometer (TOM) Wash Assay

The Tergo-To-Meter (TOM) is a medium scale model wash system that can be applied to test 16 different wash conditions simultaneously. A TOM is basically a large temperature-controlled water bath with up to 16 open metal beakers submerged into it. Each beaker constitutes one small top loader style washing machine and during an experiment, each of them will contain a solution of a specific detergent/enzyme/polymer system and the soiled and unsoiled fabrics its performance is tested on. Mechanical stress is achieved by a rotating stirring arm, which stirs the liquid within each beaker.

The TOM model wash system is mainly used in medium scale testing of detergents, enzymes and polymers at EU or AP wash conditions. In a TOM experiment, factors such as the ballast to soil ratio and the fabric to wash liquor ratio can be varied. Therefore, the TOM provides the link between small scale experiments, and the more time-consuming full-scale experiments.

Equipment: The water bath with 16 steel beakers and 1 rotating arm per beaker with capacity of 1 L detergent solution. Temperature ranges from 5° C. to 80° C. The water bath has to be filled up with deionised water. Rotational speed can be set up to 70 to 120 rpm/min.

Set temperature in the Terg-0-Tometer and start the rotation in the water bath. Wait for the temperature to adjust (tolerance is +/−0.5° C.). All beakers shall be clean and without traces of prior test material.

The wash solution with desired amount of detergent, temperature and water hardness is prepared in a bucket. The detergent is allowed to dissolve during magnet stirring for 10 min. Wash solution shall be used within 30 to 60 min after preparation.

1 L wash solution is added into a TOM beaker. The wash solution is agitated at 120 rpm and optionally one or more enzymes or polymers are added to the beaker. The swatches are sprinkled into the beaker and then the ballast load. Time measurement starts when the swatches and ballast are added to the beaker. The swatches are washed for 20 or 30 minutes after which agitation is terminated.

The wash load is subsequently transferred from the TOM beaker to a sieve and rinse with cold tap water. The soil swatches are separated from the ballast load. The soil swatches are transferred to a 5 L beaker with cold tap water under running water for 5 minutes. The ballast load is kept separately for the coming inactivation. The water is gently pressed out of the swatches by hand and placed on a tray covered with a paper. The swatches are allowed to dry overnight before subjecting the swatches to analysis, such as measuring the delta REM.

### Whiteness Panel on Real Items

Panel test is built on visual whiteness assessment by 8 panelists. To increase the panel differentiation, real items are cut into 2 equal pieces and washed by 2 conditions which is compared in pair.
Panelists are asked to give their preference according to cleaning appearance of each real item after wash in pair. Moreover, give their panel score according to following criteria:

| Degree | Score |
|---|---|
| I think this condition better | 1 |
| I know this condition better | 2 |
| I confirm this condition better | 3 |
| I confirm this much better | 4 |

When a test condition and a benchmark are compared, a positive score means the test condition looks better/brighter/cleaner than benchmark, and a negative score indicates the test condition is inferior/darker/less clean than benchmark. The benchmark is decided in the trial and will be indicated in result presentation.
Preference % is the percentage of the panelists who prefer the test condition (in this trial the number of panelists who prefer the test condition over the benchmark divided by total of 8 panelists, calculated into %).

Average of Confidence=Σ(panel score on each item).

### Light Reflectance Measurement (Delta REM)

After washing and rinsing the swatches are spread out flat and allowed to air dry at room temperature overnight. All washes are evaluated the day after the wash. Brightness can also be expressed as the Remission (R), which is a measure for the light reflected or emitted from the test material when illuminated with white light. The Remission (R) of the textiles is measured at 460 nm using a Macbeth Color Eye 7000 reflectance spectrophotometer with very small aperture The measurements are made without UV in the incident light and remission at 460 nm is extracted. The measurements are done per the manufacturer's protocol.

| | A | B | C | D |
|---|---|---|---|---|
| Detergent | Ad 100% | Ad 100% | Ad 100% | Ad 100% |
| Betaglucanase | — | 0.01-1000 ppm | — | 0.01- |
| SEQ ID NO: 1-5 | | | | 1000ppm |
| DNAse | — | — | 0.01- | 0.01- |
| SEQ ID NO: 1-5 | | | 1000 ppm | 1000 ppm |

Detergent could be any detergent, powder, liquid, unit dose, gel, etc. with optional additives and other enzymes and is added the enzymes of the invention and washed with test #1 or TOM test and evaluated by panel score and delta REM.

1. An enzyme composition comprising
a) a polypeptide having beta-glucanase activity and having at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the sequence selected from the group consisting of: SEQ ID NO: 1, SEQ ID NO: 2, SEQ ID NO: 3, SEQ ID NO: 4, SEQ ID NO: 5; and
b) a polypeptide having DNase activity and having at least 60%, at least 65%, at least 70%, at least 75%, at least 80%, at least 85%, at least 90%, at least 95%, at least 98% or 100% sequence identity to the DNase shown in SEQ ID NO: 6 or SEQ ID NO: 7.

2. The composition of claim 1, wherein the beta-glucanase has at least 60%, at least 65%, at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, at least 99% or 100% sequence identity to the beta-glucanase shown in SEQ ID NO: 1.

3. The composition of claim 1, wherein the beta-glucanase is selected from the group consisting of a polypeptide having amino acids 1 to 222 of SEQ ID NO: 1, amino acids 1 to 351 of SEQ ID NO: 2, amino acids 1 to 351 of SEQ ID NO: 3, amino acids 1 to 245 of SEQ ID NO: 4, amino acids 1 to 214 of SEQ ID NO: 5.

4. The composition according to of claim 1, wherein the DNase is obtained from Bacillus, preferably Bacillus cibi or is obtained from Aspergillus, preferably Aspergillus oryzae.

5. The composition according to of claim 1 wherein the amount of beta-glucanase in the composition is from 0.01 to 1000 ppm and the amount of DNase is from 0.01 to 1000 ppm.

6. The composition of claim 1 which is a cleaning composition comprising one or more cleaning component, wherein the cleaning component is selected from a surfactant, preferably anionic and/or nonionic, builder and bleach component.

7. The composition according to of claim 1, wherein the beta-glucanase provides at least one enzyme detergency benefit.

8. The composition according to of claim 1, further comprising at least one additional enzyme selected from the group consisting of amylase, cellulase, lipase, mannanase, pectinase, and protease, or a combination thereof, preferably wherein the amylase, cellulase, lipase, mannanase, pectinase, and protease, or a combination thereof, provide at least one enzyme detergency benefit.

9. The composition according to of claim 8, wherein the additional enzyme is an amylase, and wherein the amylase is selected from the group consisting of;
a) an amylase having at least 60%, at least 65%, at least 70%, at least 75%, at least 80%, at least 85%, at least 90%, at least 95%, at least 98% or 100% sequence identity to SEQ ID NO: 8 or SEQ ID NO: 41, or an amylase having at least 60%, at least 65%, at least 70%, at least 75%, at least 80%, at least 85%, at least 90%, at least 95%, at least 98% but less than 100% sequence identity to SEQ ID NO: 8 or SEQ ID NO: 41 comprising a two amino acid deletion in the sequence region R180, S181, T182, G183, compared to SEQ ID NO: 8, wherein each position corresponds to the position in SEQ ID NO: 8;
b) an amylase having at least 60%, at least 65%, at least 70%, at least 75%, at least 80%, at least 85%, at least 90%, at least 95%, at least 98% or 100% sequence identity to SEQ ID NO: 8 or SEQ ID NO: 41, or an amylase having at least 60%, at least 65%, at least 70%, at least 75%, at least 80%, at least 85%, at least 90%, at least 95%, at least 98% but less than 100% sequence identity to SEQ ID NO: 8 or SEQ ID NO: 41 comprising one of the alterations set selected from the group consisting of:
a. R180*, S181*, S243Q, G475K;
b. R180*, T182*, S243Q, G475K;
c. R180*, T182*, G183S, S243Q, G475K; and
d. R180*, S181*, Y242F, S243Q, F266Y, G475K compared to SEQ ID NO: 8, wherein each position corresponds to the position in SEQ ID NO: 8;
c) an amylase having at least 60%, at least 65%, at least 70%, at least 75%, at least 80%, at least 85%, at least 90%, at least 95%, at least 98% or 100% sequence identity to SEQ ID NO: 9, or an amylase having at least 60%, at least 65%, at least 70%, at least 75%, at least 80%, at least 85%, at least 90%, at least 95%, at least 98% but less than 100% sequence identity to SEQ ID NO: 9, comprising a two amino acid deletion in the sequence region R178, G179, T180, G181 compared to SEQ ID NO: 9, wherein each position corresponds to the position in SEQ ID NO: 9;
d) an amylase having at least 60%, at least 65%, at least 70%, at least 75%, at least 80%, at least 85%, at least 90%, at least 95%, at least 98% or 100% sequence identity to SEQ ID NO: 9, or an amylase having at least 60%, at least 65%, at least 70%, at least 75%, at least 80%, at least 85%, at least 90%, at least 95%, at least 98% but less than 100% sequence identity to SEQ ID NO: 9, comprising one of the alterations set selected from the group consisting of:
I. R178*, G179*, E187P, I203Y, G476K;
II. R178*, G179*, E187P, M199L, I203Y, G476K;
III. R178*, G179*, E187P, I203Y R458N, T459S, D460T, G476K;
IV. N126Y, F153W, R178*, G179*, T180H, I203Y, S241Q;
V. N126Y, F153W, R178*, G179*, T180H, I203Y, S241Q, S362A, R377Y;
VI. T38N, N126Y, T1291, F153W, R178*, G179*, T180D, E187P, I203Y, G476K, G477E; and
VII. N126Y, F153W, R178*, G179*, T180H, E187P, I203Y, S241Q, G476K, G477E, compared to SEQ ID NO: 9, wherein each position corresponds to the position in SEQ ID NO: 9;
e) an amylase having at least 60%, at least 65%, at least 70%, at least 75%, at least 80%, at least 85%, at least 90%, at least 95%, at least 98% or 100% sequence identity to SEQ ID NO: 10, or an amylase having at least 60%, at least 65%, at least 70%, at least 75%, at least 80%, at least 85%, at least 90%, at least 95%, at least 98% but less than 100% sequence identity to SEQ ID NO: 10, comprising a two amino acid deletion in the sequence region R181, G182, D183, G184 compared to SEQ ID NO: 10, wherein each position corresponds to the position in SEQ ID NO: 10;
f) an amylase having at least 60%, at least 65%, at least 70%, at least 75%, at least 80%, at least 85%, at least 90%, at least 95%, at least 98% or 100% sequence identity to SEQ ID NO: 10, or an amylase having at least 60%, at least 65%, at least 70%, at least 75%, at least 80%, at least 85%, at least 90%, at least 95%, at least 98% but less than 100% sequence identity to SEQ ID NO: 10 comprising an alteration at one or more, preferably at all of the position(s) selected from 3, 4, 5, 74, 118, 167, 170, 177, 195, 202, 204, 271, 320, 330, 377, 385, 445, 458, 475, 476, 314, 315 or 316, compared to SEQ ID NO: 10, wherein each position corresponds to the position in SEQ ID NO: 10;
g) an amylase having at least 60%, at least 65%, at least 70%, at least 75%, at least 80%, at least 85%, at least 90%, at least 95%, at least 98% or 100% sequence identity to SEQ ID NO: 11, or an amylase having at least 60%, at least 65%, at least 70%, at least 75%, at least 80%, at least 85%, at least 90%, at least 95%, at least 98% but less than 100% sequence identity to SEQ ID NO: 11 preferably comprising a two amino acid deletion in the sequence region R181, G182, D183, G184, compared to SEQ ID NO: 11, wherein each position corresponds to the position in SEQ ID NO: 11;
h) an amylase having at least 60%, at least 65%, at least 70%, at least 75%, at least 80%, at least 85%, at least 90%, at least 95%, at least 98% or 100% sequence identity to SEQ ID NO: 11, or an amylase having at least 60%, at least 65%, at least 70%, at least 75%, at least 80%, at least 85%, at least 90%, at least 95%, at least 98% but less than 100% sequence identity to SEQ ID NO: 11 comprising one of the alterations set selected from the group consisting of
a. D183*, G184*, N195F, Y243F;
b. D183*, G184*, N195F, V206Y, Y243F;
c. W140Y, D183*, G184*, N195F, V206Y, Y243F, E260G, G304R, G476K;
d. W140Y, D183*, G184*, N195F, V206Y, Y243F, E260G, G477E;
e. W140Y, D183*, G184*, N195F, V206Y, Y243F, W284D;
f. W140Y, N195F, V206Y, Y243F, E260G, G477E;
g. G109A, W140Y, N195F, V206Y, Y243F, E260G;
h. T51I, S52Q, N54K, G109A, W140Y, N195F, V206Y, Y243F, E260G, G476E;
i. W140Y, N195F, V206Y, Y243F, E260G, W284R, G477K;
j. W140Y, N195F, V206Y, Y243F, E260G, W284F, G477R; and
k. H1*, G7A, G109A, W140Y, D183*, G184*, N195F, V206Y, Y243F, E260G, N280S, G304R, E391A, G476K, compared to SEQ ID NO: 11, wherein each position corresponds to the position in SEQ ID NO: 11;
i) an amylase having at least 60%, at least 65%, at least 70%, at least 75%, at least 80%, at least 85%, at least 90%, at least 95%, at least 98% or 100% sequence identity to SEQ ID NO 6, or an amylase having at least 60%, at least 65%, at least 70%, at least 75%, at least 80%, at least 85%, at least 90%, at least 95%, at least 98% but less than 100% sequence identity to SEQ ID NO: 12, comprising a two amino acid deletion in the sequence region R181, G182, D183, G184, compared to SEQ ID NO: 12, wherein each position corresponds to the position in SEQ ID NO: 12;
j) an amylase having at least 60%, at least 65%, at least 70%, at least 75%, at least 80%, at least 85%, at least 90%, at least 95%, at least 98% or 100% sequence identity to SEQ ID NO: 12, or an amylase having at least 60%, at least 65%, at least 70%, at least 75%, at least 80%, at least 85%, at least 90%, at least 95%, at least 98% but less than 100% sequence identity to SEQ ID NO: 12, comprising one of the alterations set selected from the group consisting of
I. R118K, D183*, G184*, N195F, R320K, R458K;
II. M9I, D183*, G184*, R118K, N195F, M202L, R320K, M323T, R458K;
III. M9L, G149A, R118K, G182T, D183*, G184*, G186A, N195F, M202L, T257I, Y295F, N299Y, M323T, A339S, E345R, R458K;
IV. M9L, G149A, R118K, G182T, D183*, G184*, G186A, N195F, T246V, T257I, Y295F, N299Y, M323T, A339S, E345R, R458K; and
V. M9L, G149A, G182T, D183*, G184*, G186A, M202L, T257I, Y295F, N299Y, M323T, A339S, E345R, N471E, compared to SEQ ID NO: 12, wherein each position corresponds to the position in SEQ ID NO: 12;
k) an amylase having at least 60%, at least 65%, at least 70%, at least 75%, at least 80%, at least 85%, at least 90%, at least 95%, at least 98% or 100% sequence identity to SEQ ID NO: 13, or an amylase having at least 60%, at least 65%, at least 70%, at least 75%, at least 80%, at least 85%, at least 90%, at least 95%, at least 98% but less than 100% sequence identity to SEQ ID NO: 13, comprising a two amino acid deletion in the sequence region R181, G182, D183, G184, compared to SEQ ID NO: 13, wherein each position corresponds to the position in SEQ ID NO: 13;
l) an amylase having at least 60%, at least 65%, at least 70%, at least 75%, at least 80%, at least 85%, at least 90%, at least 95%, at least 98% or 100% sequence identity to SEQ ID NO: 13, or an amylase having at least 60%, at least 65%, at least 70%, at least 75%, at least 80%, at least 85%, at least 90%, at least 95%, at least 98% but less than 100% sequence identity to SEQ ID NO: 13, comprising one of the alterations set selected from the group consisting of
a. D183*, G184*, N195F, V206Y, R320K, R458K;
b. D183*, G184*, N195F, M202L, V206L, R320K, R458K;
c. G149A, G182T, D183*, G184*, N195F, M202L, V206L, T257I, Y295F, Q299Y, A339S, Q345R, R458K;
d. G149A, G182T, D183*, G184*, N195F, V206L, M246V, T257I, Y295F, Q299Y, A339S, Q345R, R458K;
e. G149A, G182T, D183*, G184*, M202L, V206L, T257I, Y295F, Q299Y, A339S, Q345R, H₄₇₁E; and
f. H₁A, N54S, V56T, K72R, G109A, F113Q, R116Q, W167F, Q172G, A174S, G182*, D183*, G184T, N195F, V206L, K391A, F473R, G476K, compared to SEQ ID NO: 13, wherein each position corresponds to the position in SEQ ID NO: 13;
m) an amylase having at least 60%, at least 65%, at least 70%, at least 75%, at least 80%, at least 85%, at least 90%, at least 95%, at least 98% or 100% sequence identity to SEQ ID NO: 14, or an amylase having at least 60%, at least 65%, at least 70%, at least 75%, at least 80%, at least 85%, at least 90%, at least 95%, at least 98% but less than 100% sequence identity to SEQ ID NO: 14, comprising a two amino acid deletion in the sequence region R181, G182, H183, G184, compared to SEQ ID NO: 14, wherein each position corresponds to the position in SEQ ID NO: 14;
n) an amylase having at least 60%, at least 65%, at least 70%, at least 75%, at least 80%, at least 85%, at least 90%, at least 95%, at least 98% or 100% sequence identity to SEQ ID NO: 14, or an amylase having at least 60%, at least 65%, at least 70%, at least 75%, at least 80%, at least 85%, at least 90%, at least 95%, at least 98% but less than 100% sequence identity to SEQ ID NO: 14, comprising one of the alterations set selected from the group consisting of
a. H183*, G184*, I405L, A421H, A422P, A428T;
b. R118K, H183*, G184*, N195F, R320K, R458K;
c. M9I, H183*, G184*, R118K, N195F, M202L, R320K, S323T, R458K;
d. M9L, G149A, R118K, G182T, H183*, G184*, N195F, M202L, T257I, Y295F, N299Y, A339S, E345R, R458K;
e. M9L, G149A, R118K, G182T, H183*, G184*, N195F, T246V, T257I, Y295F, N299Y, A339S, E345R, R458K; and
f. M9L, G149A, G182T, H183*, G184*, M202L, T257I, Y295F, N299Y, S323T, A339S, E345R, compared to SEQ ID NO: 14, wherein each position corresponds to the position in SEQ ID NO: 14;
o) an amylase having at least 60%, at least 65%, at least 70%, at least 75%, at least 80%, at least 85%, at least 90%, at least 95%, at least 98% or 100% sequence identity to SEQ ID NO: 15, or an amylase having at least 60%, at least 65%, at least 70%, at least 75%, at least 80%, at least 85%, at least 90%, at least 95%, at least 98% but less than 100% sequence identity to SEQ ID NO: 15, comprising a two amino acid deletion in the sequence region R181, G182, G182, D183, compared to SEQ ID NO: 15, wherein each position corresponds to the position in SEQ ID NO: 15;
p) an amylase having at least 60%, at least 65%, at least 70%, at least 75%, at least 80%, at least 85%, at least 90%, at least 95%, at least 98% or 100% sequence identity to SEQ ID NO: 15, or an amylase having at least 60%, at least 65%, at least 70%, at least 75%, at least 80%, at least 85%, at least 90%, at least 95%, at least 98% but less than 100% sequence identity to SEQ ID NO: 15, comprising one of the alterations set selected from the group consisting of
a. H1*, D183*, G184*, N195F, V206Y;
b. H1*, D183*, G184*, N195F, M202L, V206L, R320K, R458K;
c. G149A, G182T, D183*, G184*, N195F, M202L, V206L, T257I, Y295F, Q299Y, A339S, Q345R, R458K;
d. G149A, G182T, D183*, G184*, N195F, V206L, M246V, T257I, Y295F, Q299Y, A339S, Q345R, R458K;
e. G149A, G182T, D183*, G184*, M202L, V206L, T257I, Y295F, Q299Y, A339S, Q345R,
f. H1*, N54S, V56T, G109A, Q169E, Q172K, A174*, G182*, D183*, N195F, V206L, K391A, G476K;
g. G182*, D183*, N195F, W140Y, N260G, 5304R, R320A, G476K, V410I, V429I, F451W, C474V;
h. H1*, N54S, V56T, K72R, G109A, F113Q, R116Q, W167F, Q172G, A174S, G182*, D183*, G184T, N195F, V206L, K391A, P473R, G476K;
i. H1*, N54S, V56T, G109A, Q169E, Q172K, A174*, G182*, D183*, N195F, V206L, K391A, G476K;
j. H1*, N54S, V56T, G109A, R116H, A174S, G182*, D183*, N195F, V206L, K391A, G476K;
k. H1*, N54S, V56T, K72R, G109A, F113Q, R116Q, W167F, Q172G, A174S, G182*, D183*, G184T, N195F, V206L, K391A, P473R, G476K;
l. H1*, N54S, V56T, G109A, F113Q, R116Q, Q172N, A174S, G182*, D183*, N195F, V206L, A265G, K391A, P473R, G476K;
m. H1*, N54S, V56T, K72R, G109A, F113Q, W167F, Q172R, A174S, G182*, D183*, N195F, V206L, K391A, G476K;
n. H1*, N54S, V56T, K72R, G109A, R116H, T134E, W167F, Q172G, L173V, A174S, G182*, D183*, N195F, V206L, G255A, K391A, G476K;
o. H1*, N54S, V56T, K72R, G109A, R116H, T134E, W167F, Q172G, L173V, A174S, G182*, D183*, N195F, V206L, G255A, K391A, Q395P, T444Q, P473R, G476K;
p. H1*, N54S, V56T, G109A, T134E, A174S, G182*, D183*, N195F, V206L, K391A, G476K;
q. H1*, N54S, V56T, K72R, G109A, A174S, G182*, D183*, N195F, V206L, G255A, K391A, G476K;
r. H1*, N54S, V56T, G109A, W167F, Q172E, L173P, A174K, G182*, D183*, N195F, V206L, K391A, G476K;
s. H1*, N54S, V56T, G109A, R116Q, V120L, Q172G, L173V, A174S, G182*, D183*, G184T, N195F, V206L, A422P; and
t. H1*, N54S, V56T, G109A, F113Q, R116Q, W167F, Q172G, 1173V, A174S, G182*, D183*, G184T, N195F, V206L, A422P compared to SEQ ID NO: 15, wherein each position corresponds to the position in SEQ ID NO: 15.

10. The composition of claim 8, wherein the additional enzyme is a cellulase, and wherein the cellulase is selected from the group consisting of;
a) a cellulase having at least 60%, at least 65%, at least 70%, at least 75%, at least 80%, at least 85%, at least 90%, at least 95%, at least 98% or 100% sequence identity to SEQ ID NO: 16;
b) a cellulase having at least 60%, at least 65%, at least 70%, at least 75%, at least 80%, at least 85%, at least 90%, at least 95%, at least 98% or 100% sequence identity to SEQ ID NO: 17;
c) a cellulase having at least 60%, at least 65%, at least 70%, at least 75%, at least 80%, at least 85%, at least 90%, at least 95%, at least 98% or 100% sequence identity to SEQ ID NO: 18;
d) a cellulase having at least 60%, at least 65%, at least 70%, at least 75%, at least 80%, at least 85%, at least 90%, at least 95%, at least 98% or 100% sequence identity to SEQ ID NO: 19,
e) a cellulase having at least 60%, at least 65%, at least 70%, at least 75%, at least 80%, at least 85%, at least 90%, at least 95%, at least 98% or 100% sequence identity to SEQ ID NO: 37, and
f) a cellulase having at least 60%, at least 65%, at least 70%, at least 75%, at least 80%, at least 85%, at least 90%, at least 95%, at least 98% or 100% sequence identity to SEQ ID NO: 38.

11. The composition of claim 8, wherein the additional enzyme is a lipase, and, wherein the lipase is a lipase having at least 60%, at least 65%, at least 70%, at least 75%, at least 80%, at least 85%, at least 90%, at least 95%, at least 98% or 100% sequence identity to SEQ ID NO: 20, or a lipase having at least 60%, at least 65%, at least 70%, at least 75%, at least 80%, at least 85%, at least 90%, at least 95%, at least 98% but less than 100% sequence identity to SEQ ID NO: 20 comprising one or more of the substitutions selected from the group consisting of D27R, G38A, G91A/Q, D96E, G163K, T231R, N233R, D254S and P256T, compared to SEQ ID NO: 20, wherein each position corresponds to the position in SEQ ID NO: 20.

12. The composition of claim 8, wherein the additional enzyme is a mannanase, and wherein the mannanase is selected from the group consisting of;
a) a mannanase, wherein the mannanase preferably belongs to the Glycoside Hydrolase Family 5 mannanases;
i. a mannanase having at least 60%, at least 65%, at least 70%, at least 75%, at least 80%, at least 85%, at least 90%, at least 95%, at least 98% or 100% sequence identity to SEQ ID NO: 21;
b) a mannanase wherein the mannanase preferably belongs to the Glycoside Hydrolase Family 26 mannanases;
i. a mannanase having at least 60%, at least 65%, at least 70%, at least 75%, at least 80%, at least 85%, at least 90%, at least 95%, at least 98% or 100% sequence identity to SEQ ID NO: 22;
ii. a mannanase having at least 60%, at least 65%, at least 70%, at least 75%, at least 80%, at least 85%, at least 90%, at least 95%, at least 98% or 100% sequence identity to SEQ ID NO: 23;
iii. a mannanase having at least 60%, at least 65%, at least 70%, at least 75%, at least 80%, at least 85%, at least 90%, at least 95%, at least 98% or 100% sequence identity to SEQ ID NO: 24;
iv. a mannanase having at least 60%, at least 65%, at least 70%, at least 75%, at least 80%, at least 85%, at least 90%, at least 95%, at least 98% or 100% sequence identity to SEQ ID NO: 25;
v. a mannanase having at least 60%, at least 65%, at least 70%, at least 75%, at least 80%, at least 85%, at least 90%, at least 95%, at least 98% or 100% sequence identity to SEQ ID NO: 26;
vi. a mannanase having at least 60%, at least 65%, at least 70%, at least 75%, at least 80%, at least 85%, at least 90%, at least 95%, at least 98% or 100% sequence identity to SEQ ID NO: 27;
vii. a mannanase having at least 60%, at least 65%, at least 70%, at least 75%, at least 80%, at least 85%, at least 90%, at least 95%, at least 98% or 100% sequence identity to SEQ ID NO: 28;
viii. a mannanase having at least 60%, at least 65%, at least 70%, at least 75%, at least 80%, at least 85%, at least 90%, at least 95%, at least 98% or 100% sequence identity to SEQ ID NO: 29;
ix. a mannanase having at least 60%, at least 65%, at least 70%, at least 75%, at least 80%, at least 85%, at least 90%, at least 95%, at least 98% or 100% sequence identity to SEQ ID NO: 30;
x. a mannanase having at least 60%, at least 65%, at least 70%, at least 75%, at least 80%, at least 85%, at least 90%, at least 95%, at least 98% or 100% sequence identity to SEQ ID NO: 39; and
xi. a mannanase having at least 60%, at least 65%, at least 70%, at least 75%, at least 80%, at least 85%, at least 90%, at least 95%, at least 98% or 100% sequence identity to SEQ ID NO: 40.

13. The composition of claim 8, wherein the additional enzyme is a protease, and wherein the protease is selected from the group consisting of;
a) a protease having at least 60%, at least 65%, at least 70%, at least 75%, at least 80%, at least 85%, at least 90%, at least 95%, at least 98%, at least 99% or 100% sequence identity to SEQ ID NO: 31, preferably obtained from Bacillus lentus;
b) a protease having at least 60%, at least 65%, at least 70%, at least 75%, at least 80%, at least 85%, at least 90%, at least 95%, at least 98%, at least 99% or 100% sequence identity to SEQ ID NO: 32; preferably obtained from Bacillus amyloliquefaciens;
c) a protease having at least 60%, at least 65%, at least 70%, at least 75%, at least 80%, at least 85%, at least 90%, at least 95%, at least 98%, at least 99% or 100% sequence identity to SEQ ID NO: 33, preferably obtained from Bacillus sp.;
d) a protease having at least 60%, at least 65%, at least 70%, at least 75%, at least 80%, at least 85%, at least 90%, at least 95%, at least 98%, at least 99% or 100% sequence identity to SEQ ID NO: 34, preferably obtained from Bacillus gibsonii;
e) a protease having at least 60%, at least 65%, at least 70%, at least 75%, at least 80%, at least 85%, at least 90%, at least 95%, at least 98%, at least 99% or 100% sequence identity to SEQ ID NO: 35, preferably obtained from Bacillus lentus;
f) a protease having at least 60%, at least 65%, at least 70%, at least 75%, at least 80%, at least 85%, at least 90%, at least 95%, at least 98%, at least 99% or 100% sequence identity to SEQ ID NO: 36, preferably obtained from Bacillus licheniformis;
g) or a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises the substitution T22R or T22A compared to SEQ ID NO: 31, wherein the position corresponds to the position of SEQ ID NO: 31;
h) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises one or more, preferably all the substitutions selected from the group consisting of: S3T, V41, A188P and V199I, compared to SEQ ID NO: 31, wherein the positions correspond to the positions of SEQ ID NO: 31;
i) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises one or more, preferably all the substitutions selected from the group consisting of N114L, T207A, A226V, and E265F, compared to SEQ ID NO: 31, wherein the positions correspond to the positions of SEQ ID NO: 31;
j) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises one or more, preferably all substitutions selected from the group consisting of: S97D, S101A, V102I and G157S compared to SEQ ID NO: 31, wherein the positions correspond to the positions of SEQ ID NO: 31;
k) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises one or more, preferably all the substitutions selected from the group consisting of: S85N, G116V, S126L, P127Q and S128A compared to SEQ ID NO: 31, wherein the positions correspond to the positions of SEQ ID NO: 31;
l) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises one or more, preferably all the substitutions selected from the group consisting of: Y161A, R164S and A188P, compared to SEQ ID NO: 31, wherein the positions correspond to the positions of SEQ ID NO: 31;
m) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises one or more, preferably all the substitutions selected from the group consisting of: S3T, R19L, and A188P, wherein the positions correspond to the positions of SEQ ID NO: 31;
n) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises one or more, preferably all the substitutions selected from the group consisting of: S9R, R19L, and N60D, wherein the positions correspond to the positions of SEQ ID NO: 31;
o) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises the amino acid Arginine (R), at a position corresponding to a position selected from the group consisting of: 9, 42 and 239 of SEQ ID NO: 31;
p) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises the amino acid Glutamic acid (E) or Aspartic acid (D), at a position corresponding to a position selected from the group consisting of: 9, 42, 60, 61, 74, 157, 176, 179, 182, 212, 250, 253 and 256 of SEQ ID NO: 31;
q) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises an insertion of the amino acid Aspartic acid (D) or Glutamic acid (E) at a position corresponding to position 97 of SEQ ID NO: 31;
r) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises the amino acid selected from the group consisting of: Glutamic acid (E), Aspartic acid (D), Glycine (G), Arginine (R) and Methionine (M) at a position corresponding to position 99 of SEQ ID NO: 31;
s) a protease having at least 60%, at least 65%, at least 70%, at least 75%, at least 80%, at least 85%, at least 90%, at least 95%, at least 98% but less than 100% sequence identity to SEQ ID NO: 31, wherein the protease comprises the amino acid selected from the group consisting of: Glutamic acid (E), Aspartic acid (D) and Glutamine (Q), at a position corresponding to position 211 of SEQ ID NO: 31;
t) a protease having at least 60%, at least 65%, at least 70%, at least 75%, at least 80%, at least 85%, at least 90%, at least 95%, at least 98% but less than 100% sequence identity to SEQ ID NO: 32, wherein the protease comprises the amino acid selected from the group consisting of: Glutamic acid (E), Aspartic acid (D) and Glutamine (Q), at a position corresponding to position 217 of SEQ ID NO: 32;
u) a protease having at least 60%, at least 65%, at least 70%, at least 75%, at least 80%, at least 85%, at least 90%, at least 95%, at least 98% but less than 100% sequence identity to SEQ ID NO: 32, wherein the protease comprises one or more of the substitutions selected from the group consisting of: S24G/R, S53G, S78N, S101N, G128A/S and Y217Q/L, compared to SEQ ID NO: 32, wherein the positions correspond to the positions of SEQ ID NO: 32.

14. The composition of claim 1, wherein the additional enzyme is a pectinase, and wherein the pectinase has at least 60%, at least 65%, at least 70%, at least 75%, at least 80%, at least 85%, at least 90%, at least 95%, at least 98% or 100% sequence identity to SEQ ID NO: 42.

15. (canceled)

16. A method of cleaning a textile, the method comprising administering a composition of claim 1 to the textile.

17. A method of cleaning a surface, the method comprising administering a composition of claim 1 to the surface.

18. The method of claim 17, wherein the surface is a hard surface.

19. The method of claim 18, wherein the hard surface is selected from floors, tables, walls, roofs, cars and dishes.


SEQUENCE LISTING (42 sequences)

SEQ ID NO: 1 - protein, 222 aa, Bacillus agaradhaerens
HNPVTDEEVYHSFNSHDWQNWNMSDGWKNDDYFFGCHWSQNRVNFYGGQMELSLRTNYSYAPPYNYECAEYTTNNFYGYGLYEVSMKPAKVSGVISSFFTYTGPSYNGAPWDEIDIEFLGNDTTKVQFNYYTDGVGGNEILYDLGFDAADSYNTYAFDWQENYINWYVNGQLVATATENIPSNPSKIMMNIWNTYGIDEWAGRYYGEDANASYNWVRYTPNR

SEQ ID NO: 2 - protein, 351 aa, Bacillus species
YNPVTEDELYHSFDSHDARNWQISDGWRNGDDFFGCHWSQNRVNFNRGEMELSLRTNYSYSAPYNYECAEYATSNFYGYGLYEVSMKPANVSGVISSFFTYTGPSYNGAPWDEIDIEFLGNDTTKVQFNYYTNGVGGNEIIYDLGFDAANSFNTYAFDWQENYISWYVNGNLVATATENIPSNPSKIMMNVWNTYGIDEWAGAYGGEAANATYEWVRYTPNNGNTTPSTAPDFQLQACDYSDSSGITSWSCGVGTFHSSNWIKFDSVDLSTGYNAFAVSYTSPGSGSFDIRLGSPHGQRIGTVNYGATGGWSNYEWSGTPSLDVTVRGAHDIYIVATSGAANLREFWFKNE

SEQ ID NO: 3 - protein, 351 aa, Bacillus species; identical to SEQ ID NO: 2

SEQ ID NO: 4 - protein, 245 aa, Bacillus akibai
AERPIGTTFVETFESYDSERWSKAGVWTNGQMFNATWYPEQVTFSDGKMKLQIDKEDNETASPPYKAGELRTNDFYHYGLFEVSMKPAKSTGTVSSFFTYTGPWDWDNDPWDEIDIEFLGKDTTKIQFNYFTNGVGGNEHYHELGFDAADDFNTYAFEWRPESIRWFVNGELVHTATENIPQTPQKIMMNLWPGIGVDGWTGRFNGEDTPVVTQYDWVKYTPLEELGCYNEKNNKYKKCKKTKVK

SEQ ID NO: 5 - protein, 214 aa, Bacillus mojavensis
QTGGSFFDPFNGYNSGFWQKANGYSNGNMFNCTWRANNVSMTSLGEMRLALTSPSYNKFDCGENRSVQTYGYGLYEVRMKPAKNVGIVSSFFTYTGPTDGTPWDEIDIEFLGKDTTKVQFNYYTNGVGNHEKLVDLGFDAANAYHTYAFDWQPNSIKWYVDGQLKHTATSQIPTTPGKIMMNLWNGTGVDEWLGSYNGVTPLYAHYDWVRYTKK

SEQ ID NO: 6 - protein, 182 aa, Bacillus cibi [sequence omitted]

SEQ ID NO: 7 - protein, 221 aa, Aspergillus oryzae
VPVNPEPDATSVENVALKTGSGDSQSDPIKADLEVKGQSALPFDVDCWAILCKGAPNVLQRVNEKTKNSNRDRSGANKGPFKDPQKWGIKALPPKNPSWSAQDFKSPEEYAFASSLQGGTNAILAPVNLASQNSQGGVLNGFYSANKVAQFDPSKPQQTKGTWFQITKFTGAAGPYCKALGSNDKSVCDKNKNIAGDWGFDPAKWAYQYDEKNNKFNYVGK

SEQ ID NO: 8 - protein, 583 aa, Bacillus species
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVTELKNWGTWYVNTTNIDGFRLDAVKHIKYSFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAKTSNVTFTVNNATTTSGQNVYVVANIPELGNWNTANAIKMNPSSYPTWKATIALPQGKAIEFKFIKKDQAGNVIWESTSNRTYTVPFSSTGSYTASWNVP

SEQ ID NO: 9 - protein, 485 aa, Cytophaga species
AATNGTMMQYFEWYVPNDGQQWNRLRTDAPYLSSVGITAVWTPPAYKGTSQADVGYGPYDLYDLGEFNQKGTVRTKYGTKGELKSAVNTLHSNGIQVYGDVVMNHKAGADYTENVTAVEVNPSNRNQETSGEYNIQAWTGFNFPGRGTTYSNFKWQWFHFDGTDWDQSRSLSRIFKFRGTGKAWDWEVSSENGNYDYLMYADIDYDHPDVVNEMKKWGVWYANEVGLDGYRLDAVKHIKFSFLKDWVDNARAATGKEMFTVGEYWQNDLGALNNYLAKVNYNQSLFDAPLHYNFYAASTGGGYYDMRNILNNTLVASNPTKAVTLVENHDTQPGQSLESTVQPWFKPLAYAFILTRSGGYPSVFYGDMYGTKGTTTREIPALKSKIEPLLKARKDYAYGTQRDYIDNPDVIGWTREGDSTKAKSGLATVITDGPGGSKRMYVGTSNAGEIWYDLTGNRTDKITIGSDGYATFPVNGGSVSVWVQQ

SEQ ID NO: 10 - protein, 485 aa, Bacillus species
HHNGTNGTMMQYFEWYLPNDGNHWNRLRSDASNLKDKGITAVWIPPAWKGASQNDVGYGAYDLYDLGEFNQKGTVRTKYGTRNQLQAAVTALKSNGIQVYGDVVMNHKGGADATEWVRAVEVNPSNRNQEVSGDYTIEAWTKFDFPGRGNTHSNFKWRWYHFDGVDWDQSRQLQNRIYKFRGDGKGWDWEVDTENGNYDYLMYADIDMDHPEVVNELRNWGVWYTNTLGLDGFRIDAVKHIKYSFTRDWLTHVRNTTGKNMFAVAEFWKNDIGAIENYLSKTNWNHSVFDVPLHYNLYNASRSGGNYDMRQIFNGTVVQRHPTHAVTFVDNHDSQPEEALESFVEEWFKPLACALTLTRDQGYPSVFYGDYYGIPTHGVPAMKSKIDPILEARQKYAYGKQNDYLDHHNMIGWTREGNTAHPNSGLATIMSDGPGGNKWMYVGRNKAGQVWRDITGNRSGTVTINADGWGNFSVNGGSVSIWVNN

SEQ ID NO: 11 - protein, 485 aa, Bacillus species
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 12 - protein, 485 aa, Bacillus subtilis
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK

SEQ ID NO: 13 - protein, 485 aa, Bacillus species
HHDGTNGTIMQYFEWNVPNDGQHWNRLHNNAQNLKNAGITAIWIPPAWKGTSQNDVGYGAYDLYDLGEFNQKGTVRTKYGTKAELERAIRSLKANGIQVYGDVVMNHKGGADFTERVQAVEVNPQNRNQEVSGTYQIEAWTGFNFPGRGNQHSSFKWRWYHFDGTDWDQSRQLANRIYKFRGDGKAWDWEVDTENGNYDYLMYADVDMDHPEVINELNRWGVWYANTLNLDGFRLDAVKHIKFSFMRDWLGHVRGQTGKNLFAVAEYWKNDLGALENYLSKTNWTMSAFDVPLHYNLYQASNSSGNYDMRNLLNGTLVQRHPSHAVTFVDNHDTQPGEALESFVQGWFKPLAYATILTREQGYPQVFYGDYYGIPSDGVPSYRQQIDPLLKARQQYAYGRQHDYFDHWDVIGWTREGNASHPNSGLATIMSDGPGGSKWMYVGRQKAGEVWHDMTGNRSGTVTINQDGWGHFFVNGGSVSVWVKR

SEQ ID NO: 14 - protein, 485 aa, Bacillus species
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGHGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGPQHDYIDHPDVIGWTREGDSSAAKSGLAALITDGPGGSKRMYAGLKNAGETWYDITGNRSDTVKIGSDGWGEFHVNDGSVSIYVQK

SEQ ID NO: 15 - protein, 485 aa, Artificial Sequence
  Synthetic Construct
HHDGTNGTIMQYFEWNVPNDGQHWNRLHNNAQNLKNAGITAIWIPPAWKGTSQNDVGYGAYDLYDLGEFNQKGTVRTKYGTKAELERAIRSLKANGIQVYGDVVMNHKGGADFTERVQAVEVNPQNRNQEVSGTYQIEAWTGFNFPGRGNQHSSFKWRWYHFDGTDWDQSRQLANRIYKFRGDGKAWDWEVDTENGNYDYLMYADVDMDHPEVINELNRWGVWYANTLNLDGFRLDAVKHIKFSFMRDWLGHVRGQTGKNLFAVAEYWKNDLGALENYLSKTNWTMSAFDVPLHYNLYQASNSSGNYDMRNLLNGTLVQRHPSHAVTFVDNHDTQPGEALESFVQGWFKPLAYATILTREQGYPQVFYGDYYGIPSDGVPSYRQQIDPLLKARQQYAYGTQHDYLDNQDVIGWTREGDSAHAGSGLATVMSDGPGGSKTMYVGTAHAGQVFKDITGNRTDTVTINSAGNGTFPCNGGSVSIWVKQ

SEQ ID NO: 16 - protein, 773 aa, Bacillus species
AEGNTREDNFKHLLGNDNVKRPSEAGALQLQEVDGQMTLVDQHGEKIQLRGMSTHGLQWFPEILNDNAYKALANDWESNMIRLAMYVGENGYASNPELIKSRVIKGIDLAIENDMYVIVDWHVHAPGDPRDPVYAGAEDFFRDIAALYPNNPHIIYELANEPSSNNNGGAGIPNNEEGWNAVKEYADPIVEMLRDSGNADDNIIIVGSPNWSQRPDLAADNPINDHHTMYTVHFYTGSHAASTESYPPETPNSERGNVMSNTRYALENGVAVFATEWGTSQANGDGGPYFDEADVWIEFLNENNISWANWSLTNKNEVSGAFTPFELGKSNATNLDPGPDHVWAPEELSLSGEYVRARIKGVNYEPIDRTKYTKVLWDFNDGTKQGFGVNSDSPNKELIAVDNENNTLKVSGLDVSNDVSDGNFWANARLSADGWGKSVDILGAEKLTMDVIVDEPTTVAIAAIPQSSKSGWANPERAVRVNAEDFVQQTDGKYKAGLTITGEDAPNLKNIAFHEEDNNMNNIILFVGTDAADVIYLDNIKVIGTEVEIPVVHDPKGEAVLPSVFEDGTRQGWDWAGESGVKTALTIEEANGSNALSWEFGYPEVKPSDNWATAPRLDFWKSDLVRGENDYVAFDFYLDPVRATEGAMNINLVFQPPTNGYWVQAPKTYTINFDELEEANQVNGLYHYEVKINVRDITNIQDDTLLRNMMIIFADVESDFAGRVFVDNVRFEGAATTEPVEPEPVDPGEETPPVDEKEAKKEQKEAEKEEKEE

SEQ ID NO: 17 - protein, 435 aa, Humicola insolens
MARGTALLGLTALLLGLVNGQKPGETKEVHPQLTTFRCTKRGGCKPATNFIVLDSLSHPIHRAEGLGPGGCGDWGNPPPKDVCPDVESCAKNCIMEGIPDYSQYGVTTNGTSLRLQHILPDGRVPSPRVYLLDKTKRRYEMLHLTGFEFTFDVDATKLPCGMNSALYLSEMHPTGAKSKYNPGGAYYGTGYCDAQCFVTPFINGLGNIEGKGSCCNEMDIWEANSRASHVAPHTCNKKGLYLCEGEECAFEGVCDKNGCGWNNYRVNVTDYYGRGEEFKVNTLKPFTVVTQFLANRRGKLEKIHRFYVQDGKVIESFYTNKEGVPYTNMIDDEFCEATGSRKYMELGATQGMGEALTRGMVLAMSIWWDQGGNMEWLDHGEAGPCAKGEGAPSNIVQVEPFPEVTYTNLRWGEIGSTYQEVQKPKPKPGHGPRSD

SEQ ID NO: 18 - protein, 305 aa, Humicola insolens
MRSSPLLRSAVVAALPVLALAADGRSTRYWDCCKPSCGWAKKAPVNQPVFSCNANFQRITDFDAKSGCEPGGVAYSCADQTPWAVNDDFALGFAATSIAGSNEAGWCCACYELTFTSGPVAGKKMVVQSTSTGGDLGSNHFDLNIPGGGVGIFDGCTPQFGGLPGQRYGGISSRNECDRFPDALKPGCYWRFDWFKNADNPSFSFRQVQCPAELVARTGCRRNDDGNFPAVQIPSSSTSSPVNQPTSTSTTSTSTTSSPPVQPTTPSGCTAERWAQCGGNGWSGCTTCVAGSTCTKINDWYHQCL

SEQ ID NO: 19 - protein, 299 aa, Thielavia terrestris
MRSTPVLRTTLAAALPLVASAASGSGQSTRYWDCCKPSCAWPGKAAVSQPVYACDANFQRLSDFNVQSGCNGGSAYSCADQTPWAVNDNLAYGFAATSIAGGSESSWCCACYALTFTSGPVAGKTMVVQSTSTGGDLGSNHFDIAMPGGGVGIFNGCSSQFGGLPGAQYGGISSRDQCDSFPAPLKPGCQWRFDWFQNADNPTFTFQQVQCPAEIVARSGCKRNDDSSFPVFTPPSGGNGGTGTPTSTAPGSGQTSPGGGSGCTSQKWAQCGGIGFSGCTTCVSGTTCQKLNDYYSQCL

SEQ ID NO: 20 - protein, 269 aa, Thermomyces lanuginosus [sequence omitted]

SEQ ID NO: 21 - protein, 298 aa, Bacillus bogoriensis
ANSGFYVSGTTLYDANGNPFVMRGINHGHAWYKDQATTAIEGIANTGANTVRIVLSDGGQWTKDDIHTVRNLISLAEDNHLVAVLEVHDATGYDSIASLNRAVDYWIEMRSALIGKEDTVIINIANEWFGSWEGDAWADGYKQAIPRLRNAGLNHTLMVDAAGWGQFPQSIHDYGREVFNADPQRNTMFSIHMYEYAGGNASQVRTNIDRVLNQDLALVIGEFGHRHTNGDVDEATIMSYSEQRGVGWLAWSWKGNGPEWEYLDLSNDWAGNNLTAWGNTIVNGPYGLRETSRLSTVF

SEQ ID NO: 22 - protein, 330 aa, Paenibacillus woosongensis
IRTGTLNNPEATAEARALMNYLLSQYGQKIISGQQTLEDVEWIKQQTGKYPAIFSTDLMDYSPSRVDHGASSTEVEKMIEWYKRGGIVSLCWHWNAPKGIGGNEPGNEWWRGFYTEFTTFDVEYALNHPDSEDYQLLIRDIDAIAVQLKRLQEANVPVLWRPLHEAEGTWFWWGAKGPEPAKQLYRLMYDRLTNDHKLNNLIWVWNSEKKDWYPGDDVVDMVSVDIYNPAGDYNPSIAKYEALVSLADNKKMAALAENGPIPDPDALQEYGADWSFFSTWTGDYIRDGKTNTIEHLKKVYQHDYVITLDELPADCTPILMIRQRMVNQQG

SEQ ID NO: 23 - protein, 312 aa, Paenibacillus illinoisensis
ASPQAKALMKFMTNQYGKKIISGQQTLEDAAWIYQQTGKYPALVSSDLMDYSPSRVENGSTSNEVEKMMEWYKRGGIVSLSWHWNAPKGIGSNEPGHEWWRGFNTEFTTFDVEYALNHPESEDYKLLIRDIDAIATQLKRLQEHHIPVLWRPLHEAEGGWFWWGAKGPEPAKKLYRLMYERLTEKHGLNNLIWVWNSVKEEWYPGDDVVDMVSVDIYNPPGDYSPNIAKYDELLFLSKHKKLVALAENGPIPDPDLLQTYGAHWSYFNTWTGDVLRDGKTNTKEHLKKVYNHDNVITLDELPKGLYDSPRWK

SEQ ID NO: 24 - protein, 699 aa, Neobulgaria species
TVFEAEKGTLAGGLTIATDVTGYTGTGYVTNFADAAALLTFTVNGLTAGSYDLTLTYSAQYGDKFTTVSVNGASGIEVAITNVTTATWTTATIGTFTLTAGDNTVSCADDWGWYLIDSLTVIPTPAKPITIVDVSNGATAQAEDGILTGTTVGTTTAGYTGTGYVTGFTATGTQVTINLSSTKQALYDVVVRYAAIYGQKYTTMQLNGVGGSEILLLDTTTATSPWANATAGQVLLASGNNTLTFMNDWGWYFIDAVYVTPSPAPAPHKVTNALVDAKALASTHALFNTLLAKYGSGDIFSGQADPTGVTWIESNLGTTKTPAIIGLDMIEYSPTRVLYGSTSTAVEDAIAFDKRGGMVAFQWHWNAPADLINNDTVPWWKGFYSYGTTFNLTAALANPSGSDYALLISDMDAIAVQLLRLQAAGVPVLWRPLHEADGTWFWWGNFGAASCVSLYRIMYDRYTNYHGLHNLIWVWNSVTPSWYPGADVVDILGYDSYPAVGDHGPVSSQYNALITLGGDTKLVTLPEVGNIPDPAILKLYHADWSYFVTWNQDYILTDTYNPLAFKQQVYNDPTVLKLTDLGNWKGAATSTIVSSTSKVSTTTSSLITSTTKKTSSSTVVSTTSSTVKTTSTTSKVSSSTTKVSSTTKVTTTSTTTSAVATATAGHWGQCGGTGWTGPTVCASGFTCTAVSPPYYYQCL

SEQ ID NO: 25 - protein, 452 aa, Preussia aemulans
QTVIYQAEQAKLSGVTVEFSIIKQVVGTGYVEGFDESTDSITFTVESTTAALYDLALTYNGPYGDKYTNVVLNNAAGSQVSLPATTAWTTVPAGQVLLNAGANTIQIQNNWGWYLVDSISLKPAATRGAHQITTKPVNKNANSDAKALLKYLGSIYGKKILSGQQDLSSLDWVTKNVGKTPAVLGLDTMDYSESRKSRGAVSTDVDKAIAFAKKGGIVTFCWHWGAPTGLFDSAAQPWYRGFYTDATDFNIETALKDTTNANYTLLMKDIDTIAVQLKKLQDAGVPVIWRPLHEAEGGWFWWGAKGPEPAKKLWKIMYDRLTNQHGLNNLVWTWNSVAPNWYPGDDTVDIVSADTYSQGDHGPISATYNNLLALTNDTKIIAAAEIGSVMEPAQLQAYQADWVYFCVWSGEFIDGGVWNSLDFLKKVYNDPYVLTLDEIQGWKTARGKPRVS

SEQ ID NO: 26 - protein, 312 aa, Yunnania penicillate
APSTTPVNEKATDAAKNLLSYLVEQAANGVTLSGQQDLESAQWVSDNVGKWPAILGIDFMDYSPSRVEYGAVGSTVPDAISYDSDGGIVTFCWHWGSPSGTYNTTDQPWWSNFYTEATAFDIAAAMDDPDSADYNLLVRDIDAISELLLQLQDLDIPILWRPLHEAEGGWFWWGAKGPEACIALYRLMFDRMTNHHGLNNLLWVWNSVDPSWYPGNDVVDIVSADIYADAGDHSPQEETFASLQSLTGDTKLVALGEVGNIPDPASTGGVADWAYWVTWNGDFIKGEDYNPLEYKKEVFSAENIITRDEVDV

SEQ ID NO: 27 - protein, 327 aa, Myrothecium roridum
GTIENRQWLTYNPVDSAATTEARALLRYIQSQYGWRYLSGQQERAEVQWLKSNIGKTPAIQGSDLIDYSPSRVSYGATSTAVEDAIAFDRQGGIVTFTWHWNAPNCLYNSADQPWYFGFYTKATCFNIQAALAQGSNGADYKLLIRDIDAIAVQLKRLRDAKVPILFRPLHEPDGAWFWWGAKGSGPFKQLWDILYDRLTKYHGLHNMLWVCNTEKSDWYPGNNKCDIATTDVYVNAGDHSVQKSHWDALYGVSGGQRILALGEVGVIPDPERQASENVPWAYWMTWNGYFIRDGNYNSRNFLQSTFSNARVVTLDGTSPLGNWKSS

SEQ ID NO: 28 - protein, 452 aa, Chaetomium brasiliense
VPCGGGSNSGPRTYEAEDADLTGTNIDTAQSGFTGSGYVTGFDQATDKVTFKVDSPSLKLYDLSIRVAAIYGEKRTNVVLNNGASSEVYFAASETFTTVAAGQVLLNEGANTIDLVSNWGWYLIDSITLTPSTQRPPHNINPSPVNPSANADAKGLYTYLRSIYGKKILSGQQELSWSNWITTQTGKTPALVSVDLMDYSPSRVERGTVGTAVEEAITHAQRGGIVSVLWHWNAPTGLYDTEENKWWSGFYTRATDFDVAAALSSTTNANYTLILRDIDAIAVQLKKLQDAGVPVLFRPLHEAEGGWFWWGAKGAEPCKKLYALLYDRLTNYHKINNLIWVWNSILEEWYPGDATVDILSADVYAQGNGPISTQYNQLIELGKDKKMIAAAEVGAAPLPNLLQAYEAHWLWFTVWGDTFINNAEWNSVDVLKQVYTSDYVLTLDEIQGWRGA

SEQ ID NO: 29 - protein, 481 aa, Ascobolus stictoideus
PYGSKYTRISYNGATGGDISLPETTEWATVNAGQALLNAGSNTIKLHNNWGWYLIDAVILTPSVPRPPHQVTDALVNTNSNAVTKQLMKFLVSKYHKAYITGQQELHAHQWVEKNVGKSPAILGLDFMDYSPSRVEFGTTSQAVEQAIDFDKRGGIVTFAWHWNAPSGLINTPGSEWWRGFYTEHTTFDVAAALQNTTNANYNLLIRDIDAIAVQLKRLQTAGVPVLWRPLHEAEGGWFWWGAKGPEPAKKLYKILYDRLTNYHKLNNLIWVWNSVAKDWYPGDEIVDVLSFDSYPAQPGDHGPVSAQYNALVELGKDKKLIAATEVGTIPDPDLMQLYESYWSFFVTWEGEFIENGVHNSLEFLKKLYNNSFVLNLDTIQGWKNGAGSSTTTVKSTTTTPTTTIKSTTTTPVTTPTTVKTTTTPTTTATTVKSTTTTAGPTPTAVAGRWQQCGGIGFTGPTTCEAGTTCNVLNPYYSQCL

SEQ ID NO: 30 - protein, 526 aa, Chaetomium virescens
PRDPGATARTFEAEDATLAGTNVDTALSGFTGTGYVTGFDQAADKVTFTVDSASTELYDLSIRVAAIYGDKRTSVVLNGGASSEVYFPAGETWTNVAAGQLLLNQGSNTIDIVSNWGWYLIDSITLTPSTPRPAHQINEAPVNAAADKNAKALYSYLRSIYGKKILSGQQELSLSNWIAQQTGKTPALVSVDLMDYSPSRVERGTVGTAVEEAIQHHNRGGIVSVLWHWNAPTGLYDTEEHRWWSGFYTSATDFDVAAALSSTTNANYTLLIRDIDAIAVQLKRLQSAGVPVLFRPLHEAEGGWFWWGAKGPEPAKKLWGILYDRVTNHHQINNLLWVWNSILPEWYPGDATVDILSADVYAQGNGPMSTQYNQLIELGKDKKMIAAAEVGAAPLPDLLQAYEAHWLWFTVWGDSFINNADWNSLDTLKKVYTSDYVLTLDEIQGWQGSTPSATTTSSTTTPSATTTTTTPSTTATTATPSATTTASPVTYAEHWGQCAGKGWTGPTTCRPPYTCKYQNDWYSQCL

SEQ ID NO: 31 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 32 - protein, 275 aa, Bacillus amyloliquefaciens [sequence omitted]

SEQ ID NO: 33 - protein, 311 aa, Bacillus species [sequence omitted]

SEQ ID NO: 34 - protein, 269 aa, Bacillus gibsonii
QQTVPWGITRVQAPAVHNRGVTGSGVRVAILDSGISTHSDLTIRGGASFVPGEPTTADLNGHGTHVAGTVAALNNSIGVIGVAPSADLYAVKVLGANGRGSVSGIAQGLEWAAANNMHIANMSLGSDAPSITLERAVNYATSQGVLVIAATGNNGSGSVGYPARYANAMAVGATDQNNRRANFSQYGTGIDIVAPGVNVQSTYPGNRYASLNGTSMATPHVAGAAALVKQRYPSWNATQIRNHLKNTATNLGNSSQFGSGLVNAEAATR

SEQ ID NO: 35 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGRGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 36 - protein, 274 aa, Bacillus licheniformis
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 37 - protein, 524 aa, Paenibacillus polymyxa
VVHGQTAKTITIKVDTFKDRKPISPYIYGTNQDLAGDENMAARRLGGNRMTGYNWENNMSNAGSDWQHSSDNYLCSNGGLTQAECEKPGAVVTSFHDQSLKLGTYSLVTLPMAGYVAADGNGSVQESEAAPSARWNQVVNAKNAPFQLQPDLNDNYVYVDEFVHFLVNKYGTASTKAGVKGYALDNEPALWSHTHPRIHPEKVGAKELVDRSVSLSKAVKAIDAGAEVFGPVLYGFGAYKDLQTAPDWDSVKGNYSWFVDYYLDQMRLSSQVEGKRLLDVFDVHWYPEAMGGGIRITNEVGNDETKKARMQAPRTLWDPTYKEDSWIAQWFSEFLPILPRLKQSVDKYYPGTKLAMTEYSYGGENDISGGIAMTDVLGILGKNDVYMANYWKLKDGVNNYVSAAYKLYRNYDGKNSTFGDTSVSAQTSDIVNSSVHASVTNASDKELHLVVMNKSMDSAFDAQFDLSGAKTYISGKVWGFDKNSSQIKEAAPITQISGNRFTYTVPPLTAYHIVLTTGNDTSPV

SEQ ID NO: 38 - protein, 214 aa, Melanocarpus albomyces
ANGQSTRYWDCCKPSCGWRGKGPVNQPVYSCDANFQRIHDFDAVSGCEGGPAFSCADHSPWAINDNLSYGFAATALSGQTEESWCCACYALTFTSGPVAGKTMVVQSTSTGGDLGSNHFDLNIPGGGVGLFDGCTPQFGGLPGARYGGISSRQECDSFPEPLKPGCQWRFDWFQNADNPSFTFERVQCPEELVARTGCRRHDDGGFAVFKAPSA

SEQ ID NO: 39 - protein, 297 aa, Paenibacillus species
MATGFYVSGNKLYDSTGKPFVMRGVNHGHSWFKNDLNTAIPAIAKTGANTVRIVLSNGSLYTKDDLNAVKNIINVVNQNKMIAVLEVHDATGKDDYNSLDAAVNYWISIKEALIGKEDRVIVNIANEWYGTWNGSAWADGYKKAIPKLRNAGIKNTLIVDAAGWGQFPQSIVDYGQSVFAADSQKNTVFSIHMYEYAGKDAATVKANMENVLNKGLALIIGEFGGYHTNGDVDEYAIMRYGQEKGVGWLAWSWYGNSSGLNYLDMATGPNGSLTSFGNTVVNDTYGIKNTSQKAGIF

SEQ ID NO: 40 - protein, 464 aa, Bacillus hemicellulosilyticus
QTHSGFYIEGSTLYDANGEPFVMRGINHGHAWYKHDSNVAIPAIANQGANTIRIVLSDGGQWAKDDINTLNQVLDLAEEHEMIAVVEVHDATGSNSMADLNRAVDYWIEMKDALIGKEDRVIINIANEWYGAWDGQGWANGYKEVIPRLRNAGFTHTLMVDAAGWGQYPQSIHDYGQEVFNADPLANTMFSIHMYEYAGGNASMVQSNIDGVVDQGLALVIGEFGHMHTDGDVDEATILSYSQQRGVGWLAWSWKGNGTQWEYLDLSYDWQGTNLTSWGNTIVHGPNGLLETSIPSSIFHTAPNNGDPPPHNGNETILYDFEHGTQGWSGSSLLGGPWTTNEWSTNGNHSLKADIFLSANSKHELAKVENRNLSGYSTLQATVRHAHWGNVGNLTARMYVKTGSNYSWFNGDPIPVNSANGTTVTLPLSSIPNLNDVKEIGVEFIGASNSNGQTAIYLDHVTIQ

SEQ ID NO: 41 - protein, 484 aa, Bacillus sp
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVTELKNWGTWYVNTTNIDGFRLDAVKHIKYSFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 42 - protein, 399 aa, Bacillus subtilis [sequence omitted]